(19) United States
(12) Patent Application Publication
(10) Pub. No.: US 2025/0236854 A1
(43) Pub. Date: Jul. 24, 2025

(54) CRISPR/CAS9-BASED FUSION PROTEINS FOR MODULATING GENE EXPRESSION AND METHODS OF USE

(71) Applicant: Glaxosmithkline Intellectual Property Development Limited GSK Medicines Research Centre, Stevenage (GB)

(72) Inventors: Syandan Chakraborty, Medford, MA (US); Rajarshi Choudhury, Medford, MA (US); William Walker, Collegeville, PA (US)

(21) Appl. No.: 18/698,042

(22) PCT Filed: Oct. 3, 2022
(86) PCT No.: PCT/IB2022/059433

§ 371 Date: Apr. 3, 2024

Related U.S. Application Data
(60) Provisional application No. 63/252,376, filed on Oct. 5, 2021.

(51) Int. Cl. C12N 9/22; C07K 14/47; C12N 15/11; C12N 15/86; C12N 15/90

(52) U.S. Cl. CPC C12N 9/22; C07K 14/4703; C12N 15/11; C12N 15/86; C12N 15/907; C07K 2319/09; C07K 2319/60; C12N 2310/20; C12N 2740/15043

(57) ABSTRACT

The present disclosure generally relates to methods and compositions used for modulating or controlling gene expression involving sequence targeting, genome perturbation or gene-editing, that relate to Clustered Regularly Interspaced Short Palindromic Repeats (CRISPR) and components thereof. In some embodiments, compositions comprising a catalytically inactive Cas9 (dCas9) fusion protein and methods for modulating expression of a gene of interest are disclosed.

### CROSS REFERENCE TO RELATED APPLICATIONS

This application claims the benefit of U.S. Provisional Application No. 63/252,376, filed 5 Oct. 2021, the disclosure of which is incorporated herein in it's entirety.

### SEQUENCE LISTING

The instant application contains a Sequence Listing, which has been submitted electronically in computer readable form in XML file format and is hereby incorporated by reference in its entirety. Said XML file, created on 14 Sep. 2022, is named “70052WO01_SL.xml” and is 124,165 bytes in size.

### FIELD OF THE INVENTION

The present disclosure generally relates to methods and compositions used for modulating or controlling gene expression involving sequence targeting, genome perturbation or gene-editing, that relate to Clustered Regularly Interspaced Short Palindromic Repeats (CRISPR) and components thereof. In particular, the present disclosure relates to compositions comprising a catalytically inactive Cas9 (dCas9) fusion protein and methods for modulating expression of a gene of interest.

### BACKGROUND TO THE INVENTION

An RNA-guided CRISPR-Cas9 system has emerged as a promising platform for programmable targeted gene regulation. Fusion of catalytically inactive Cas9 (dCas9) to the Krüppel-associated box (KRAB) domain generates a synthetic repressor (i.e., dCas9-KRAB fusion protein) capable of silencing target genes, which has been deemed as the current gold standard for dCas9-based repression studies. This use of dCas9 to repress gene expression was termed CRISPR interference (CRISPRi) (Qi et al. Cell. 2013). Although it has been widely adopted, the dCas9-KRAB system suffers from inefficient knockdown and poor performance compared with that of Cas9 nuclease-based methods. Precise genome targeting technologies are needed to enable systematic determination of causal genetic variations. Thus, there remains a need for alternative or improved compositions and methods for the programmable and quantitiative control of endogenous gene expression.

### SUMMARY OF THE INVENTION

Aspects of the present disclosure relate to fusion proteins comprising a dCas9 protein and two or more repressor domains as well as methods of silencing endogenous genes of a subject. In an additional aspect of the present disclosure, the dCas9 protein comprises one or more mutations and may be used as a generic DNA binding protein with fusion to a functional domain. The mutations may include, but are not limited to, mutations in one of the catalytic domains (e.g., D10 and H840 in the RuvC and HNH catalytic domains, respectively). Further mutations have been characterized and may be used in one or more compositions of the disclosure. In one aspect of the disclosure, the mutated Cas9 or catalytically inactive Cas9 (i.e., dCas9) protein may be fused to a repressor or regulatory domains of other proteins, e.g., such as a transcriptional repression domain. In one aspect, the transcriptional repression domain include, but is not limited to, ZIM3 Krüppel-associated box (ZIM3 KRAB domain), a transcription repression domain of methyl-CpG binding protein 2 (MeCP2 domain), and/or an interaction domain of Friend of GATA1 (FOG1 domain). The interaction domain of FOG1 comprises a repression domain of FOG1, an N-terminal portion of FOG1, and/or the N-terminal 45 residues of FOG1 (e.g., residues 1-45 of FOG1). Other aspects of the disclosure relate to the dCas9 protein being fused to domains which include but are not limited to a transcriptional activator, repressor, a recombinase, a transposase, a histone remodeler, a demethylase, a DNA methyltransferase, a cryptochrome, a light inducible/controllable domain or a chemically inducible/controllable domain.

In certain embodiments, the dCas9 comprises one or more mutations selected from the group consisting of D10A, E762A, H840A, N854A, N863A or D986A and/or the one or more mutations is in a RuvC1 or HNH domain of the Cas9 protein or is a mutation as otherwise as discussed herein (e.g., mutations can be made with reference to SEQ ID NO: 54). Cas9 sequences and structures from different species are known in the art (see e.g., Jinek et al. Science. 2012; see also SEQ ID NOs: 54-57). In some embodiments, the Cas9 has one or more mutations in a catalytic domain, wherein when transcribed, the tracr mate sequence hybridizes to the tracr sequence and the guide sequence directs sequence-specific binding of a CRISPR complex to the target sequence, and wherein the fusion protein comprises two or more functional domains. In some embodiments, the two or more functional domains include a transcriptional repression domain, preferably ZIM3 Krüppel-associated box (ZIM3 KRAB domain), and/or a transcription repression domain of methyl-CpG binding protein 2 (MeCP2 domain), and/or an interaction domain of Friend of GATA1 (FOG1 domain). In some embodiments, the fusion protein comprises dCas9 fused to ZIM3 Krüppel-associated box (ZIM3 KRAB domain), a transcription repression domain of methyl-CpG binding protein 2 (MeCP2 domain), and an interaction domain of Friend of GATA1 (FOG1 domain). In some embodiments, the fusion protein comprises a fluorescent marker (FM). In one embodiment, the FM comprises at least one of a monomeric blue fluorescent protein (mTagBFP), blue fluorescent protein (BFP), red fluorescent protein (RFP), yellow fluorescent protein (YFP), green fluorescent protein (GFP), and enhanced green fluorescent protein (eGFP). The FM may be used to improve repression by increasing spacing between the domains or increasing flexibility of the other attached functional domains. Also, the FM may help track dCas9 expression and nuclear localization after transduction.

In one aspect, the fusion protein includes a dCas protein and two or more functional domains, or a nucleic acid encoding the fusion protein comprising a dCas protein and two or more functional domains. In one embodiment, the dCas protein and the two or more functional domains are linked covalently. In one embodiment, the two or more functional domains are covalently fused to the dCas protein directly. In one embodiment, the two or more functional domains are covalently fused to the dCas protein indirectly, e.g., via a linker, a peptide, a nuclear localization sequence (NLS), or via a second functional domain. In one embodiment, the two or more functional domains are at the N-terminus and/or C-terminus of the dCas protein. In one embodiment, the dCas protein and the two or more functional domains are linked in tandem. In one embodiment, a nucleic acid encoding a dCas protein is operably linked to two or more functional domains. In one embodiment, the dCas protein and the two or more functional domains are fused to at least one fluorescent marker (FM). In one embodiment, the at least one FM may bring the dCas protein and the two or more functional domains into close proximity.

In one aspect, the disclosure relates to the use of fusion proteins comprising a dCas9 protein and two or more repressor domains (or nucleic acid encoding the fusion proteins) in a method of repressing expression of a gene in a subject. In one embodiment, a composition comprising the fusion protein according to the present disclosure or the polynucleotide encoding the fusion protein, and one or more gRNAs that bind the dCas9 protein are administered to a subject. In one embodiment, the one or more gRNAs comprises a sequence that has sufficient complementarity with a target polynucleotide sequence. In one embodiment, the one or more gRNAs are capable of hybridizing with the target sequence. In one embodiment, the composition is packaged in a viral vector. In one embodiment, the viral vector is a lentiviral vector.

In one aspect, a viral vector comprising the polynucleotide according to the present disclosure; optionally further comprising one or more gRNAs that bind to the dCas9 protein. In one embodiment, the viral vector is a lentiviral vector. In one aspect, a pharmaceutical composion comprising the viral vector comprising the polynucleotide encoding the fusion protein according to the present disclosure; optionally further comprisingone or more gRNAs that bind to the dCas9 protein is also provided herein. In one embodiment, the viral vector is a lentiviral vector.

### DESCRIPTION OF DRAWINGS/FIGURES

FIG. 1A shows nucleic acid constructs used to generate a triple inhibitory domain dCas9 fusion construct.

FIG. 1B shows final triple inhibitory domain dCas9 fusion construct comprising triple repressor domains and a mTagBFP (also referred to as “Triple Repressor” or “Triple Rep”).

FIG. 1C shows a putative structure of a dCas9 fusion protein comprising triple repressor domains and a mTagBFP.

FIG. 1D shows a lentiviral vector having the triple repressor with mTagBFP sequences. The total payload size is about 14853 bp.

FIG. 2A shows a schematic drawing of CRISPRi reporter assay system. CRISPRi activity is indicated by a decrease in relative GFP (normalized to RFP) expression measured by flow cytometry

FIG. 2B shows the CRISPRi activity of the varous KRAB-based dCas9 (e.g., UCOE-KRAB, KOX1-KRAB, and ZIM3 KRAB) and the CRISPRi activity of Triple Repressor dCas9 (e.g., Clone 9 and Clone 11) in an adherent cell line (A549) using a reporter assay.

FIG. 2C shows the CRISPRi activity of the varous KRAB-based dCas9 (e.g., UCOE-KRAB, KOX1-KRAB, and ZIM3 KRAB) and the CRISPRi activity of Triple Repressor dCas9 (e.g., Clone 9 and Clone 11) in an adherent cell line (HEK293T) using a reporter assay.

FIG. 2D shows the CRISPRi activity of the varous KRAB-based dCas9 (e.g., UCOE-KRAB, KOX1-KRAB, and ZIM3 KRAB) and the CRISPRi activity of Triple Repressor dCas9 (e.g., Clone 9 and Clone 11) in a suspension cell line (K562) using a reporter assay.

FIG. 3A shows the CRISPRi activity of ZIM3 KRAB dCas9 and the CRISPRi activity of Triple Repressor dCas9 by measuring percent residual RAB1A expression in an adherent cell line (HEK293T).

FIG. 3B shows the CRISPRi activity of ZIM3 KRAB dCas9 and the CRISPRi activity of Triple Repressor dCas9 by measuring percent residual RAB1A expression in an adherent cell line (A549).

FIG. 3C shows the CRISPRi activity of ZIM3 KRAB dCas9 and the CRISPRi activity of Triple Repressor dCas9 by measuring percent residual RAB1A expression in an suspension cell line (K562).

FIG. 4A shows the CRISPRi activity of ZIM3 KRAB dCas9 and the CRISPRi activity of Triple Repressor dCas9 by measuring percent residual ATF4 expression in an adherent cell line (A549) using two gRNAs.

FIG. 4B shows the CRISPRi activity of ZIM3 KRAB dCas9 and the CRISPRi activity of Triple Repressor dCas9 by measuring percent residual EZH2 expression in an adherent cell line (A549) using two gRNAs.

FIG. 4C shows the CRISPRi activity of ZIM3 KRAB dCas9 and the CRISPRi activity of Triple Repressor dCas9 by measuring percent residual HDAC1 expression in an adherent cell line (A549) using two gRNAs.

FIG. 4D the CRISPRi activity of ZIM3 KRAB dCas9 and the CRISPRi activity of Triple Repressor dCas9 by measuring percent residual LMNA expression in an adherent cell line (A549) using two gRNAs.

FIG. 5A shows the CRISPRi activity of ZIM3 KRAB dCas9 and the CRISPRi activity of Triple Repressor dCas9 by measuring percent residual ATF4 expression in an adherent cell line (HEK293T) using two gRNAs.

FIG. 5B shows the CRISPRi activity of ZIM3 KRAB dCas9 and the CRISPRi activity of Triple Repressor dCas9 by measuring percent residual EZH2 expression in an adherent cell line (HEK293T) using two gRNAs.

FIG. 5C shows the CRISPRi activity of ZIM3 KRAB dCas9 and the CRISPRi activity of Triple Repressor dCas9 by measuring percent residual HDAC1 expression in an adherent cell line (HEK293T) using two gRNAs.

FIG. 5D the CRISPRi activity of ZIM3 KRAB dCas9 and the CRISPRi activity of Triple Repressor dCas9 by measuring percent residual LMNA expression in an adherent cell line (HEK293T) using two gRNAs.

### DETAILED DESCRIPTION OF THE INVENTION

CRISPRs described herein refer to loci containing multiple short direct repeats that are found in the genomes of bacteria and archaea. The CRISPR system is a microbial “defense” system that fights against invading phages and plasmids (e.g., a form of an adaptive immune system). The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA (i.e., spacers) are incorporated into the genome between CRISPR repeats, and serve as a ‘memory’ of past exposures. Cas9 protein forms a complex with the 3′ end of the guide RNA (gRNA), and the protein-RNA complex recognizes its genomic target by complementary base pairing between the 5′ end of the gRNA sequence and a predefined 20 bp DNA sequence (i.e., a protospacer). This complex is directed to homologous loci of pathogen DNA via regions encoded within the CRISPR RNA (crRNA) (i.e., the protospacers) and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed gRNA, the Cas9 nuclease can be directed to new genomic targets. Cas9 protein may be mutated through genetic engineering such that Cas9 becomes catalytically inactive. A Cas9 protein from S. pyogenes having catalytically inactive endonuclease domain has been used to silence gene expression through steric hindrance.

Aspects of the present disclosure relate to fusion proteins comprising a dCas9 protein linked directly or indirectly to to two or more repressor domains and nucleic acid molecules coding therefor, as well as methods of silencing endogenous genes of a subject.

In one embodiment, a fusion protein for repressing expression of a gene is provided. The fusion protein comprises a catalytically inactive Cas9 (dCas9) protein and two or more repressor domains, wherein the two or more repressor domains are selected from the group consisting of: a Krüppel-associated box domain of ZIM3 gene (ZIM3 KRAB domain); a transcription repression domain of methyl-CpG binding protein 2(MeCP2 domain); and an interaction domain of Friend of GATA1 (FOG1 domain). In one embodiment, the dCas9 protein is linked directly or indirectly to ZIM3 KRAB domain and MeCP2 domain, ZIM3 KRAB domain and FOG1 domain, or MeCP2 domain and FOG1 domain. In one embodiment, the dCas9 protein is linked directly or indirectly to ZIM3 KRAB domain, MeCP2 domain, and FOG1 domain. In one embodiment, the ZIM3 KRAB domain is linked adjacent to the N-terminus of the dCas9 protein, and/or wherein MeCP2 domain is linked adjacent to the N-terminus of dCas9, and/or wherein FOG1 domain is linked adjacent to the C-terminus of the dCas9 protein. Yet another embodiment, the dCas9 protein comprises at least one domain selected from the group consisting of: a Rec1 domain, a bridge helix domain, and a protospacer adjacent motif interacting domain.

In an additional aspect of the present disclosure, a dCas9 protein may comprise one or more mutations and may be used as a generic DNA binding protein with fusion to a functional domain. The mutations may be artificially introduced mutations or gain- or loss-of-function mutations. The mutations may include, but are not limited to, mutations in one of the catalytic domains (e.g., D10A and H840A in the RuvC and HNH catalytic domains, respectively). Further mutations have been characterized and may be used in one or more compositions of the disclosure. In one aspect of the disclosure, the dCas9 protein may be fused to a repressor or regulatory domains of other proteins, e.g., such as a transcriptional repression domain. In one aspect, of the disclosure, the transcriptional repression domain include, but is not limited to, ZIM3 Krüppel-associated box (ZIM3 KRAB domain), a transcription repression domain of methyl-CpG binding protein 2 (MeCP2 domain), and/or an interaction domain of Friend of GATA1 (FOG1 domain). The interaction domain of FOG1 comprises a repression domain of FOG1, the N-terminal of FOG1, and/or the N-terminal 45 residues of FOG1 (e.g., residues 1-45 of FOG1). Other aspects of the disclosure relate to the dCas 9 protein being fused to domains which include but are not limited to a transcriptional activator, repressor, a recombinase, a transposase, a histone remodeler, a demethylase, a DNA methyltransferase, a cryptochrome, a light inducible/controllable domain or a chemically inducible/controllable domain.

In one embodiment, the dCas9 protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:16; and/or ZIM3 KRAB domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:14; and/or MeCP2 domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:15; and/or FOG1 domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 17; and/or mTagBFP comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:18; and/or NLS comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:19; and/or NLS comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:20; and/or linker comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 21; and/or linker comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:22; and/or linker comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:23.

In certain embodiments, the dCas9 protein comprises one or more mutations selected from the group consisting of: D10A, E762A, H840A, N854A, N863A and D986A; and/or the one or more mutations is in a RuvC1 or HNH domain or is a mutation as otherwise as discussed herein (e.g., reference to SEQ ID NO:54). Cas9 sequences and structures from different species are known in the art (see e.g., Jinek et al. Science. 2012; see also SEQ ID NOs: 54-57). In some embodiments, the dCas9 has one or more mutations in a catalytic domain, wherein when transcribed, the tracr mate sequence hybridizes to the tracr sequence and the guide sequence directs sequence-specific binding of a CRISPR complex (e.g., a dCas9-gRNA complex) to the target sequence, and wherein the fusion protein comprises two or more functional domains. In some embodiments, the two or more functional domains include a transcriptional repression domain, for example, a ZIM3 Krüppel-associated box domain (ZIM3 KRAB domain), and/or a transcription repression domain of methyl-CpG binding protein 2 (MeCP2 domain), and/or an interaction domain of Friend of GATA1 (FOG1 domain). In some embodiments, the fusion protein comprises dCas9, ZIM3 Krüppel-associated box (ZIM3 KRAB domain), a transcription repression domain of methyl-CpG binding protein 2 (MeCP2 domain), and an interaction domain of Friend of GATA1 (FOG1 domain). In some embodiments, the fusion protein comprises additional functional domain, a fluorescent marker (FM) (e.g., mTagBFP, BFP, YFP, RFP, GFP, and eGFP). The FM may be used to improve repression by increasing spacing between the domains or increasing flexibility of the other attached functional domains. Moreover, the fluorescent marker may help track dCas9 expression and nuclear localization. Not wishing to be bound by any theory, the KRAB domain achieves repression in association with recruitment of the KAP1 co-repressor complex which contains the histone methyltransferase SETDB1, initiating tri-methylation of H3K9. The KRAB domain may act through: heterochromatin protein 1 (HP1), histone deacetylases, and/or SETDB1 responsible for methylation of H3K9. The transcription repression domain of MeCP2 binds to a different set of transcriptional regulators including the DNA methyltransferase DNMT1 and the SIN3A-histone deacetylase corepressor complex. The transcription repression domain of MeCP2 may act through: DNA methyltransferase DNMT1 and/or SIN3A-histone deacetylase corepressor complex. In addition, the N-terminal 45 residues of Friend of GATA-1 (FOG1 domain) has been shown to be associated with acquisition of H3K27me3 and loss of histone acetylation. N-terminal 45 residues of Friend Of GATA1 (FOG1 domain) may act through: histone deacetylation and/or recruitment of the PRC2 responsible for methylation of H3K27.

In one aspect, the CRISPR/Cas9-based system may include a dCas protein and two or more functional domains, or a nucleic acid encoding a fusion protein comprising a dCas protein and two or more functional domains. In one embodiment, the dCas protein and the two or more functional domains are linked covalently. In one embodiment, the two or more functional domains are fused in tandem to the dCas protein directly. In one embodiment, the two or more functional domains are covalently fused to the dCas protein indirectly, e.g., via a linker, a peptide, a NLS, or via an additional functional domain(s). In one embodiment, the two or more functional domains are at the N-terminus and/or C-terminus of the dCas protein. In one embodiment, the dCas protein and the two or more functional domains are linked in tandem. In one embodiment, a nucleic acid encoding a dCas protein is operably linked to two or more functional domains. In one embodiment, the dCas protein and the modulator of gene expression are fused to at least one fluorescent marker (FM). In one embodiment, the at least one FM may bring the dCas protein and the two or more functional domains into close proximity.

In one aspect, the use of fusion proteins comprising a dCas9 protein linked directly or indirectly to (or fused) to two or more repressor domains and nucleic acid molecules coding therefor in a method of repressing expression of a gene in a subject. In one embodiment, a composition comprising the fusion protein according to the present disclosure or the polynucleotide encoding the fusion protein, and one or more gRNAs that bind the fusion protein are administered to a subject. In one embodiment, the one or more gRNAs comprises a sequence that has sufficient complementarity with a target polynucleotide sequence. In one embodiment, the one or more gRNAs are capable of hybridizing with the target sequence. In one embodiment, the composition is packaged in a viral vector. In one embodiment, the viral vector is a lentiviral vector. In one embodiment, the viral vector is an adeno-associated virus (AAV) vector.

In one aspect, a viral vector comprising the polynucleotide according to the present disclosure; optionally further comprising one or more gRNAs that guide or direct the fusion protein to a target gene. In one embodiment, the viral vector is a lentiviral vector. In some embodiments, a vector encodes the fusion protein in any one the preceding aspects and/or embodiments comprising one or more nuclear localization sequences (NLSs), such as about or more than about 1, 2, 3, 4, 5, 6, 7, 8, 9, 10, or more NLSs. In some embodiments, the fusion protein comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In a preferred embodiment of the present disclosure, the fusion protein comprises 3 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In one embodiment, an NLS is linked adjacent to ZIM3 KRAB domain, and/or an NLS is linked adjacent to MeCP2 domain, and/or an NLS is linked adjacent to FOG1 domain. In some embodiments, one or more NLSs are linked adjacent to ZIM3 KRAB domain, MeCP2 domain, or FOG1 domain directly or indirectly (e.g., via a linker). Non-limiting examples of NLSs include an NLS sequence derived from the NLS of the SV40 virus large T-antigen, having the amino acid sequence PKKKRKV (SEQ ID NO:19) and the NLS from nucleoplasmin (e.g., the nucleoplasmin bipartite NLS with the sequence KRPAATKKAGQAKKKK (SEQ ID NO:20)).

In one embodiment, the fusion protein is a ZIM3 KRAB-MeCP2-NLS-dCas9 fusion protein comprising from the N-terminus to the C-terminus: ZIM3 KRAB domain, MeCP2 domain, a NLS, and a dCas9 protein. In some embodiments, ZIM3 KRAB domain, MeCP2 domain, a NLS, and a dCas9 protein are fused directly or indirectly (e.g., via a linker, an additional NLS, and/or one or more peptides). In one embodiment, the fusion protein is a ZIM3 KRAB-NLS-dCas9-FOG1 fusion protein comprising from the N-terminus to the C-terminus: ZIM3 KRAB domain, a NLS, a dCas9 protein, and FOG1 domain. In some embodiments, ZIM3 KRAB domain, a NLS, a dCas9 protein, and FOG1 domain are fused directly or indirectly (e.g., via a linker, an additional NLS, and/or one or more peptides). In one embodiment, the fusion protein is a ZIM3 KRAB-NLS-dCas9-NLS-FOG1 fusion protein comprising from the N-terminus to the C-terminus: ZIM3 KRAB domain, a NLS, a dCas9 protein, a NLS, and FOG1 domain. In some embodiments, ZIM3 KRAB domain, a NLS, a dCas9 protein, a NLS, and FOG1 domain are fused directly or indirectly (e.g., via a linker, an additional NLS, and/or one or more peptides). In yet another embodiment, the fusion protein is a MeCP2-NLS-dCas9-FOG1 fusion protein comprising from the N-terminus to the C-terminus: MeCP2 domain, a NLS, a dCas9 protein, and FOG1 domain. In some embodiments, MeCP2 domain, a NLS, a dCas9 protein, and FOG1 domain are fused directly or indirectly (e.g., via a linker, an additional NLS, and/or one or more peptides). In one embodiment, the fusion protein is a MeCP2-NLS-dCas9-NLS-FOG1 fusion protein comprising from the N-terminus to the C-terminus: MeCP2 domain, a NLS, a dCas9 protein, a NLS, and FOG1 domain. In some embodiments, MeCP2 domain, a NLS, a dCas9 protein, a NLS, and FOG1 domain are fused directly or indirectly (e.g., via a linker, an additional NLS, and/or one or more peptides). In one embodiment, the fusion protein is a ZIM3 KRAB-MeCP2-NLS-dCas9-NLS-FOG1 fusion protein comprising from the N-terminus to the C-terminus: ZIM3 KRAB domain, MeCP2 domain, a NLS, a dCas9 protein, a NLS, and FOG1 domain. In some embodiments, ZIM3 KRAB domain, MeCP2 domain, a NLS, a dCas9 protein, a NLS, and FOG1 domain are fused directly or indirectly (e.g., via a linker, an additional NLS, and/or one or more peptides).

In one embodiment, the fusion protein is a ZIM3 KRAB-MeCP2-NLS-dCas9-NLS-FM-NLS-FOG1 fusion protein comprising from the N-terminus to the C-terminus: ZIM3 KRAB domain, MeCP2 domain, a NLS, a dCas9 protein, a NLS, FM, a NLS, and FOG1 domain. In some embodiments, ZIM3 KRAB domain, MeCP2 domain, a NLS, a dCas9 protein, a NLS, FM, a NLS, and FOG1 domain are fused directly or indirectly (e.g., via a linker, an additional NLS, and/or one or more peptides). The fluorescent marker (FM) can be any one of fluorescent markers known in the art (e.g., mTagBFP, RFP, BFP, and GFP).

In some embodiments, the fusion protein is a ZIM3 KRAB-MeCP2-NLS-dCas9-NLS-FOG1 fusion protein comprising from the N-terminus to the C-terminus: ZIM3 KRAB domain, MeCP2 domain, a first NLS, dCas9 protein, a second NLS, and a FOG1 domain. In some embodiments, ZIM3 KRAB domain, MeCP2 domain, a first NLS, dCas9 protein, a second NLS, and a FOG1 domain are fused directly or indirectly (e.g., via a linker, an additional NLS, and/or one or more peptides). In one embodiment, the fusion protein is a ZIM3 KRAB-MeCP2-NLS-dCas9-NLS-FM-NLS-FOG1 fusion protein comprising from the N-terminus to the C-terminus: ZIM3 KRAB domain, MeCP2 domain, a first NLS, dCas9 protein, a second NLS, a FM, a third NLS, and a FOG1 domain. In some embodiments, ZIM3 KRAB domain, MeCP2 domain, a first NLS, dCas9 protein, a second NLS, a FM, a third NLS, and a FOG1 domain are fused directly or indirectly (e.g., via a linker, an additional NLS, and/or one or more peptides).

In one embodiment, the fusion protein is encoded by a nucleic acid comprising a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 7, or a sequence having one, two, three, four, five or more substitutions, insertions, or deletions relative to SEQ ID NO:7. In one embodiment, the polynucleotide encoding the dCas9 protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:10; and/or the polynucleotide encoding ZIM3 KRAB domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:8; and/or the polynucleotide encoding MeCP2 domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 9; and/or the polynucleotide encoding FOG1 domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:11; and/or the polynucleotide encoding mTagBFP comprises a sequence having at least 95%, 96%, 97%, 98%, 99%, or 100% sequence identity to SEQ ID NO:12.

In one embodiment, the fusion protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 13, or an amino acid sequence having one, two, three, four, five or more amino acid substitutions, insertions, or deletions relative to SEQ ID NO:13. In one embodiment, the fusion protein comprises an amino acid sequence having 100% sequence identity to SEQ ID NO:13.

In some embodiments, the dCas9 protein is part of a fusion protein comprising the two or more repressor domains (e.g., more than 2, 3, 4, 5, 6, 7, 8, 9, 10, or more domains in addition to the dCas9 protein). The fusion protein of present disclosure may comprise any additional protein sequence, and optionally a linker sequence between any two domains. Examples of proteins and/or sequences that may be fused to a dCas9 protein include, without limitation, fluorescent markers (FMs), tags, reporter gene sequences, and protein domains having one or more of the following activities: methylase activity, demethylase activity, transcription activation activity, transcription repression activity, transcription release factor activity, histone modification activity, RNA cleavage activity, nucleic acid binding activity and based editing. Non-limiting examples of tags, fluorescent markers (FMs), and reporter genes that can be used in the present disclosure include, but are not limited to, histidine (His) tags, V5 tags, FLAG tags, influenza hemagglutinin (HA) tags, Myc tags, VSV-G tags, and thioredoxin (Trx) tags, glutathione-S-transferase (GST), horseradish peroxidase (HRP), chloramphenicol acetyltransferase (CAT) beta-galactosidase, beta-glucuronidase, luciferase, green fluorescent protein (GFP), enhanced green flurescent protein (eGFP), HcRed, DsRed, cyan fluorescent protein (CFP), yellow fluorescent protein (YFP), blue fluorescent protein (BFP), and mTagBFP. In one embodiment, the fluorescent marker is a mTagBFP and has an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:18.

In some embodiments, the fusion of the dCas9 protein and the two or more repressor domains is direct (i.e., without any additional amino acids residues between the fused polypeptides/peptides). In other embodiments, the dCas9 protein and the two or more repressor domains are separated by a linker. As used herein, the term “linker” refers to a polypeptide that serves to connect the dCas9 protein with the two or more repressor domains and/or other protein sequences/domains including fluorescent markers, tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. The length of a linker peptide can vary; for example, the length may be as few as one amino acid or more than one hundred amino acids. Non-limiting examples of linker peptides contemplated herein can include flexible linkers, such as Gly-Ser linkers, and other similar linkers. Such linkers can have the formula Gly(x)-Ser(y) in which x=1, 2, 3, 4, 5, 6, 7, 8, 9, or 10 and y=1, 2, 3, 4, 5, 6, 7, 8, 9, or 10. The Gly-Ser linker can be replicated n number of times [(Gly(x)-Ser(y))n], for example, wherein n=1, 2, 3, 4, 5, 6, 7, 8, 9, 10, 15, 20, 25, or 30. In one embodiment, a linker has a sequence comprising any one of SEQ ID NOs: 21-23. The use of flexible linkers may aid in reducing steric hindrance. Moreover, the two or more functional domains fused with flexible linkers may reach multiple potential sites of influence. This may lead to better repression.

A guide RNA (gRNA) is any polynucleotide sequence having sufficient complementarity with a target polynucleotide sequence to hybridize with the target sequence and direct sequence-specific binding of a CRISPR complex to the target sequence. gRNAs useful in the disclosed methods include those having a spacer sequence, a tracr mate sequence and a tracr sequence, with the spacer sequence being between about 16 to about 20 nucleotides in length and with the tracr sequence being between about 60 to about 500 nucleotides in length and with a portion of the tracr sequence being hybridized to the tracr mate sequence and with the tracr mate sequence and the tracr sequence being linked by a linker nucleic acid sequence of between about 4 to about 6 nucleotides. crRNA-tracrRNA fusions are contemplated as exemplary guide RNA. In some embodiments, to generate gRNAs that target specific genes of interest, one or more gRNA libraries containing targeting sequences can be screened according to the protocols described in Replogle et al. Nature Biotechnology. 2020 (see also Sanger Arrayed Whole Genome Lentiviral CRISPR Library by Sigma). In one embodiment, a specific gRNA is selected from the gRNA libraries.

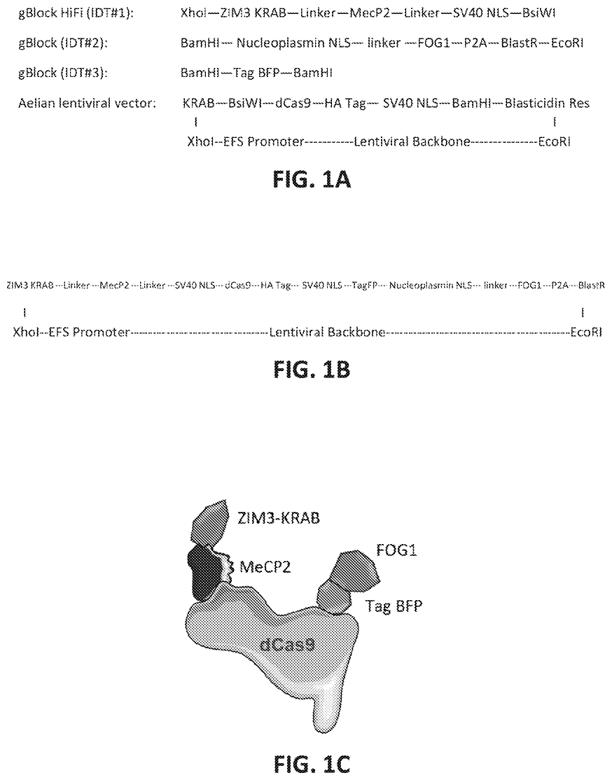

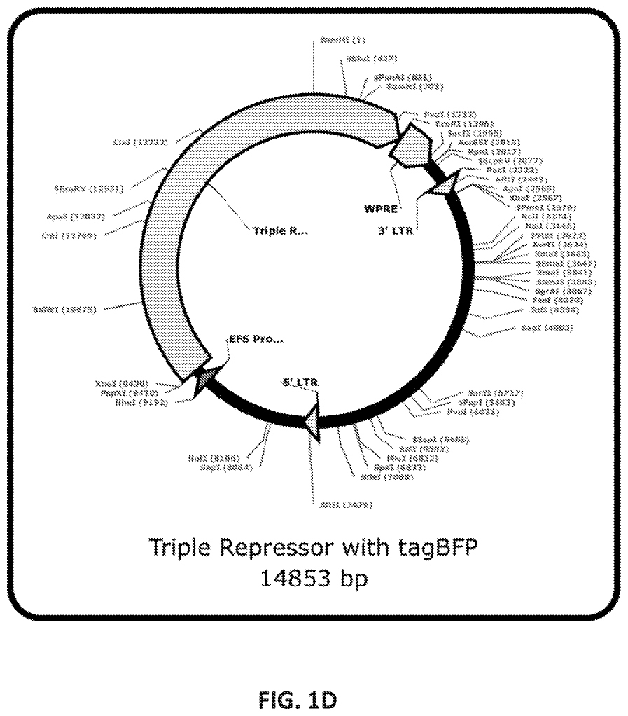

In one aspect, a pharmaceutical composion comprising the viral vector comprising the polynucleotide encoding the fusion proteins according to the present disclosure; and one or more gRNAs that bind the fusion protein is also provided herein. In one embodiment, the viral vector is a lentiviral vector. It is generally known to the one of ordinary skill in the art that large repressor domains need to be avoided as lentivirus payload size should not ideally exceed 10 kbps (see e.g., Sweeney and Vink, Molecular Therapy Methods & Clinical Development. 2021). Unexpectedly, the inventors of the present disclosure have developed a fully functional lentiviral vector having the payload size of about 14.9 kbps (FIG. 1D).

In one embodiment, a fusion protein for repressing expression of a gene is disclosed, comprising: a catalytically inactive Cas9 (dCas9) protein linked directly or indirectly to two or more repressor domains, wherein the two or more repressor domains selected from the group consisting of: (a) a Krüppel-associated box domain of ZIM3 gene (ZIM3 KRAB domain); (b) a transcription repression domain of methyl-CpG binding protein 2 (MeCP2 domain); and (c) an interaction domain of Friend of GATA1 (FOG1 domain). In one embodiment, the fusion protein further comprises one or more nuclear localization sequences (NLSs). In one embodiment, the fusion protein further comprises a fluorescent marker. In one embodiment, the fluorescent marker comprises at least one mTagBFP, BFP, YFP, RFP, and/or GFP. Preferably, the fluorescent marker comprises mTagBFP. The fusion protein according to any of the preceding embodiments, the dCas9 protein is linked directly or indirectly to ZIM3 KRAB domain and MeCP2 domain, ZIM3 KRAB a domain nd FOG1 domain, or MeCP2 domain and FOG1 domain, and/or the dCas9 protein is linked directly or indirectly to ZIM3 KRAB domain, MeCP2 domain, and FOG1 domain. In some embodiments, wherein the ZIM3 KRAB domain is linked to the N-terminus of the dCas9 protein, and/or wherein MeCP2 domain is linked to the N-terminus of dCas9, and/or wherein FOG1 domain is linked to the C-terminus of the dCas9 protein, or wherein the ZIM3 KRAB domain and MeCP2 domain are linked to the N-terminus of dCas9, and FOG1 domain is linked to the C-terminus of the dCas9 protein. In one embodiment, the fusion protein further comprises one or more linkers. Yet in another embodiment, wherein the dCas9 protein comprises a guide RNA (gRNA) binding domain, and/or wherein the dCas9 protein comprises at least one of a Rec1 domain, a bridge helix domain, or a protospacer adjacent motif interacting domain. In one embodiment, the dCas9 protein is a mutant of a wild-type Cas9 protein in which the Cas9 nuclease activity is inactivated. The dCas9 protein comprises one or more mutations that inactivate a Cas9 nuclease activity, the one or more mutations comprising a mutation in a RuvC1 domain and/or a mutation in a HNH domain, and/or wherein the one or more mutations comprises D10A and H840A mutations in the active site of the dCas9 protein.

In one aspect, a method of generating a stable cell and/or a stable cell expressing the fusion protein according to any one of the aspects and embodiments disclosed herein. In one embodiment, the method includes introducing into a culture of mammalian host cells, a viral vector comprising the polynucleotide encoding the fusion protein according to any one of preceding embodiments. In one embodiment, the viral vector is a lentiviral vector. In one embodiment, the viral vector is an adeno-associated virus (AAV) vector.

In one embodiment, the stable cell expresses the fusion protein, wherein the fusion protein is a ZIM3 KRAB-MeCP2-NLS-dCas9 fusion protein comprising from the N-terminus to the C-terminus: ZIM3 KRAB domain, MeCP2 domain, a NLS, and a dCas9 protein. In some embodiments, ZIM3 KRAB domain, MeCP2 domain, a NLS, and a dCas9 protein are fused directly or indirectly (e.g., via a linker, an additional NLS, and/or one or more peptides). In one embodiment, the fusion protein is a ZIM3 KRAB-NLS-dCas9-FOG1 fusion protein comprising from the N-terminus to the C-terminus: ZIM3 KRAB domain, a NLS, a dCas9 protein, and FOG1 domain. In some embodiments, ZIM3 KRAB domain, a NLS, a dCas9 protein, and FOG1 domain are fused directly or indirectly (e.g., via a linker, an additional NLS, and/or one or more peptides). In one embodiment, the fusion protein is a ZIM3 KRAB-NLS-dCas9-NLS-FOG1 fusion protein comprising from the N-terminus to the C-terminus: ZIM3 KRAB domain, a NLS, a dCas9 protein, a NLS, and FOG1 domain. In some embodiments, ZIM3 KRAB domain, a NLS, a dCas9 protein, a NLS, and FOG1 domain are fused directly or indirectly (e.g., via a linker, an additional NLS, and/or one or more peptides). In yet another embodiment, the fusion protein is a MeCP2-NLS-dCas9-FOG1 fusion protein comprising from the N-terminus to the C-terminus: MeCP2 domain, a NLS, a dCas9 protein, and FOG1 domain. In some embodiments, MeCP2 domain, a NLS, a dCas9 protein, and FOG1 domain are fused directly or indirectly (e.g., via a linker, an additional NLS, and/or one or more peptides). In one embodiment, the fusion protein is a MeCP2-NLS-dCas9-NLS-FOG1 fusion protein comprising from the N-terminus to the C-terminus: MeCP2 domain, a NLS, a dCas9 protein, a NLS, and FOG1 domain. In some embodiments, MeCP2 domain, a NLS, a dCas9 protein, a NLS, and FOG1 domain are fused directly or indirectly (e.g., via a linker, an additional NLS, and/or one or more peptides). In one embodiment, the fusion protein is a ZIM3 KRAB-MeCP2-NLS-dCas9-NLS-FOG1 fusion protein comprising from the N-terminus to the C-terminus: ZIM3 KRAB domain, MeCP2 domain, a NLS, a dCas9protein, a NLS, and FOG1 domain. In some embodiments, ZIM3 KRAB domain, MeCP2 domain, a NLS, a dCas9 protein, a NLS, and FOG1 domain are fused directly or indirectly (e.g., via a linker, an additional NLS, and/or one or more peptides). In one embodiment, the fusion protein is a ZIM3 KRAB-MeCP2-NLS-dCas9-NLS-FM-NLS-FOG1 fusion protein comprising from the N-terminus to the C-terminus: ZIM3 KRAB domain, MeCP2 domain, a NLS, a dCas9 protein, a NLS, FM, a NLS, and FOG1 domain. In some embodiments, ZIM3 KRAB domain, MeCP2 domain, a NLS, a dCas9 protein, a NLS, FM, a NLS, and FOG1 domain are fused directly or indirectly (e.g., via a linker, an additional NLS, and/or one or more peptides). In one embodiment, the stable cell includes a cell line having HEK293T cells, A549 cells, or K562 cells. In one embodiment, the FM comprises at least one of mTagBFP, BFP, YFP, RFP, GFP, and eGFP.

In some embodiments, the stable cell expresses the fusion protein, wherein the fusion protein is a ZIM3 KRAB-MeCP2-NLS-dCas9-NLS-FOG1 fusion protein comprising from the N-terminus to the C-terminus: ZIM3 KRAB domain, MeCP2 domain, a first NLS, dCas9 protein, a second NLS, and a FOG1 domain. In some embodiments, ZIM3 KRAB domain, MeCP2 domain, a first NLS, dCas9 protein, a second NLS, and a FOG1 domain are fused directly or indirectly (e.g., via a linker, an additional NLS, and/or one or more peptides). In one embodiment, the fusion protein is a ZIM3 KRAB-MeCP2-NLS-dCas9-NLS-FM-NLS-FOG1 fusion protein comprising from the N-terminus to the C-terminus: ZIM3 KRAB domain, MeCP2 domain, a first NLS, dCas9 protein, a second NLS, a FM, a third NLS, and a FOG1 domain. In some embodiments, ZIM3 KRAB domain, MeCP2 domain, a first NLS, dCas9 protein, a second NLS, a FM, a third NLS, and a FOG1 domain are fused directly or indirectly (e.g., via a linker, an additional NLS, and/or one or more peptides).

In one embodiment, the stable cell expresses the fusion protein, wherein the fusion protein is encoded by a nucleic acid comprising a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:7, or a sequence having one, two, three, four, five or more substitutions, insertions, or deletions relative to SEQ ID NO: 7. In one embodiment, the polynucleotide encoding the dCas9 protein comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 10; and/or the polynucleotide encoding ZIM3 KRAB domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:8; and/or the polynucleotide encoding MeCP2 domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:9; and/or the polynucleotide encoding FOG1 domain comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:11; and/or the polynucleotide encoding mTagBFP comprises a sequence having at least 95%, 96%, 97%, 98%, 99%, or 100% sequence identity to SEQ ID NO:12.

In one embodiment, the stable cell expresses the fusion protein, wherein the fusion protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:13, or an amino acid sequence having one, two, three, four, five or more amino acid substitutions, insertions, or deletions relative to SEQ ID NO: 13.

In one aspect, there is provided a method of repressing expression of a gene in a subject, comprising providing to the subject: (a) the fusion protein or the polynucleotide according to any one of the preceding embodiments; and (b) one or more gRNAs that direct the fusion protein or the polynucleotide to the gene. In one embodiment, the one or more gRNA comprises at least one sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to at least one of SEQ ID NOs: 24-32, or at least one sequence having one, two, three, four, five or more substitutions, insertions, or deletions relative to at least one of SEQ ID NOs: 24-32. In one embodiment, one or both of (a) and (b) are packaged in a viral vector, wherein (a) and (b) are packaged in the same viral vector, or wherein each of (a) and (b) is packaged in a separate viral vector. In one embodiment, the viral vector comprises a lentiviral vector. The method according to the present disclosure provides that the expression of the gene may be repressed at least about 50%, about 60%, about 70%, about 80%, about 90%, about 95%, and about 99% as compared to the expression of the gene in a wild-type control. Consistent with these embodiments, the gene is an endogenous gene of the subject, and the subject comprises adherent cells, suspension cells, tissues, animals, mammals, and humans.

In one aspect, a viral vector is provided and comprises (i) the polynucleotide encoding the fusion protein according to any one of preceding embodiments; and/or (ii) one or more gRNAs that direct the fusion protein or the polynucleotide to a gene of interest. In one embodiment, the one or more gRNAs include any polynucleotide sequence having sufficient complementarity with a target polynucleotide sequence to hybridize with the target sequence and direct sequence-specific binding of a CRISPR complex to the target sequence. In some embodiments, to generate gRNAs that target specific genes of interest, one or more gRNA libraries containing targeting sequences can be screened according to the protocols described in Replogle et al. Nature Biotechnology. 2020 (see also Sanger Arrayed Whole Genome Lentiviral CRISPR Library by Sigma). In one embodiment, a specific gRNA is selected from the gRNA libraries, e.g., Sanger Arrayed Whole Genome Lentiviral CRISPR Library. In other embodiment, the one or more gRNA comprises at least one sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to at least one of SEQ ID NOs: 24-30, or at least one sequence having one, two, three, four, five or more substitutions, insertions, or deletions relative to at least one of SEQ ID NOs: 24-32. The viral vector provided herein can include or is a lentiviral vector.

In one aspect, a pharmaceutical composition is provided and comprises a therapeutically effective amount of: (a) the polynucleotide encoding the fusion protein according to any one of the preceding embodiments; and (b) one or more gRNAs that bind the fusion protein or the polynucleotide encoding the fusion protein. In one embodiment, the one or more gRNAs include any polynucleotide sequence having sufficient complementarity with a target polynucleotide sequence to hybridize with the target sequence and direct sequence-specific binding of a CRISPR complex to the target sequence. In some embodiments, to generate gRNAs that target specific genes of interest, one or more gRNA libraries containing targeting sequences can be screened according to the protocols described in Replogle et al. Nature Biotechnology. 2020 (see also Sanger Arrayed Whole Genome Lentiviral CRISPR Library by Sigma). In one embodiment, a specific gRNA is selected from the gRNA libraries, e.g., Sanger Arrayed Whole Genome Lentiviral CRISPR Library. In other embodiment, the one or more gRNA comprises at least one sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to at least one of SEQ ID NOs: 24-30, or at least one sequence having one, two, three, four, five or more substitutions, insertions, or deletions relative to at least one of SEQ ID NOs: 24-32. In one embodiment, each of (a) and (b) is packaged in a separate viral vector or both (a) and (b) are packaged in the same viral vector. The viral vector comprises or is a lentiviral vector.

In one aspect, a pharmaceutical composition for use in a method of repressing expression of a gene in a subject, comprising a therapeutically effective amount of: (a) the polynucleotide encoding the fusion protein according to any one of the preceding embodiments; and (b) one or more gRNAs that bind the fusion protein or the polynucleotide encoding the fusion protein. In one embodiment, the one or more gRNAs include any polynucleotide sequence having sufficient complementarity with a target polynucleotide sequence to hybridize with the target sequence and direct sequence-specific binding of a CRISPR complex to the target sequence. In some embodiments, to generate gRNAs that target specific genes of interest, one or more gRNA libraries containing targeting sequences can be screened according to the protocols described in Replogle et al. Nature Biotechnology. 2020 (see also Sanger Arrayed Whole Genome Lentiviral CRISPR Library by Sigma). In one embodiment, a specific gRNA is selected from the gRNA libraries, e.g., Sanger Arrayed Whole Genome Lentiviral CRISPR Library. In other embodiment, the one or more gRNA comprises at least one sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to at least one of SEQ ID NOs: 24-30, or at least one sequence having one, two, three, four, five or more substitutions, insertions, or deletions relative to at least one of SEQ ID NOs: 24-32. In one embodiment, each of (a) and (b) is packaged in a separate viral vector or both (a) and (b) are packaged in the same viral vector. The viral vector comprises or is a lentiviral vector. In one embodiment, the pharmaceutical composition represses the expression of the gene at least about 50%, about 60%, about 70%, about 80%, about 90%, about 95%, and about 99% as compared to the expression of the gene of a normal subject. In one embodiment, the gene is an endogenous gene of the subject. In one embodiment, the subject includes, but is not limited to, animals, mammals, and humans.

Pharmaceutical compositions may be administered by injection or continuous infusion (examples include, but are not limited to, intravenous, intraperitoneal, intradermal, subcutaneous, intramuscular, intraocular, and intraportal). In one embodiment, the composition is suitable for intravenous, intraperitoneal, intradermal, or subcutaneous administration. The pharmaceutical composition may be included in a kit containing the antigen binding protein together with other medicaments, and/or with instructions for use. For convenience, the kit may comprise the reagents in predetermined amounts with instructions for use. The kit may also include devices used for administration of the pharmaceutical composition.

### Definitions

Unless defined otherwise, all technical and scientific terms used herein have the same meaning as is commonly understood by one of skill in the art to which this disclosure belongs. All patents and publications referred to herein are incorporated by reference in their entirety.

The term “about” or “approximately” can mean within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “Cas9 protein” may refer to a Cas9 enzyme. Cas9 protein is an endonuclease that cleaves nucleic acid and is encoded by the CRISPR loci and is involved in the Type II CRISPR system. The Cas9 protein may be from any bacterial or archaea species, such as Streptococcus pyogenes. Sequences and structures of Cas9 from different species are known in the art (see, e.g., Jinek et al. Science. 2012; see also SEQ ID NOs: 54-57).

The terms “catalytically inactive Cas9” and “dCas9” as used interchangeably herein refer to a CRISPR/Cas protein variant or mutant that lacks endonuclease activity (i.e., no ability to cleave double stranded DNA) but is capable of binding to DNA. For example, catalytically-inactive Cas9 mutants have been generated through incorporation of various mutations (e.g., D10A and H840A) mutations (Jinek et al. Science. 2012; Qi et al. Cell. 2013).

The term “CRISPR/Cas system” refers to a widespread class of bacterial defense systems against foreign nucleic acid. CRISPR/Cas systems are found in a wide range of bacterial and archaeal organisms. CRISPR/Cas systems include, but are not limited to, type I, II, III, IV, V and VI sub-types. Type II CRISPR/Cas systems utilize the RNA-mediated nuclease, Cas9 protein in complex with RNA to recognize and cleave foreign nucleic acid. Type V CRISPR/Cas systems utilize Cas12a protein. Since the structures of type II and V CRISPR/Cas systems are relatively simple, these systems have been widely used in bacteria. For example, type II CRISPR/Cas systems only require crRNA, tracrRNA, and Cas9 protein while type V CRISPR/Cas systems only require crRNA and Cas 12a protein (see e.g., Liu et al. Microb Cell Fact. 2020). Suitable dCas protein can be derived from a wild type Cas protein. The dCas protein can be from type I, II, III, IV, V, or VI CRISPR-Cas systems.

The term “domain” refers to a folded polypeptide structure that retains its tertiary structure independent of the rest of the polypeptide. Generally, domains are responsible for discrete functional properties of polypeptides and in many cases may be added, removed or transferred to other polypeptides without loss of function of the remainder of the protein and/or of the domain.

The term “endogenous gene” as used herein refers to a gene that originates from within an organism, tissue, or cell. An endogenous gene is native to a cell, which is in its normal genomic and chromatin context, and which is not heterologous or foreign to the cell. Such cellular genes include, e.g., animal genes, plant genes, bacterial genes, fungal genes, and mitochondrial genes. An “endogenous target gene” as used herein refers to an endogenous gene that is targeted by an optimized gRNA and CRISPR/Cas9-based system or dCas9-based system.

The term “fusion protein” refers to a chimeric protein created through the covalent in tandem joining of two or more genes, directly or indirectly, that originally coded for separate proteins. In some embodiments, the translation of the fusion gene results in a single polypeptide with functional properties derived from each of the original proteins.

The term “genetic construct” or “construct” refers to the DNA or RNA molecules that comprise a nucleotide sequence that encodes a protein. The coding sequence includes initiation and termination signals operably linked to regulatory elements including a promoter and polyadenylation signal capable of directing expression in cells.

The terms “guide RNA,” “gRNA,” “single gRNA,” “small gRNA,” and “sgRNA” as used interchangeably herein refer to a short synthetic RNA composed of a “scaffold” sequence necessary for Cas9-binding and a user-defined “spacer,” “targeting sequence,” “protospacer-targeting sequence,” or “segment” which defines the genomic target to be modified. In some embodiments, the gRNA is a fusion of two noncoding RNAs: a crRNA and a tracrRNA. The gRNA may target any desired DNA sequence by exchanging the sequence encoding a 20 bp protospacer which confers targeting specificity through complementary base pairing with the desired DNA target. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to cleave the target nucleic acid. The term “target region”, “target sequence” or “protospacer” as used interchangeably herein refers to the region of the target gene to which the CRISPR/Cas9-based system targets. The CRISPR/Cas9-based systems or dCas9-based systems may include one or more gRNAs, wherein the gRNAs target different DNA sequences. In some embodiments, the target sequence or protospacer is followed by a protospacer adjacent motif (PAM) sequence at the 3′ end of the protospacer.

The term “expression of a gene” or “gene expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” The process of gene expression is used by all known life—eukaryotes (including multicellular organisms), prokaryotes (bacteria and archaea) and viruses to generate functional products to survive.

The term “linker” or “linker peptide” refers to a polypeptide that serves to connect the CRISPR/Cas or dCas9 protein with the repressors or repressor domains of a fusion protein. The length of a linker peptide can vary; for example, the length may be as few as one amino acid or more than one hundred amino acids. Non-limiting examples of linker peptides used herein include linkers comprising at least one of glycine, serine, alanine, glutamic acid, and/or phenylalanine. Such linkers can have the formula Gly(x)-Ser(y) in which (x)=1, 2, 3, 4, 5, 6, 7, 8, 9, or 10 and (y)=1, 2, 3, 4, 5, 6, 7, 8, 9, or 10.

The term “modulate” as used herein may include altering of an activity, such as to regulate, down regulate, upregulate, reduce, inhibit, increase, decrease, deactivate, or activate.

The terms “non-naturally occurring” and “engineered” are used interchangeably and indicate human involvement. The terms, when referring to nucleic acid molecules or polypeptides mean that the nucleic acid molecule or the polypeptide is at least substantially free from at least one other component with which they are naturally associated in nature and as found in nature.

The terms “normal gene” and “wild-type gene” as used interchangeably herein refer to a gene that has not undergone a change, such as a loss, gain, or exchange of genetic material. The normal gene undergoes normal gene transmission and gene expression. The term “wild-type” (wt) refers to a gene or gene product which has the characteristics of that gene or gene product when isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants may be isolated, which are identified by the acquisition of altered characteristics when compared to the wild-type gene or gene product.

The term “operably linked” as used herein means that expression of a gene is under the control of a promoter with which it is spatially connected. A promoter may be positioned 5′ (upstream) or 3′ (downstream) of a gene under control. The distance between the promoter and a gene may be approximately the same as the distance between that promoter and the gene it controls in the gene from which the promoter is derived. As is known in the art, variation in this distance may be accommodated without loss of promoter function.

The term, “percent identity” or “% identity” or “sequence identidy” between a query nucleic acid sequence/amino acid sequence and a subject nucleic acid sequence/amino acid sequence is the “Identities” value, expressed as a percentage, that is calculated using a suitable algorithm (e.g., BLASTN, FASTA, Needleman-Wunsch, Smith-Waterman, LALIGN, or GenePAST/KERR) or software (e.g., DNASTAR Lasergene, GenomeQuest, EMBOSS needle or EMBOSS infoalign), over the entire length of the query sequence after a pair-wise global sequence alignment has been performed using a suitable algorithm (e.g., Needleman-Wunsch or GenePAST/KERR) or software (e.g., DNASTAR Lasergene or GenePAST/KERR). Importantly, a query nucleic acid sequence/amino acid sequence may be described by a nucleic acid sequence/amino acid sequence disclosed herein, in particular in one or more of the claims. The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. In the case of nucleic acid sequences, such alterations include at least one nucleotide residue deletion, substitution or insertion, wherein said alterations may occur at the 5′- or 3′-terminal positions of the query sequence or anywhere between those terminal positions, interspersed either individually among the nucleotide residues in the query sequence or in one or more contiguous groups within the query sequence. In the case of amino acid sequences, such alterations include at least one amino acid residue deletion, substitution (including conservative and non-conservative substitutions), or insertion, wherein said alterations may occur at the amino- or carboxy-terminal positions of the query sequence or anywhere between those terminal positions, interspersed either individually among the amino acid residues in the query sequence or in one or more contiguous groups within the query sequence.

The terms “polynucleotide,” “nucleotide,” “nucleotide sequence,” “nucleic acid,” and “oligonucleotide” are used interchangeably and refer to a polymeric form of nucleotides of any length, either deoxyribonucleotides or ribonucleotides, or analogs thereof.

The term “promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs, or anywhere in the genome, from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, hormones, toxins, drugs, pathogens, metal ions, or inducing agents. Representative examples of promoters include the bacteriophage T7 promoter, bacteriophage T3 promoter, EF1 promoter, PGK promoter, CAG promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

The term “protospacer adjacent motif” or “PAM” as used herein refers to a DNA sequence immediately following the DNA sequence targeted by the Cas9 in the CRISPR bacterial adaptive immune system. PAM is a component of the invading virus or plasmid, but is not a component of the bacterial CRISPR locus. Cas9 will not successfully bind to or cleave the target DNA sequence if it is not followed by the PAM sequence. PAM is an essential targeting component (not found in bacterial genome) which distinguishes bacterial self from non-self DNA, thereby preventing the CRISPR locus from being targeted and destroyed by nuclease. The terms “protospacer sequence” and “protospacer segment” as used interchangeably herein refer to a DNA sequence targeted by the Cas9 nuclease in the CRISPR bacterial adaptive immune system. In the CRISPR/Cas9 system, the protospacer sequence is typically followed by a protospacer-adjacent motif (PAM); the PAM is at the 5′ end. The terms “protospacer-targeting sequence” and “protospacer-targeting segment” as used interchangeably herein refer to a nucleotide sequence of a gRNA that corresponds to the protospacer sequence and facilitates targeting of the CRISPR/Cas9-based system to the protospacer sequence.

The term “selectable marker” refers to a gene that will help select cells actively expressing an inserted gene (e.g., a transgene). Examples of suitable selection markers may include enzymes encoding resistance to an antibiotic (i.e., an antibiotic resistance gene), e.g., kanamycin, neomycin, puromycin, hygromycin, blasticidin, or zeocin. Other examples of suitable selection markers include fluorescent markers, e.g., mTagBFP, blue fluorescent protein (BFP), green fluorescent protein (GFP), red fluorescent protein (RFP), or yellow fluorescent protein (YFP).

The term “stably transfected” refers to cell lines which are able to pass introduced retroviral genes to their progeny (i.e., daughter cells), either because the transfected DNA has been incorporated into the endogenous chromosomes or via stable inheritance of exogenous chromosomes. The term “stable transfectant” refers to a cell, which has stably integrated foreign DNA into its genomic DNA.

The term “target gene” as used herein refers to a nucleotide sequence encoding a known or putative gene product. The target gene may be a mutated gene involved in a genetic disease or disorder.

The term “therapeutically effective amount” or “therapeutic effective dose” refers to an amount or dose of a fusion protein, polypeptide, nucleic acid, lentivirus particle(s), or virion(s) capable of producing sufficient amounts of a desired protein or RNA to modulate the expresion of a gene in a desired manner, thus providing a palliative tool for clinical intervention. In some embodiments, a therapeutically effective amount or dose of a transfected fusion protein, polypeptide, nucleic acid, lentivirus particle(s), or virion(s) as described herein is enough to confer suppression of a gene targeted by the fusion protein/gene therapy construct.

The term “transcriptional start site” or “TSS” as used interchangeably herein refers to the first nucleotide of a transcribed DNA sequence where RNA polymerase begins synthesizing the RNA transcript.

The terms “transfection”, “transformation” and “transduction” as used herein, may be used to describe the insertion of the non-mammalian or viral vector into a target cell. Insertion of a vector is usually called transformation for bacterial cells and transfection for eukaryotic cells, although insertion of a viral vector may also be called transduction. The skilled person will be aware of the different non-viral transfection methods commonly used, which include, but are not limited to, the use of physical methods (e.g., electroporation, cell squeezing, sonoporation, optical transfection, protoplast fusion, impalefection, magnetofection, gene gun or particle bombardment), chemical reagents (e.g., calcium phosphate, highly branched organic compounds or cationic polymers) or cationic lipids (e.g., lipofection). Many transfection methods require the contact of solutions of plasmid DNA to the cells, which are then grown and selected for a marker gene expression.

The term “transgene” as used herein refers to a gene or genetic material containing a sequence that has been isolated from one organism and is introduced into a different organism. Additionally, the term “transgene” may also refer to a gene or genetic material that is chemically synthesized and introduced into an organism. This non-native segment of DNA may retain the ability to produce RNA or protein in the transgenic organism.

The term “vector” or “nucleic acid vector” refers to a vehicle which is able to artificially carry foreign (i.e., exogenous) genetic material into another cell, where it can be replicated and/or expressed. Examples of vectors include viral vectors, such as retroviral, adeno-associated virus (AAV), and lentiviral vectors, which are of particular interest in the present application. Lentiviral vectors, such as those based upon Human Immunodeficiency Virus Type 1 (HIV-1) are widely used as they are able to integrate into non-proliferating cells. Viral vectors can be made replication defective by splitting the viral genome into separate parts, e.g., by placing on separate plasmids. Adeno-associated virus (AAV) vectors can also be used for gene delivery of CRISPR-Cas9 components for in vivo studies and therapeutic applications.

### EXAMPLES

### Example 1: Development of Triple Inhibitory Domain CRISPRi System

In the example shown in FIG. 1A-1C, AELIAN lentiviral vector (to remove the KRAB) and IDT #1 were digested with XhoI (NEB Cat #R0146S) and BsiWI (NEB Cat # R3553S) and ligated to form Construct #1, according to the manufacturers' protocols. Briefly, Zim3 KRAB and Kox1 KRAB have been cloned in AELIAN dCas9 lentiviral vector. After digestion of AELIAN EFS ZIM3 KRAB dCas9 P2A Blast lentiviral vector with restriction enzymes (using standard protocols from NEB), the digested fragments were dephosphorylated using antarctic phosphatase (NEB #M0289). The fragment of interest comprising the vector (12678 bps) was isolated from the smaller fragment (312 bps) by running the digested and dephosphorylated DNA on an agarose gel and performing gel extraction and purification (QIAQUICK Gel Extraction Kit, Cat #28704, see e.g., Qiagen's protocol). IDT #1 was digested with the restriction enzymes above and the fragment was cleaned up using Qiagen's QIAQUICK PCR purification kit (Cat #28104). Ligation between the fragments was conducted using NEB's T4 DNA Ligase (M0202T). The ligated products was cloned by transforming ONE SHOT STBL3 chemically competent E. coli (ThermoFisher Scientific Cat #C737303). Clones were picked and amplified using miniprep colony culture and plasmid DNA purified using the QIAPREP Spin Miniprep Kit (Qiagen Cat #27106). The insertion of IDT #1 fragment into the dCas9 lentiviral vector was validated using restriction digestion with XhoI and BsiWI. The ligation products generated about 1250 bps and about 12.7 kbps fragments.

Construct #1 (to remove the small fragment containing blasticidin resistance gene) and IDT #2 were digested with EcoRI (NEB Cat #R3101S) and BamHI (NEB Cat # R3136S) and ligated to form Construct #2, according to the manufacturers' protocols (FIG. 1A). Briefly, after digestion of Construct #1 plasmid with restriction enzymes (using standard protocols from NEB), the fragments were dephosphorylated using antarctic phosphatase (NEB #M0289). The fragment of interest comprising the vector (13458 bps) was isolated from the smaller fragment (465 bps) by running the digested and dephosphorylated DNA on an agarose gel and performing gel extraction and purification (QIAQUICK Gel Extraction Kit, Cat #28704, see e.g., Qiagen's protocol). IDT #2 was digested with the restriction enzymes above and the fragment was cleaned up using Qiagen's QIAQUICK PCR purification kit (Cat #28104). Ligation between the fragments was performed using NEB's T4 DNA Ligase (M0202T). The ligated products were cloned by transforming ONE SHOT STBL3 chemically competent E. coli (ThermoFisher Scientific Cat #C737303). Clones were picked and cultured. Plasmid DNA was isolated and purified using the QIAPREP Spin Miniprep Kit (Qiagen Cat #27106). 10 colonies were picked and miniprep DNA was analyzed. Clones 2 to 8 demonstrated the correct restriction digest pattern when run on an agarose gel (see the image below). DNA from Clone 2 to Clone 6 were sequenced. Sequence validation was done using Sanger sequencing (IRMS request ID: SC448AC). Clone 3 and Clone 5 were found to contain the correct sequences. DNA from both the clones (containing the desired sequence) were combined to form Construct #2.

Construct #2 and mTagBFP were digested with BamHI and ligated to form the final construct (Construct #3), according to the manufacturers' protocols (FIG. 1A and 1B). Briefly, after digestion of Construct #2 plasmid with restriction enzymes (using standard protocols from NEB), the fragments were dephosphorylated using antarctic phosphatase (NEB #M0289). The linearized plasmid was isolated from circular uncut plasmid by running the digested and dephosphorylated DNA on an agarose gel and performing gel extraction and purification (QIAQUICK Gel Extraction Kit, Cat #28704, see e.g., Qiagen's protocol). IDT #3 fragment (mTagBFP) was digested with BamHI and the fragment was cleaned up using Qiagen's QIAQUICK PCR purification kit (Cat #28104). Ligation between the fragments was conducted using NEB's T4 DNA Ligase (M0202T). The ligated products were cloned by transforming ONE SHOT STBL3 chemically competent E. coli (ThermoFisher Scientific Cat #C737303). Clones were picked and miniprep colony culture initiated. QIAPREP Spin Miniprep Kit (Qiagen Cat #27106) was used to purify plasmid DNA. The insertion of mTagBFP fragment into the vector comprising IDT #1 and IDT #2 fragments was validated using restriction digestion with PshAI (NEB Cat # R0593S) and EcoRI. The ligation product generated fragments of 765 bps and 14088 bps size after restriction digestion with the above enzymes. 12 colonies were picked and miniprep DNA was analyzed. Clone 1 and clones 6 to 11 demonstrated the correct restriction bands on an agarose gel. DNA from Clone 9 and Clone 11 were arbitrarily selected and were sequenced and validated. In conclusion, a rational dCas9 repressor lentiviral plasmid based on complementary mechanisms of repression (Tri methylation of H3K27+Tri methylation of H3K9+HDAC promotion+DNA Methylation) was successfully generated using standard molecular cloning techniques. This final CRISPRi construct comprising triple repressor domains and a mTagBFP (also referred to as “Triple Repressor” or “Triple Rep”) was used in subsequent examples.

### Example 2: Generation of Lentiviruses

HEK293T cells (Takara, Cat #632180) were grown in D-MEM medium plus glutamine supplemented with 10% FBS without antibiotics and expand until it reached sufficient cell counts to package at the scale desired. Twenty four (24) hours prior to transfection, plate 6 million HEK293T cells per 75 cm² flask and use 10 ml of media per plate. 15 μg Ready-to-use Lentiviral Packaging Plasmid Mix (Cellecta, Cat. #CPCP-K2A) and 3 μg plasmid Lentiviral construct were mixed in a sterile polypropylene tube. For each flask, 8 ml of complete medium was added with serum and antibiotics 30-60 minutes before transfection. 18 μg of DNA were diluted into 750 μl of serum-free DMEM. 54 μl of CALFECTIN reagent was added immediately and directly into the 750 μl diluted DNA solution and incubated for 10-15 minutes at room temperature to allow CALFECTIN/DNA complexes to form. 750 μl of CALFECTIN/DNA mixture were added drop-wise onto the medium in each flask and homogenized by gently swirling the flask. CALFECTIN/DNA complex-containing medium was removed and replaced with 11 ml of fresh complete serum/antibiotics medium 4 hours post transfection. The viral supernatant (10 ml) was collected after 24 hours and 48 hours.

Additionally, triple repressor viruses were concentrated using Takara's LENTI-X CONCENTRATOR (Cat. Nos. 631231 & 631232). Viral supernatant is collected from virus-producing cell line and centrifuged to remove cells and debris. It is then mixed with the LENTI-X CONCENTRATOR and incubated at 4° C. for 30 minutes to overnight. The mixture is then centrifuged at low speed to obtain a high-titer virus-containing pellet which can then easily be resuspended and used for transduction of intended target cells.

### Example 3: Generation of Cell Lines

Polyclonal KRAB (KOX1, ZIM3, and UCOE)-based CRISPRi lines were generated by post transduction blasticidin selection. Polyclonal triple repressor-based CRISPRi lines were also generated by post transduction blasticidin selection and FACS of BFP positive cells. For example, five dCas9 HEK293T lines: ZIM3 KRAB, KOX1 KRAB, UCOE KRAB, Triple Repressor clone 11 and clone 9; five dCas9 A549 lines: ZIM3 KRAB, KOX1 KRAB, UCOE KRAB, Triple Repressor clone 11 and clone 9; and two dCas9 K562 lines: ZIM3 KRAB, and Triple Repressor clone 11 were generated using this protocol. Briefly, in a 25 cm2 flask 250,000 cells were seeded with 4 ml of media (DMEM with 10% FBS). Cells were transduced with 200 μl of unconcentrated virus (ZIM3 KRAB, KOX1 KRAB, UCOE KRAB) or 200 μl of concentrated virus (Triple Repressor clone 11 and clone 9). Polybrene at a concentration of 8 μg/ml of media was added. Cells were selected with blasticidin antibiotic (20 μg/ml for A549 and K562, and 10 μg/ml for HEK293T) 3 days after transduction. The triple repressor cells lines were sorted for BFP to remove the cells that were not expressing the full construct due to recombination.

### Example 4: CRISPRi Reporter Assay (CRISPRITEST)

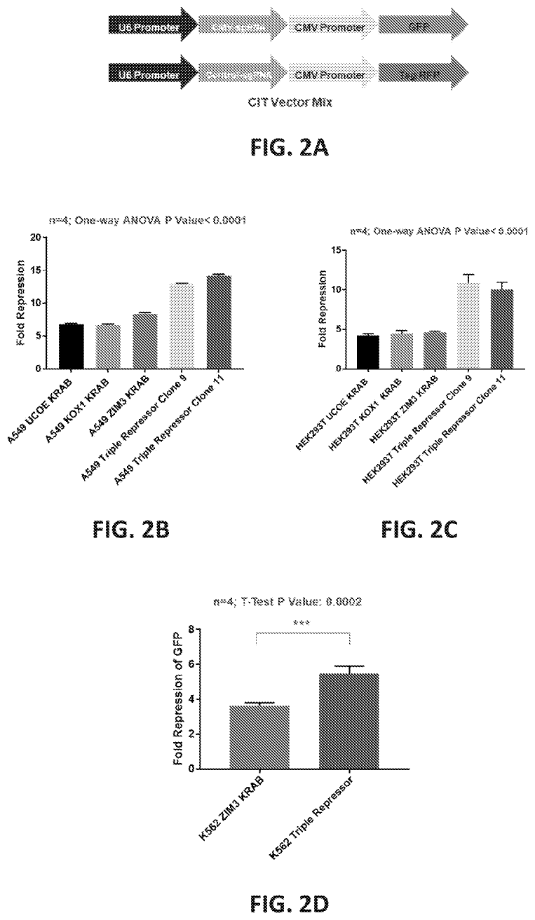

CRISPRi reporter assay was conducted according to the manufacturer's protocol (see e.g., CRISPRITEST Functional dCas9-Repressor Assay Kit by CELLCTA,). Briefly, the CiT virus mix contains two premixed lentivectors: (1) a vector expressing GFP from the CMV promoter and a U6-driven sgRNA targeting the CMV-GFP transcription start site (2) a vector expressing RFP from the CMV promoter and a U6-driven non-targeting gRNA. The mean GFP and RFP fluorescent values are then used to calculate dCas9-Repressor activity in dCAs9-Repressor cells (FIG. 2A). Parental and dCas9 KRAB expressing cells (ZIM3 KRAB, KOX1 KRAB, UCOE KRAB, Triple Repressor clone 11 and clone 9) were transduced with CiT virus mix. The transduced cells were grown for 3 days. At day 4, the transduced cells were analyzed by flow cytometry (Channel 1: excitation 488 nM, emission 530/20 nm (GFP); Channel 2: excitation 561 nM, emission 590/20 nm (RFP)).

In the example shown in FIGS. 2B-2C, two adherent cell lines (A549 and HEK293T) were selected. Reporter repression caused by both the triple repressor dCas9 clones were statistically superior (>2× higher repression) than any of the KRAB-based CRISPRi systems (ZIM3 KRAB, KOX1 KRAB, and UCOE KRAB). In A549 cells, although Zim3 KRAB may be considered the most potent KRAB-based system, both the triple repressor dCas9 clones exhibited significant increase in fold repression when compared to all KRAB-based systems (FIG. 2B). In HEK293T, there is no difference among the KRAB-based systems. Again, both the triple repressor dCas9 clones exhibited significant increase in fold repression when compared to all KRAB-based systems (FIG. 2C). Since both clone 9 and clone 11 performed equally well, clone 11 triple repressor system was chosen and Zim3 KRAB was chosen as a representative of the KRAB-based systems for all subsequent experiments. In the example shown in FIG. 2D, triple repressor also exhibited significant increase in fold repression (>1.5× higher repression) when compared to that of Zim3 KRAB CRISPRi system in a suspension cell line (K562).

### Example 5: CRISPRi Repression of Endogenous Gene (CRISPRITEST)

Repression of genes in the CRISPRi lines were achieved by introducing lentiviral guide RNAs (Sigma Aldrich), selection with puromycin, and qRTPCR after 6 days of transduction, according to the manufacturer's protocols (see e.g., TAQMAN FAST ADVANCED CELLS-TO-CT Kit, Cat #A35374, A35377, A35378). gRNA sequences used for target gene silencing are listed in Table 1.

TABLE 1
List of gRNAs

| Target | |
|---|---|
| gene | gRNA |
| EZH2-1 | GCCCCGCTCGGCGATACCC (SEQ ID NO: 24) |
| EZH2-2 | GTCGCGTCCGACACCCGGT (SEQ ID NO: 25) |
| ATF4-1 | GACGAAGTCTATAAAGGGC (SEQ ID NO: 26) |
| ATF4-2 | CATGGCGTGAGTACCGGGG (SEQ ID NO: 27) |
| HDAC1-1 | ACCGACTGACGGTAGGGAC (SEQ ID NO: 28) |
| HDAC1-2 | GGACGGGAGGCGAGCAAGA (SEQ ID NO: 29) |
| LMNA-1 | CGGACCTCGGGATCTGGGT (SEQ ID NO: 30) |
| LMNA-2 | CCGGGCGCTGTCGGACCTC (SEQ ID NO: 31) |
| RAB1A | GCCGGCGAACCAGGAAATA (SEQ ID NO: 32) |
| NTC | AACGTGCTGACGATGCGGGC (SEQ ID NO: 33) |

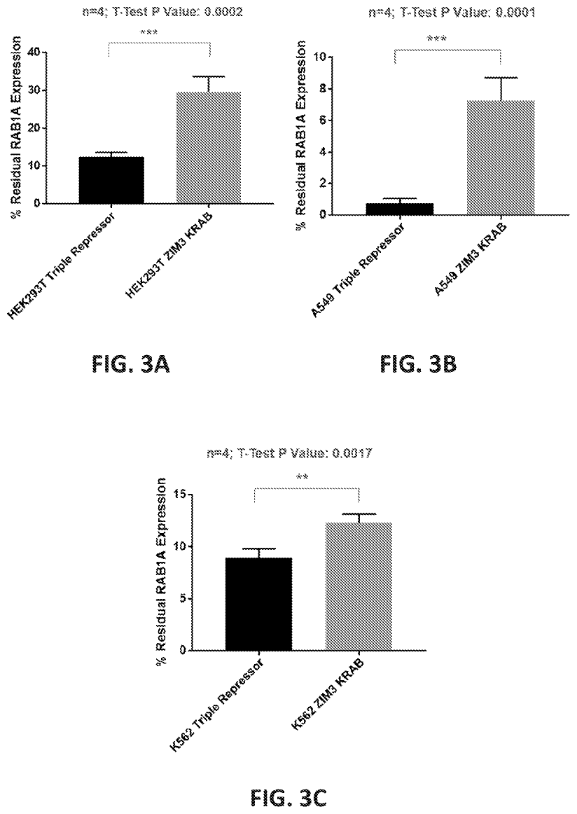

In the example shown in FIGS. 3A-3C, CRISPRi activity of dCas9 ZIM3 KRAB was compared with dCas9 Triple Repressor in HEK 293T, A549, and K562 lines by qRTPCR measurement of a targeted endogenous gene RAB1A. For the repression of an endogenous gene RAB1A, triple repressor was found to be statistically superior than ZIM3 KRAB CRISPRi system in all three cell lines (HEK293T, A549 and K562).

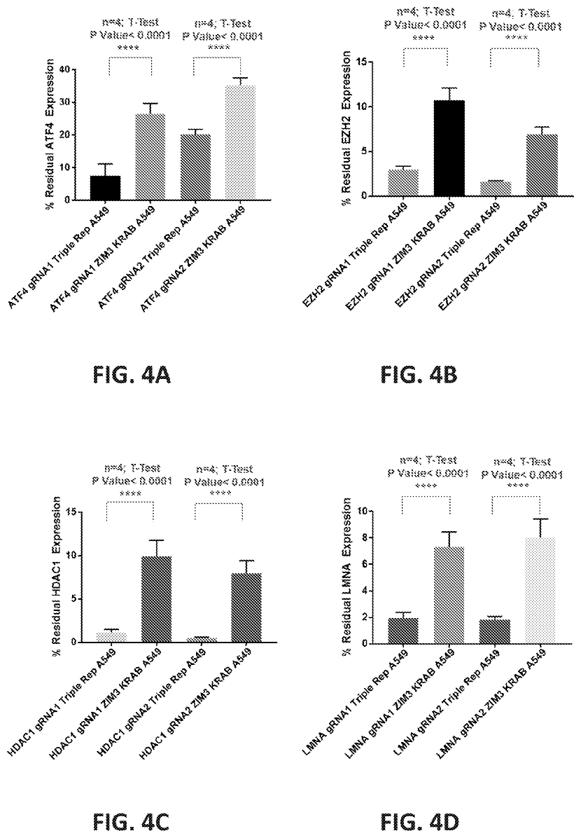

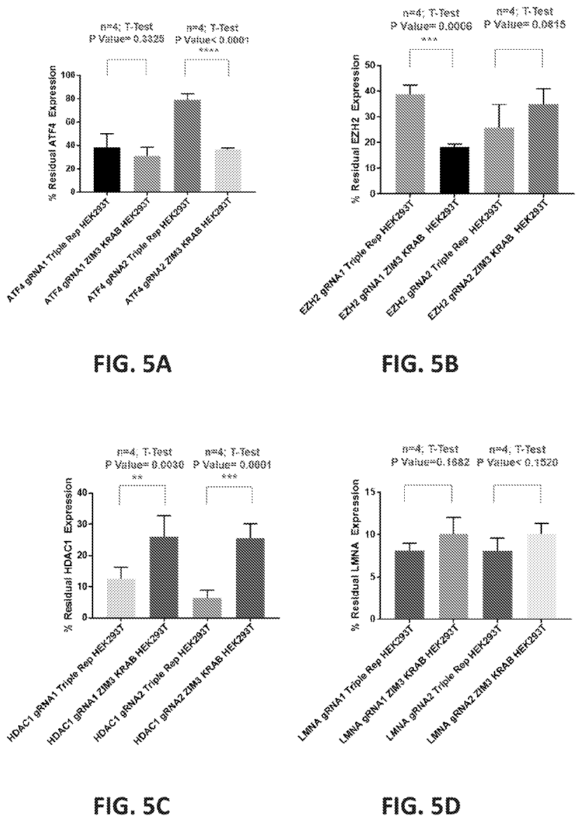

In the example shown in FIGS. 4-5, CRISPRi activity of dCas9 ZIM3 KRAB was compared with dCas9 Triple Repressor in HEK 293T and A549 lines by qRTPCR measurement of 4 targeted endogenous genes ATF4, EZH2, HDAC1, and LMNA. Each gene will be targeted with 2 gRNAs individually. As can be seen in Table 2, in 73% gene targets, the performance of triple repressor was superior to ZIM KRAB CRISPRi. In 18% gene targets, the performance of triple repressor was equivalent to ZIM3 KRAB CRISPRi. In 9% gene targets, the performance of triple repressor was inferior to ZIM3 KRAB CRISPRi. In some cases, gene repression produced by triple repressors was 1 order of magnitude higher than other KRAB-based systems. The superiority of triple repressors was observed across multiple cell lines (both adherent and suspension), multiple genes, and multiple guides. In some cases, the triple repressor performed better in A549s than HEK293Ts.

TABLE 2
Summary of Results

| | A549 | A549 | | HEK293T | K562 | K562 |
|---|---|---|---|---|---|---|
| | Triple | ZIM3 | HEK293T | ZIM3 | Triple | ZIM3 |
| | Rep | KRAB | Triple Rep | KRAB | Rep | KRAB |
| Reporter | + | − | + | − | + | − |
| RAB1A | + | − | + | − | + | − |
| ATF4 | + | − | ND | ND | + = Superior | |
| gRNA1 | | | | | − = Inferior | |
| ATF4 | + | − | − | + | ND = No Difference | |
| gRNA2 | | | | | | |
| EZH2 | + | − | − | + | | |
| gRNA1 | | | | | | |
| EZH2 | + | − | ND | ND | | |
| gRNA2 | | | | | | |
| HDAC1 | + | − | + | − | | |
| gRNA1 | | | | | | |
| HDAC1 | + | − | + | − | | |
| gRNA2 | | | | | | |
| LMNA | + | − | ND | ND | | |
| gRNA1 | | | | | | |
| LMNA | + | − | ND | ND | | |
| gRNA2 | | | | | | |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

BRIEF DECRIPTION OF SEQUENCES

SEQ ID NO: 1-Sequence of IDT #1
CTCGAGGCCACCATGAACAATTCCCAGGGAAGAGTGACCTTCGAGGATGTCAC
TGTGAACTTCACCCAGGGGGAGTGGCAGCGGCTGAATCCCGAACAGAGAAAC
TTGTACAGGGATGTGATGCTGGAGAATTACAGCAACCTTGTCTCTGTGGGACA
AGGGGAAACCACCAAACCCGATGTGATCTTGAGGTTGGAACAAGGAAAGGAG
CCATGGTTGGAGGAAGAGGAAGTGCTGGGAAGTGGCCGTGCAGAAAAAAATG
GGGACATTGGAGGGCAGATTTGGAAGCCAAAGGATGTGAAAGAGAGTCTCGG
TGGGTCTGGCGGTTCTGTGCAGGTGAAAAGGGTGCTGGAAAAATCCCCCGGCA
AACTCCTCGTGAAGATGCCCTTCCAGGCTTCCCCTGGCGGAAAAGGTGAAGGG
GGTGGCGCAACCACATCTGCCCAGGTCATGGTCATCAAGCGACCTGGAAGGAA
AAGAAAGGCCGAGGCTGACCCTCAGGCCATTCCAAAGAAACGGGGACGCAAG
CCAGGGTCCGTGGTCGCAGCTGCAGCAGCTGAGGCTAAGAAAAAGGCAGTGA
AGGAAAGCTCCATCCGCAGTGTGCAGGAGACTGTCCTGCCCATCAAGAAGAGG
AAGACTAGGGAGACCGTGTCCATCGAGGTCAAAGAAGTGGTCAAGCCCCTGCT
CGTGTCCACCCTGGGCGAAAAATCTGGAAAGGGGCTCAAAACATGCAAGTCAC
CTGGACGGAAAAGCAAGGAGTCTAGTCCAAAGGGGCGCTCAAGCTCCGCTTCT
AGTCCCCCTAAAAAGGAACACCATCACCATCACCATCACGCCGAGTCTCCT
AAGGCTCCTATGCCACTGCTCCCACCACCTCCACCACCTGAGCCACAGTCAAG
CGAAGACCCCATCAGCCCACCCGAGCCTCAGGATCTGTCCTCTAGTATTTGCA
AAGAGGAAAAGATGCCCAGAGCAGGCAGCCTGGAGAGTGATGGCTGTCCAAA
AGAACCCGCCAAGACCCAGCCTATGGTGGCAGCCGCTGCAACTACCACCACAA
CCACAACTACCACAGTGGCCGAAAAATACAAGCATCGCGGCGAGGGCGAACG
AAAGGACATTGTGTCAAGCTCCATGCCCAGACCTAACCGGGAGGAACCAGTCG
ATAGTAGGACACCCGTGACTGAGAGAGTCTCAGGCTCCGCCGGCAGCGCTGC
CGGCTCAGGGGAGTTTCCTAAG AAA AAGCGGAAAGTGCGTACG
SEQ ID NO: 2-Sequence of IDT #2
GGATCCAGTAAACGACCTGCCGCCACTAAAAAAGCCGGACAGGCTAAGAAGA
AGAAAGGAGGTTCAGGAGGATCTGGGGGGAGCGGAGGGAGCATGTCCAGGCG
GAAACAGAGCAACCCCCGGCAGATCAAGCGTTCCCTCGGAGACATGGAGGCC
AGAGAGGAGGTGCAGTTGGTGGGTGCCAGCCACATGGAGCAAAAGGCCACGG
CACCTGAAGCCCCGAGCCCTGGCTCCGGCGCAACAAACTTCTCTCTGCTGAAA
CAAGCCGGAGATGTCGAAGAGAATCCTGGACCGATGGCCAAGCCTTTGTCTCA
AGAAGAATCCACCCTCATTGAAAGAGCAACGGCTACAATCAACAGCATCCCCA
TCTCTGAAGACTACAGCGTCGCCAGCGCAGCTCTCTCTAGCGACGGCCGCATC
TTCACTGGTGTCAATGTATATCATTTTACTGGGGGACCTTGTGCAGAACTCGTG
GTGCTGGGCACTGCTGCTGCTGCGGCAGCTGGCAACCTGACTTGTATCGTCGC
GATCGGAAATGAGAACAGGGGCATCTTGAGCCCCTGCGGACGGTGCCGACAG
GTGCTTCTCGATCTGCATCCTGGGATCAAAGCCATAGTGAAGGACAGTGATGG
ACAGCCGACGGCAGTTGGGATTCGTGAATTGCTGCCCTCTGGTTATGTGTGGG
AGGGCTAAGAATTC
SEQ ID NO: 3-Sequence of IDT #3
GGATCCAGCGAGCTGATTAAGGAGAACATGCACATGAAGCTGTACATGGAGG
GCACCGTGGACAACCATCACTTCAAGTGCACATCCGAGGGCGAAGGCAAGCCC
TACGAGGGCACCCAGACCATGAGAATCAAGGTGGTCGAGGGCGGCCCTCTCCC
CTTCGCCTTCGACATCCTGGCTACTAGCTTCCTCTACGGCAGCAAGACCTTCAT
CAACCACACCCAGGGCATCCCCGACTTCTTCAAGCAGTCCTTCCCTGAGGGCTT
CACATGGGAGAGAGTCACCACATACGAAGACGGGGGCGTGCTGACCGCTACC
CAGGACACCAGCCTCCAGGACGGCTGCCTCATCTACAACGTCAAGATCAGAGG
GGTGAACTTCACATCCAACGGCCCTGTGATGCAGAAGAAAACACTCGGCTGGG
AGGCCTTCACCGAGACGCTGTACCCCGCTGACGGCGGCCTGGAAGGCAGAAAC
GACATGGCCCTGAAGCTCGTGGGCGGGAGCCATCTGATCGCAAACATCAAGAC
CACATATAGATCCAAGAAACCCGCTAAGAACCTCAAGATGCCTGGCGTCTACT
ATGTGGACTACAGACTGGAAAGAATCAAGGAGGCCAACAACGAGACCTACGT
CGAGCAGCACGAGGTGGCAGTGGCCAGATACTGCGACCTCCCTAGCAAACTGG
GGCACAAGCTTAATGGATCC
SEQ ID NO: 4-Sequence of AELIAN EFS ZIM3 KRAB dCas9 P2A Blast Lenti
Vector
AGTTTGTTTTGGCACCAAAATCAACGGGACTTTCCAAAATGTCGTAACAACTCC
GCCCCATTGACGCAAATGGGCGGTAGGCGTGTACGGTGGGAGGTCTATATAAG
CAGCGCGTTTTGCCTGTACTGGCTCTCTCTGGTTAGACCAGATCTGAGCCTGGG
AGCTCTCTGGCTAACTAGGGAACCCACTGCTTAAGCCTCAATAAAGCTTGCCTT
GAGTGCTTCAAGTAGTGTGTGCCCGTCTGTTGTGTGACTCTGGTAACTAGAGAT
CCCTCAGACCCTTTTAGTCAGTGTGGAAAATCTCTAGCAGTGGCGCCCGAACA
GGGACTTGAAAGCGAAAGGGAAACCAGAGGAGCTCTCTCGACGCAGGACTCG
GCTTGCTGAAGCGCGCACGGCAAGAGGCGAGGGGCGGCGACTGGTGAGTACG
CCAAAAATTTTGACTAGCGGAGGCTAGAAGGAGAGAGATGGGTGCGAGAGCG
TCAGTATTAAGCGGGGGAGAATTAGATCGCGATGGGAAAAAATTCGGTTAAG
GCCAGGGGGAAAGAAAAAATATAAATTAAAACATATAGTATGGGCAAGCAGG
GAGCTAGAACGATTCGCAGTTAATCCTGGCCTGTTAGAAACATCAGAAGGCTG
TAGACAAATACTGGGACAGCTACAACCATCCCTTCAGACAGGATCAGAAGAAC
TTAGATCATTATATAATACAGTAGCAACCCTCTATTGTGTGCATCAAAGGATAG
AGATAAAAGACACCAAGGAAGCTTTAGACAAGATAGAGGAAGAGCAAAACAA
AAGTAAGACCACCGCACAGCAAGCGGCCGCTGATCTTCAGACCTGGAGGAGG
AGATATGAGGGACAATTGGAGAAGTGAATTATATAAATATAAAGTAGTAAAA
ATTGAACCATTAGGAGTAGCACCCACCAAGGCAAAGAGAAGAGTGGTGCAGA
GAGAAAAAAGAGCAGTGGGAATAGGAGCTTTGTTCCTTGGGTTCTTGGGAGCA
GCAGGAAGCACTATGGGCGCAGCGTCAATGACGCTGACGGTACAGGCCAGAC
AATTATTGTCTGGTATAGTGCAGCAGCAGAACAATTTGCTGAGGGCTATTGAG
GCGCAACAGCATCTGTTGCAACTCACAGTCTGGGGCATCAAGCAGCTCCAGGC
AAGAATCCTGGCTGTGGAAAGATACCTAAAGGATCAACAGCTCCTGGGGATTT
GGGGTTGCTCTGGAAAACTCATTTGCACCACTGCTGTGCCTTGGAATGCTAGTT
GGAGTAATAAATCTCTGGAACAGATTTGGAATCACACGACCTGGATGGAGTGG
GACAGAGAAATTAACAATTACACAAGCTTAATACACTCCTTAATTGAAGAATC
GCAAAACCAGCAAGAAAAGAATGAACAAGAATTATTGGAATTAGATAAATGG
GCAAGTTTGTGGAATTGGTTTAACATAACAAATTGGCTGTGGTATATAAAATT
ATTCATAATGATAGTAGGAGGCTTGGTAGGTTTAAGAATAGTTTTTGCTGTACT
TTCTATAGTGAATAGAGTTAGGCAGGGATATTCACCATTATCGTTTCAGACCCA
CCTCCCAACCCCGAGGGGACCCGACAGGCCCGAAGGAATAGAAGAAGAAGGT
GGAGAGAGAGACAGAGACAGATCCATTCGATTAGTGAACGGATCGGCACTGC
GTGCGCCAATTCTGCAGACAAATGGCAGTATTCATCCACAATTTTAAAAGAAA
AGGGGGGATTGGGGGGTACAGTGCAGGGGAAAGAATAGTAGACATAATAGCA
ACAGACATACAAACTAAAGAATTACAAAAACAAATTACAAAAATTCAAAATTT
TCGGGTTTATTACAGGGACAGCAGAGATCCAGTTTGGTTAATTAGGGTTAATT
AGCTAGCGGCTCCGGTGCCCGTCAGTGGGCAGAGCGCACATCGCCCACAGTCC
CCGAGAAGTTGGGGGGAGGGGTCGGCAATTGATCCGGTGCCTAGAGAAGGTG
GCGCGGGGTAAACTGGGAAAGTGATGTCGTGTACTGGCTCCGCCTTTTTCCCG
AGGGTGGGGGAGAACCGTATATAAGTGCAGTAGTCGCCGTGAACGTTCTTTTT
CGCAACGGGTTTGCCGCCAGAACACAGGCTCGAGGCCACCATGAACAATTCCC
AGGGAAGAGTGACCTTCGAGGATGTCACTGTGAACTTCACCCAGGGGGAGTGG
CAGCGGCTGAATCCCGAACAGAGAAACTTGTACAGGGATGTGATGCTGGAGA
ATTACAGCAACCTTGTCTCTGTGGGACAAGGGGAAACCACCAAACCCGATGTG
ATCTTGAGGTTGGAACAAGGAAAGGAGCCATGGTTGGAGGAAGAGGAAGTGC
TGGGAAGTGGCCGTGCAGAAAAAAATGGGGACATTGGAGGGCAGATTTGGAA
GCCAAAGGATGTGAAAGAGAGTCTCCGTACGGACAAGAAGTACAGCATCGGC
CTGGCCATCGGCACCAACTCTGTGGGCTGGGCCGTGATCACCGACGAGTACAA
GGTGCCCAGCAAGAAATTCAAGGTGCTGGGCAACACCGACCGGCACAGCATC
AAGAAGAACCTGATCGGCGCCCTGCTGTTCGACAGCGGAGAAACAGCCGAGG
CCACCCGGCTGAAGAGAACCGCCAGAAGAAGATACACCAGACGGAAGAACCG
GATCTGCTATCTGCAAGAGATCTTCAGCAACGAGATGGCCAAGGTGGACGACA
GCTTCTTCCACAGACTGGAAGAGTCCTTCCTGGTGGAAGAGGATAAGAAGCAC
GAGCGGCACCCCATCTTCGGCAACATCGTGGACGAGGTGGCCTACCACGAGAA
GTACCCCACCATCTACCACCTGAGAAAGAAACTGGTGGACAGCACCGACAAG
GCCGACCTGCGGCTGATCTATCTGGCCCTGGCCCACATGATCAAGTTCCGGGG
CCACTTCCTGATCGAGGGCGACCTGAACCCCGACAACAGCGACGTGGACAAGC
TGTTCATCCAGCTGGTGCAGACCTACAACCAGCTGTTCGAGGAAAACCCCATC
AACGCCAGCGGCGTGGACGCCAAGGCCATCCTGTCTGCCAGACTGAGCAAGA
GCAGACGGCTGGAAAATCTGATCGCCCAGCTGCCCGGCGAGAAGAAGAATGG
CCTGTTCGGCAACCTGATTGCCCTGAGCCTGGGCCTGACCCCCAACTTCAAGA
GCAACTTCGACCTGGCCGAGGATGCCAAACTGCAGCTGAGCAAGGACACCTAC
GACGACGACCTGGACAACCTGCTGGCCCAGATCGGCGACCAGTACGCCGACCT
GTTTCTGGCCGCCAAGAACCTGTCCGACGCCATCCTGCTGAGCGACATCCTGA
GAGTGAACACCGAGATCACCAAGGCCCCCCTGAGCGCCTCTATGATCAAGAGA
TACGACGAGCACCACCAGGACCTGACCCTGCTGAAAGCTCTCGTGCGGCAGCA
GCTGCCTGAGAAGTACAAAGAGATTTTCTTCGACCAGAGCAAGAACGGCTACG
CCGGCTACATCGATGGCGGAGCCAGCCAGGAAGAGTTCTACAAGTTCATCAAG
CCCATCCTGGAAAAGATGGACGGCACCGAGGAACTGCTCGTGAAGCTGAACA
GAGAGGACCTGCTGCGGAAGCAGCGGACCTTCGACAACGGCAGCATCCCCCA
CCAGATCCACCTGGGAGAGCTGCACGCCATTCTGCGGCGGCAGGAAGATTTTT
ACCCATTCCTGAAGGACAACCGGGAAAAGATCGAGAAGATCCTGACCTTCCGC
ATCCCCTACTACGTGGGCCCTCTGGCCAGGGGAAACAGCAGATTCGCCTGGAT
GACCAGAAAGAGCGAGGAAACCATCACCCCCTGGAACTTCGAGGAAGTGGTG
GACAAGGGCGCCAGCGCCCAGAGCTTCATCGAGCGGATGACCAACTTCGATAA
GAACCTGCCCAACGAGAAGGTGCTGCCCAAGCACAGCCTGCTGTACGAGTACT
TCACCGTGTACAACGAGCTGACCAAAGTGAAATACGTGACCGAGGGAATGAG
AAAGCCCGCCTTCCTGAGCGGCGAGCAGAAAAAAGCCATCGTGGACCTGCTGT
TCAAGACCAACCGGAAAGTGACCGTGAAGCAGCTGAAAGAGGACTACTTCAA
GAAAATCGAGTGCTTCGACTCCGTGGAAATCTCCGGCGTGGAAGATCGGTTCA
ACGCCTCCCTGGGCACATACCACGATCTGCTGAAAATTATCAAGGACAAGGAC
TTCCTGGACAATGAGGAAAACGAGGACATTCTGGAAGATATCGTGCTGACCCT
GACACTGTTTGAGGACAGAGAGATGATCGAGGAACGGCTGAAAACCTATGCC
CACCTGTTCGACGACAAAGTGATGAAGCAGCTGAAGCGGCGGAGATACACCG
GCTGGGGCAGGCTGAGCCGGAAGCTGATCAACGGCATCCGGGACAAGCAGTC
CGGCAAGACAATCCTGGATTTCCTGAAGTCCGACGGCTTCGCCAACAGAAACT
TCATGCAGCTGATCCACGACGACAGCCTGACCTTTAAAGAGGACATCCAGAAA
GCCCAGGTGTCCGGCCAGGGCGATAGCCTGCACGAGCACATTGCCAATCTGGC
CGGCAGCCCCGCCATTAAGAAGGGCATCCTGCAGACAGTGAAGGTGGTGGAC
GAGCTCGTGAAAGTGATGGGCCGGCACAAGCCCGAGAACATCGTGATCGAAA
TGGCCAGAGAGAACCAGACCACCCAGAAGGGACAGAAGAACAGCCGCGAGA
GAATGAAGCGGATCGAAGAGGGCATCAAAGAGCTGGGCAGCCAGATCCTGAA
AGAACACCCCGTGGAAAACACCCAGCTGCAGAACGAGAAGCTGTACCTGTACT
ACCTGCAGAATGGGCGGGATATGTACGTGGACCAGGAACTGGACATCAACCG
GCTGTCCGACTACGATGTGGACGCTATCGTGCCTCAGAGCTTTCTGAAGGACG
ACTCCATCGATAACAAAGTGCTGACTCGGAGCGACAAGAACCGGGGCAAGAG
CGACAACGTGCCCTCCGAAGAGGTCGTGAAGAAGATGAAGAACTACTGGCGC
CAGCTGCTGAATGCCAAGCTGATTACCCAGAGGAAGTTCGACAATCTGACCAA
GGCCGAGAGAGGCGGCCTGAGCGAACTGGATAAGGCCGGCTTCATCAAGAGA
CAGCTGGTGGAAACCCGGCAGATCACAAAGCACGTGGCACAGATCCTGGACTC
CCGGATGAACACTAAGTACGACGAGAACGACAAACTGATCCGGGAAGTGAAA
GTGATCACCCTGAAGTCCAAGCTGGTGTCCGATTTCCGGAAGGATTTCCAGTTT
TACAAAGTGCGCGAGATCAACAACTACCACCACGCCCACGACGCCTACCTGAA
CGCCGTCGTGGGAACCGCCCTGATCAAAAAGTACCCTAAGCTGGAAAGCGAGT
TCGTGTACGGCGACTACAAGGTGTACGACGTGCGGAAGATGATCGCCAAGAGC
GAGCAGGAAATCGGCAAGGCTACCGCCAAGTACTTCTTCTACAGCAACATCAT
GAACTTTTTCAAGACCGAGATTACCCTGGCCAACGGCGAGATCCGGAAGCGGC
CTCTGATCGAGACAAACGGCGAAACAGGCGAGATCGTGTGGGATAAGGGCCG
GGACTTTGCCACCGTGCGGAAAGTGCTGTCTATGCCCCAAGTGAATATCGTGA
AAAAGACCGAGGTGCAGACAGGCGGCTTCAGCAAAGAGTCTATCCTGCCCAA
GAGGAACAGCGACAAGCTGATCGCCAGAAAGAAGGACTGGGACCCTAAGAAG
TACGGCGGCTTCGACAGCCCCACCGTGGCCTATTCTGTGCTGGTGGTGGCCAA
AGTGGAAAAGGGCAAGTCCAAGAAACTGAAGAGTGTGAAAGAGCTGCTGGGG
ATCACCATCATGGAAAGAAGCAGCTTCGAGAAGAATCCCATCGACTTTCTGGA
AGCCAAGGGCTACAAAGAAGTGAAAAAGGACCTGATCATCAAGCTGCCTAAG
TACTCCCTGTTCGAGCTGGAAAACGGCCGGAAGAGAATGCTGGCCTCTGCCGG
CGAACTGCAGAAGGGAAACGAACTGGCCCTGCCCTCCAAATATGTGAACTTCC
TGTACCTGGCCAGCCACTATGAGAAGCTGAAGGGCTCCCCCGAGGATAATGAG
CAGAAACAGCTGTTTGTGGAACAGCACAAACACTACCTGGACGAGATCATCGA
GCAGATCAGCGAGTTCTCCAAGAGAGTGATCCTGGCCGACGCTAATCTGGACA
AGGTGCTGAGCGCCTACAACAAGCACAGAGACAAGCCTATCAGAGAGCAGGC
CGAGAATATCATCCACCTGTTTACCCTGACCAATCTGGGAGCCCCTGCCGCCTT
CAAGTACTTTGACACCACCATCGACCGGAAGAGGTACACCAGCACCAAAGAG
GTGCTGGACGCCACCCTGATCCACCAGAGCATCACCGGCCTGTACGAGACACG
GATCGACCTGTCTCAGCTGGGAGGCGACGCCTATCCCTATGACGTGCCCGATT
ATGCCAGCCTGGGCAGCGGCTCCCCCAAGAAAAAACGCAAGGTGGAAGGATC
CGGCGCAACAAACTTCTCTCTGCTGAAACAAGCCGGAGATGTCGAAGAGAATC
CTGGACCGATGGCCAAGCCTTTGTCTCAAGAAGAATCCACCCTCATTGAAAGA
GCAACGGCTACAATCAACAGCATCCCCATCTCTGAAGACTACAGCGTCGCCAG
CGCAGCTCTCTCTAGCGACGGCCGCATCTTCACTGGTGTCAATGTATATCATTT
TACTGGGGGACCTTGTGCAGAACTCGTGGTGCTGGGCACTGCTGCTGCTGCGG
CAGCTGGCAACCTGACTTGTATCGTCGCGATCGGAAATGAGAACAGGGGCATC
TTGAGCCCCTGCGGACGGTGCCGACAGGTGCTTCTCGATCTGCATCCTGGGATC
AAAGCCATAGTGAAGGACAGTGATGGACAGCCGACGGCAGTTGGGATTCGTG
AATTGCTGCCCTCTGGTTATGTGTGGGAGGGCTAAGAATTCAATCAACCTCTGG
ATTACAAAATTTGTGAAAGATTGACTGGTATTCTTAACTATGTTGCTCCTTTTA
CGCTATGTGGATACGCTGCTTTAATGCCTTTGTATCATGCTATTGCTTCCCGTAT
GGCTTTCATTTTCTCCTCCTTGTATAAATCCTGGTTGCTGTCTCTTTATGAGGAG
TTGTGGCCCGTTGTCAGGCAACGTGGCGTGGTGTGCACTGTGTTTGCTGACGCA
ACCCCCACTGGTTGGGGCATTGCCACCACCTGTCAGCTCCTTTCCGGGACTTTC
GCTTTCCCCCTCCCTATTGCCACGGCGGAACTCATCGCCGCCTGCCTTGCCCGC
TGCTGGACAGGGGCTCGGCTGTTGGGCACTGACAATTCCGTGGTGTTGTCGGG
GAAGCTGACGTCCTTTCCATGGCTGCTCGCCTGTGTTGCCACCTGGATTCTGCG
CGGGACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCCCTCA
GACGAGTCGGATCTCCCTTTGGGCCGCCTCCCCGCGAGACGTTTCATTTCCGTC
TCTGGTACCACTTTTTAAAAGAAAAGGGGGGACTGGAAGGGCTAATTCACTCC
CAACGAAGTCAAGATATCCTTGATCTGTGGATCGTTAACTACCACACACAAGG
CTACTTCCCTGATTGGCAGAACTACACACCAGGGCCAGGGATCAAATATCCAC
TGACCTTTGGATGGTGCTACAAGCTAGTACCAGTTGAGCAAGAGAAGGTAGAA
GAAGCCAATGAAGGAGAGAACACCCGCTTGTTACACCCTGTGAGCCTGCATGG
GATGGATGACCCGGAGAGAGAAGTATTAGAGTGGAGGGTTAACTTAATTAAG
ACAGCCGCCTAGCATTTCATCACATGGCCCGAGAGCTGCATCCGGACTGTACT
GGCTCTCTCTGGTTAGACCAGATCTGAGCCTGGGAGCTCTCTGGCTAACTAGG
GAACCCACTGCTTAAGCCTCAATAAAGCTTGCCTTGAGTGCTTCAAGTAGTGTG
TGCCCGTCTGTTGTGTGACTCTGGTAACTAGAGATCCCTCAGACCCTTTTAGTC
AGTGTGGAAAATCTCTAGCAGGGCCCTCTAGAGTTTAAACCCGCTGATCAGCC
TCGACTGTGCCTTCTAGTTGCCAGCCATCTGTTGTTTGCCCCTCCCCCGTGCCTT
CCTTGACCCTGGAAGGTGCCACTCCCACTGTCCTTTCCTAATAAAATGAGGAA
ATTGCATCGCATTGTCTGAGTAGGTGTCATTCTATTCTGGGGGGTGGGGTGGGG
CAGGACAGCAAGGGGGAGGATTGGGAAGAGAATAGCAGGCATGCTGGGGATG
CGGTGGGCTCTATGGCTTCTGAGGCGGAAAGAACCAGCTGGGGCTCTAGGGGG
TATCCCCACGCGCCCTGTAGCGGCGCATTAAGCGCGGCGGGTGTGGTGGTTAC
GCGCAGCGTGACCGCTACACTTGCCAGCGCCCTAGCGCCCGCTCCTTTCGCTTT
CTTCCCTTCCTTTCTCGCCACGTTCGCCGGCTTTCCCCGTCAAGCTCTAAATCGG
GGGCTCCCTTTAGGGTTCCGATTTAGTGCTTTACGGCACCTCGACCCCAAAAAA
CTTGATTAGGGTGATGGTTCACGTAGTGGGCCATCGCCCTGATAGACGGTTTTT
CGCCCTTTGACGTTGGAGTCCACGTTCTTTAATAGTGGACTCTTGTTCCAAACT
GGAACAACACTCAACCCTATCTCGGTCTATTCTTTTGATTTATAAGGGATTTTG
CCGATTTCGGCCTATTGGTTAAAAAATGAGCTGATTTAACAAAAATTTAACGC
GAATTAATTCTGTGGAATGTGTGTCAGTTAGGGTGTGGAAAGTCCCCAGGCTC
CCCAGCAGGCAGAAGTATGCAAAGCATGCATCTCAATTAGTCAGCAACCAGGT
GTGGAAAGTCCCCAGGCTCCCCAGCAGGCAGAAGTATGCAAAGCATGCATCTC
AATTAGTCAGCAACCATAGTCCCGCCCCTAACTCCGCCCATCCCGCCCCTAACT
CCGCCCAGTTCCGCCCATTCTCCGCCCCATGGCTGACTAATTTTTTTTATTTATG
CAGAGGCCGAGGCCGCCTCTGCCTCTGAGCTATTCCAGAAGTAGTGAGGAGGC
TTTTTTGGAGGCCTAGGCTTTTGCAAAAAGCTCCCGGGAGCTTGTATATCCATT
TTCGGATCTGATCAGCACGTGTTGACAATTAATCATCGGCATAGTATATCGGCA
TAGTATAATACGACAAGGTGAGGAACTAAACCATGGCCAAGTTGACCAGTGCC
GTTCCGGTGCTCACCGCGCGCGACGTCGCCGGAGCGGTCGAGTTCTGGACCGA
CCGGCTCGGGTTCTCCCGGGACTTCGTGGAGGACGACTTCGCCGGTGTGGTCC
GGGACGACGTGACCCTGTTCATCAGCGCGGTCCAGGACCAGGTGGTGCCGGAC
AACACCCTGGCCTGGGTGTGGGTGCGCGGCCTGGACGAGCTGTACGCCGAGTG
GTCGGAGGTCGTGTCCACGAACTTCCGGGACGCCTCCGGGCCGGCCATGACCG
AGATCGGCGAGCAGCCGTGGGGGGGGAGTTCGCCCTGCGCGACCCGGCCGG
CAACTGCGTGCACTTCGTGGCCGAGGAGCAGGACTGACACGTGCTACGAGATT
TCGATTCCACCGCCGCCTTCTATGAAAGGTTGGGCTTCGGAATCGTTTTCCGGG
ACGCCGGCTGGATGATCCTCCAGCGCGGGGATCTCATGCTGGAGTTCTTCGCC
CACCCCAACTTGTTTATTGCAGCTTATAATGGTTACAAATAAAGCAATAGCATC
ACAAATTTCACAAATAAAGCATTTTTTTCACTGCATTCTAGTTGTGGTTTGTCC
AAACTCATCAATGTATCTTATCATGTCTGTATACCGTCGACCTCTAGCTAGAGC
TTGGCGTAATCATGGTCATAGCTGTTTCCTGTGTGAAATTGTTATCCGCTCACA
ATTCCACACAACATACGAGCCGGAAGCATAAAGTGTAAAGCCTGGGGTGCCTA
ATGAGTGAGCTAACTCACATTAATTGCGTTGCGCTCACTGCCCGCTTTCCAGTC
GGGAAACCTGTCGTGCCAGCTGCATTAATGAATCGGCCAACGCGCGGGGAGA
GGCGGTTTGCGTATTGGGCGCTCTTCCGCTTCCTCGCTCACTGACTCGCTGCGC
TCGGTCGTTCGGCTGCGGCGAGCGGTATCAGCTCACTCAAAGGCGGTAATACG
GTTATCCACAGAATCAGGGGATAACGCAGGAAAGAACATGTGAGCAAAAGGC
CAGCAAAAGGCCAGGAACCGTAAAAAGGCCGCGTTGCTGGCGTTTTTCCATAG
GCTCCGCCCCCCTGACGAGCATCACAAAAATCGACGCTCAAGTCAGAGGTGGC
GAAACCCGACAGGACTATAAAGATACCAGGCGTTTCCCCCTGGAAGCTCCCTC
GTGCGCTCTCCTGTTCCGACCCTGCCGCTTACCGGATACCTGTCCGCCTTTCTCC
CTTCGGGAAGCGTGGCGCTTTCTCATAGCTCACGCTGTAGGTATCTCAGTTCGG
TGTAGGTCGTTCGCTCCAAGCTGGGCTGTGTGCACGAACCCCCCGTTCAGCCCG
ACCGCTGCGCCTTATCCGGTAACTATCGTCTTGAGTCCAACCCGGTAAGACAC
GACTTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACACTA
GAAGAACAGTATTTGGTATCTGCGCTCTGCTGAAGCCAGTTACCTTCGGAAAA
AGAGTTGGTAGCTCTTGATCCGGCAAACAAACCACCGCTGGTAGCGGTGGTTT
TTTTGTTTGCAAGCAGCAGATTACGCGCAGAAAAAAAGGATCTCAAGAAGATC
CTTTGATCTTTTCTACGGGGTCTGACGCTCAGTGGAACGAAAACTCACGTTAAG
GGATTTTGGTCATGAGATTATCAAAAAGGATCTTCACCTAGATCCTTTTAAATT
AAAAATGAAGTTTTAAATCAATCTAAAGTATATATGAGTAAACTTGGTCTGAC
AGTTACCAATGCTTAATCAGTGAGGCACCTATCTCAGCGATCTGTCTATTTCGT
TCATCCATAGTTGCCTGACTCCCCGTCGTGTAGATAACTACGATACGGGAGGG
CTTACCATCTGGCCCCAGTGCTGCAATGATACCGCGGGACCCACGCTCACCGG
CTCCAGATTTATCAGCAATAAACCAGCCAGCCGGAAGGGCCGAGCGCAGAAG
TGGTCCTGCAACTTTATCCGCCTCCATCCAGTCTATTAATTGTTGCCGGGAAGC
TAGAGTAAGTAGTTCGCCAGTTAATAGTTTGCGCAACGTTGTTGCCATTGCTAC
AGGCATCGTGGTGTCACGCTCGTCGTTTGGTATGGCTTCATTCAGCTCCGGTTC
CCAACGATCAAGGCGAGTTACATGATCCCCCATGTTGTGCAAAAAAGCGGTTA
GCTCCTTCGGTCCTCCGATCGTTGTCAGAAGTAAGTTGGCCGCAGTGTTATCAC
TCATGGTTATGGCAGCACTGCATAATTCTCTTACTGTCATGCCATCCGTAAGAT
GCTTTTCTGTGACTGGTGAGTACTCAACCAAGTCATTCTGAGAATAGTGTATGC
GGCGACCGAGTTGCTCTTGCCCGGCGTCAATACGGGATAATACCGCGCCACAT
AGCAGAACTTTAAAAGTGCTCATCATTGGAAAACGTTCTTCGGGGCGAAAACT
CTCAAGGATCTTACCGCTGTTGAGATCCAGTTCGATGTAACCCACTCGTGCACC
CAACTGATCTTCAGCATCTTTTACTTTCACCAGCGTTTCTGGGTGAGCAAAAAC
AGGAAGGCAAAATGCCGCAAAAAAGGGAATAAGGGCGACACGGAAATGTTGA
ATACTCATACTCTTCCTTTTTCAATATTATTGAAGCATTTATCAGGGTTATTGTC
TCATGAGCGGATACATATTTGAATGTATTTAGAAAAATAAACAAATAGGGGTT
CCGCGCACATTTCCCCGAAAAGTGCCACCTGACGTCGACGGATCGGGAGATCT
CCCGATCCCCTATGGTGCACTCTCAGTACAATCTGCTCTGATGCCGCATAGTTA
AGCCAGTATCTGCTCCCTGCTTGTGTGTTGGAGGTCGCTGAGTAGTGCGCGAGC
AAAATTTAAGCTACAACAAGGCAAGGCTTGACCGACAATTGCATGAAGAATCT
GCTTAGGGTTAGGCGTTTTGCGCTGCTTCGCGATGTACGGGCCAGATATACGC
GTTGACATTGATTATTGACTAGTTATTAATAGTAATCAATTACGGGGTCATTAG
TTCATAGCCCATATATGGAGTTCCGCGTTACATAACTTACGGTAAATGGCCCGC
CTGGCTGACCGCCCAACGACCCCCGCCCATTGACGTCAATAATGACGTATGTT
CCCATAGTAACGCCAATAGGGACTTTCCATTGACGTCAATGGGTGGAGTATTT
ACGGTAAACTGCCCACTTGGCAGTACATCAAGTGTATCATATGCCAAGTACGC
CCCCTATTGACGTCAATGACGGTAAATGGCCCGCCTGGCATTATGCCCAGTAC
ATGACCTTATGGGACTTTCCTACTTGGCAGTACATCTACGTATTAGTCATCGCT
ATTACCATGGTGATGCGGTTTTGGCAGTACATCAATGGGCGTGGATAGCGGTT
TGACTCACGGGGATTTCCAAGTCTCCACCCCATTGACGTCAATGGG
SEQ ID NO: 5-DNA Sequence of the IDT fragment #1 in lentiviral vector
TCGAGGCCACCATGAACAATTCCCAGGGAAGAGTGACCTTCGAGGATGTCACT
GTGAACTTCACCCAGGGGGAGTGGCAGCGGCTGAATCCCGAACAGAGAAACT
TGTACAGGGATGTGATGCTGGAGAATTACAGCAACCTTGTCTCTGTGGGACAA
GGGGAAACCACCAAACCCGATGTGATCTTGAGGTTGGAACAAGGAAAGGAGC
CATGGTTGGAGGAAGAGGAAGTGCTGGGAAGTGGCCGTGCAGAAAAAAATGG
GGACATTGGAGGGCAGATTTGGAAGCCAAAGGATGTGAAAGAGAGTCTCGGT
GGGTCTGGCGGTTCTGTGCAGGTGAAAAGGGTGCTGGAAAAATCCCCCGGCAA
ACTCCTCGTGAAGATGCCCTTCCAGGCTTCCCCTGGCGGAAAAGGTGAAGGGG
GTGGCGCAACCACATCTGCCCAGGTCATGGTCATCAAGCGACCTGGAAGGAAA
AGAAAGGCCGAGGCTGACCCTCAGGCCATTCCAAAGAAACGGGGACGCAAGC
CAGGGTCCGTGGTCGCAGCTGCAGCAGCTGAGGCTAAGAAAAAGGCAGTGAA
GGAAAGCTCCATCCGCAGTGTGCAGGAGACTGTCCTGCCCATCAAGAAGAGGA
AGACTAGGGAGACCGTGTCCATCGAGGTCAAAGAAGTGGTCAAGCCCCTGCTC
GTGTCCACCCTGGGCGAAAAATCTGGAAAGGGGCTCAAAACATGCAAGTCACC
TGGACGGAAAAGCAAGGAGTCTAGTCCAAAGGGGCGCTCAAGCTCCGCTTCTA
GTCCCCCTAAAAAGGAACACCATCACCATCACCATCACGCCGAGTCTCCTAAG
GCTCCTATGCCACTGCTCCCACCACCTCCACCACCTGAGCCACAGTCAAGCGA
AGACCCCATCAGCCCACCCGAGCCTCAGGATCTGTCCTCTAGTATTTGCAAAG
AGGAAAAGATGCCCAGAGCAGGCAGCCTGGAGAGTGATGGCTGTCCAAAAGA
ACCCGCCAAGACCCAGCCTATGGTGGCAGCCGCTGCAACTACCACCACAACCA
CAACTACCACAGTGGCCGAAAAATACAAGCATCGCGGCGAGGGCGAACGAAA
GGACATTGTGTCAAGCTCCATGCCCAGACCTAACCGGGAGGAACCAGTCGATA
GTAGGACACCCGTGACTGAGAGAGTCTCAGGCTCCGCCGGCAGCGCTGCCGGC
TCAGGGGAGTTTCCTAAGAAAAAGCGGAAAGTGCGTACGGACAAGAAGTACA
GCATCGGCCTGGCCATCGGCACCAACTCTGTGGGCTGGGCCGTGATCACCGAC
GAGTACAAGGTGCCCAGCAAGAAATTCAAGGTGCTGGGCAACACCGACCGGC
ACAGCATCAAGAAGAACCTGATCGGCGCCCTGCTGTTCGACAGCGGAGAAAC
AGCCGAGGCCACCCGGCTGAAGAGAACCGCCAGAAGAAGATACACCAGACGG
AAGAACCGGATCTGCTATCTGCAAGAGATCTTCAGCAACGAGATGGCCAAGGT
GGACGACAGCTTCTTCCACAGACTGGAAGAGTCCTTCCTGGTGGAAGAGGATA
AGAAGCACGAGCGGCACCCCATCTTCGGCAACATCGTGGACGAGGTGGCCTAC
CACGAGAAGTACCCCACCATCTACCACCTGAGAAAGAAACTGGTGGACAGCA
CCGACAAGGCCGACCTGCGGCTGATCTATCTGGCCCTGGCCCACATGATCAAG
TTCCGGGGCCACTTCCTGATCGAGGGCGACCTGAACCCCGACAACAGCGACGT
GGACAAGCTGTTCATCCAGCTGGTGCAGACCTACAACCAGCTGTTCGAGGAAA
ACCCCATCAACGCCAGCGGCGTGGACGCCAAGGCCATCCTGTCTGCCAGACTG
AGCAAGAGCAGACGGCTGGAAAATCTGATCGCCCAGCTGCCCGGCGAGAAGA
AGAATGGCCTGTTCGGCAACCTGATTGCCCTGAGCCTGGGCCTGACCCCCAAC
TTCAAGAGCAACTTCGACCTGGCCGAGGATGCCAAACTGCAGCTGAGCAAGGA
CACCTACGACGACGACCTGGACAACCTGCTGGCCCAGATCGGCGACCAGTACG
CCGACCTGTTTCTGGCCGCCAAGAACCTGTCCGACGCCATCCTGCTGAGCGAC
ATCCTGAGAGTGAACACCGAGATCACCAAGGCCCCCCTGAGCGCCTCTATGAT
CAAGAGATACGACGAGCACCACCAGGACCTGACCCTGCTGAAAGCTCTCGTGC
GGCAGCAGCTGCCTGAGAAGTACAAAGAGATTTTCTTCGACCAGAGCAAGAAC
GGCTACGCCGGCTACATCGATGGCGGAGCCAGCCAGGAAGAGTTCTACAAGTT
CATCAAGCCCATCCTGGAAAAGATGGACGGCACCGAGGAACTGCTCGTGAAG
CTGAACAGAGAGGACCTGCTGCGGAAGCAGCGGACCTTCGACAACGGCAGCA
TCCCCCACCAGATCCACCTGGGAGAGCTGCACGCCATTCTGCGGCGGCAGGAA
GATTTTTACCCATTCCTGAAGGACAACCGGGAAAAGATCGAGAAGATCCTGAC
CTTCCGCATCCCCTACTACGTGGGCCCTCTGGCCAGGGGAAACAGCAGATTCG
CCTGGATGACCAGAAAGAGCGAGGAAACCATCACCCCCTGGAACTTCGAGGA
AGTGGTGGACAAGGGCGCCAGCGCCCAGAGCTTCATCGAGCGGATGACCAAC
TTCGATAAGAACCTGCCCAACGAGAAGGTGCTGCCCAAGCACAGCCTGCTGTA
CGAGTACTTCACCGTGTACAACGAGCTGACCAAAGTGAAATACGTGACCGAGG
GAATGAGAAAGCCCGCCTTCCTGAGCGGCGAGCAGAAAAAAGCCATCGTGGA
CCTGCTGTTCAAGACCAACCGGAAAGTGACCGTGAAGCAGCTGAAAGAGGAC
TACTTCAAGAAAATCGAGTGCTTCGACTCCGTGGAAATCTCCGGCGTGGAAGA
TCGGTTCAACGCCTCCCTGGGCACATACCACGATCTGCTGAAAATTATCAAGG
ACAAGGACTTCCTGGACAATGAGGAAAACGAGGACATTCTGGAAGATATCGT
GCTGACCCTGACACTGTTTGAGGACAGAGAGATGATCGAGGAACGGCTGAAA
ACCTATGCCCACCTGTTCGACGACAAAGTGATGAAGCAGCTGAAGCGGCGGAG
ATACACCGGCTGGGGCAGGCTGAGCCGGAAGCTGATCAACGGCATCCGGGAC
AAGCAGTCCGGCAAGACAATCCTGGATTTCCTGAAGTCCGACGGCTTCGCCAA
CAGAAACTTCATGCAGCTGATCCACGACGACAGCCTGACCTTTAAAGAGGACA
TCCAGAAAGCCCAGGTGTCCGGCCAGGGCGATAGCCTGCACGAGCACATTGCC
AATCTGGCCGGCAGCCCCGCCATTAAGAAGGGCATCCTGCAGACAGTGAAGGT
GGTGGACGAGCTCGTGAAAGTGATGGGCCGGCACAAGCCCGAGAACATCGTG
ATCGAAATGGCCAGAGAGAACCAGACCACCCAGAAGGGACAGAAGAACAGCC
GCGAGAGAATGAAGCGGATCGAAGAGGGCATCAAAGAGCTGGGCAGCCAGAT
CCTGAAAGAACACCCCGTGGAAAACACCCAGCTGCAGAACGAGAAGCTGTAC
CTGTACTACCTGCAGAATGGGCGGGATATGTACGTGGACCAGGAACTGGACAT
CAACCGGCTGTCCGACTACGATGTGGACGCTATCGTGCCTCAGAGCTTTCTGA
AGGACGACTCCATCGATAACAAAGTGCTGACTCGGAGCGACAAGAACCGGGG
CAAGAGCGACAACGTGCCCTCCGAAGAGGTCGTGAAGAAGATGAAGAACTAC
TGGCGCCAGCTGCTGAATGCCAAGCTGATTACCCAGAGGAAGTTCGACAATCT
GACCAAGGCCGAGAGAGGCGGCCTGAGCGAACTGGATAAGGCCGGCTTCATC
AAGAGACAGCTGGTGGAAACCCGGCAGATCACAAAGCACGTGGCACAGATCC
TGGACTCCCGGATGAACACTAAGTACGACGAGAACGACAAACTGATCCGGGA
AGTGAAAGTGATCACCCTGAAGTCCAAGCTGGTGTCCGATTTCCGGAAGGATT
TCCAGTTTTACAAAGTGCGCGAGATCAACAACTACCACCACGCCCACGACGCC
TACCTGAACGCCGTCGTGGGAACCGCCCTGATCAAAAAGTACCCTAAGCTGGA
AAGCGAGTTCGTGTACGGCGACTACAAGGTGTACGACGTGCGGAAGATGATCG
CCAAGAGCGAGCAGGAAATCGGCAAGGCTACCGCCAAGTACTTCTTCTACAGC
AACATCATGAACTTTTTCAAGACCGAGATTACCCTGGCCAACGGCGAGATCCG
GAAGCGGCCTCTGATCGAGACAAACGGCGAAACAGGCGAGATCGTGTGGGAT
AAGGGCCGGGACTTTGCCACCGTGCGGAAAGTGCTGTCTATGCCCCAAGTGAA
TATCGTGAAAAAGACCGAGGTGCAGACAGGCGGCTTCAGCAAAGAGTCTATCC
TGCCCAAGAGGAACAGCGACAAGCTGATCGCCAGAAAGAAGGACTGGGACCC
TAAGAAGTACGGCGGCTTCGACAGCCCCACCGTGGCCTATTCTGTGCTGGTGG
TGGCCAAAGTGGAAAAGGGCAAGTCCAAGAAACTGAAGAGTGTGAAAGAGCT
GCTGGGGATCACCATCATGGAAAGAAGCAGCTTCGAGAAGAATCCCATCGACT
TTCTGGAAGCCAAGGGCTACAAAGAAGTGAAAAAGGACCTGATCATCAAGCT
GCCTAAGTACTCCCTGTTCGAGCTGGAAAACGGCCGGAAGAGAATGCTGGCCT
CTGCCGGCGAACTGCAGAAGGGAAACGAACTGGCCCTGCCCTCCAAATATGTG
AACTTCCTGTACCTGGCCAGCCACTATGAGAAGCTGAAGGGCTCCCCCGAGGA
TAATGAGCAGAAACAGCTGTTTGTGGAACAGCACAAACACTACCTGGACGAG
ATCATCGAGCAGATCAGCGAGTTCTCCAAGAGAGTGATCCTGGCCGACGCTAA
TCTGGACAAGGTGCTGAGCGCCTACAACAAGCACAGAGACAAGCCTATCAGA
GAGCAGGCCGAGAATATCATCCACCTGTTTACCCTGACCAATCTGGGAGCCCC
TGCCGCCTTCAAGTACTTTGACACCACCATCGACCGGAAGAGGTACACCAGCA
CCAAAGAGGTGCTGGACGCCACCCTGATCCACCAGAGCATCACCGGCCTGTAC
GAGACACGGATCGACCTGTCTCAGCTGGGAGGCGACGCCTATCCCTATGACGT
GCCCGATTATGCCAGCCTGGGCAGCGGCTCCCCCAAGAAAAAACGCAAGGTG
GAAGGATCCGGCGCAACAAACTTCTCTCTGCTGAAACAAGCCGGAGATGTCGA
AGAGAATCCTGGACCGATGGCCAAGCCTTTGTCTCAAGAAGAATCCACCCTCA
TTGAAAGAGCAACGGCTACAATCAACAGCATCCCCATCTCTGAAGACTACAGC
GTCGCCAGCGCAGCTCTCTCTAGCGACGGCCGCATCTTCACTGGTGTCAATGTA
TATCATTTTACTGGGGGACCTTGTGCAGAACTCGTGGTGCTGGGCACTGCTGCT
GCTGCGGCAGCTGGCAACCTGACTTGTATCGTCGCGATCGGAAATGAGAACAG
GGGCATCTTGAGCCCCTGCGGACGGTGCCGACAGGTGCTTCTCGATCTGCATC
CTGGGATCAAAGCCATAGTGAAGGACAGTGATGGACAGCCGACGGCAGTTGG
GATTCGTGAATTGCTGCCCTCTGGTTATGTGTGGGAGGGCTAAGAATTCAATCA
ACCTCTGGATTACAAAATTTGTGAAAGATTGACTGGTATTCTTAACTATGTTGC
TCCTTTTACGCTATGTGGATACGCTGCTTTAATGCCTTTGTATCATGCTATTGCT
TCCCGTATGGCTTTCATTTTCTCCTCCTTGTATAAATCCTGGTTGCTGTCTCTTT
ATGAGGAGTTGTGGCCCGTTGTCAGGCAACGTGGCGTGGTGTGCACTGTGTTT
GCTGACGCAACCCCCACTGGTTGGGGCATTGCCACCACCTGTCAGCTCCTTTCC
GGGACTTTCGCTTTCCCCCTCCCTATTGCCACGGCGGAACTCATCGCCGCCTGC
CTTGCCCGCTGCTGGACAGGGGCTCGGCTGTTGGGCACTGACAATTCCGTGGT
GTTGTCGGGGAAGCTGACGTCCTTTCCATGGCTGCTCGCCTGTGTTGCCACCTG
GATTCTGCGCGGGACGTCCTTCTGCTACGTCCCTTCGGCCCTCAATCCAGCGGA
CCTTCCTTCCCGCGGCCTGCTGCCGGCTCTGCGGCCTCTTCCGCGTCTTCGCCTT
CGCCCTCAGACGAGTCGGATCTCCCTTTGGGCCGCCTCCCCGCGAGACGTTTCA
TTTCCGTCTCTGGTACCACTTTTTAAAAGAAAAGGGGGGACTGGAAGGGCTAA
TTCACTCCCAACGAAGTCAAGATATCCTTGATCTGTGGATCGTTAACTACCACA
CACAAGGCTACTTCCCTGATTGGCAGAACTACACACCAGGGCCAGGGATCAAA
TATCCACTGACCTTTGGATGGTGCTACAAGCTAGTACCAGTTGAGCAAGAGAA
GGTAGAAGAAGCCAATGAAGGAGAGAACACCCGCTTGTTACACCCTGTGAGC
CTGCATGGGATGGATGACCCGGAGAGAGAAGTATTAGAGTGGAGGGTTAACTT
AATTAAGACAGCCGCCTAGCATTTCATCACATGGCCCGAGAGCTGCATCCGGA
CTGTACTGGCTCTCTCTGGTTAGACCAGATCTGAGCCTGGGAGCTCTCTGGCTA
ACTAGGGAACCCACTGCTTAAGCCTCAATAAAGCTTGCCTTGAGTGCTTCAAG
TAGTGTGTGCCCGTCTGTTGTGTGACTCTGGTAACTAGAGATCCCTCAGACCCT
TTTAGTCAGTGTGGAAAATCTCTAGCAGGGCCCTCTAGAGTTTAAACCCGCTG
ATCAGCCTCGACTGTGCCTTCTAGTTGCCAGCCATCTGTTGTTTGCCCCTCCCCC
GTGCCTTCCTTGACCCTGGAAGGTGCCACTCCCACTGTCCTTTCCTAATAAAAT
GAGGAAATTGCATCGCATTGTCTGAGTAGGTGTCATTCTATTCTGGGGGGTGG
GGTGGGGCAGGACAGCAAGGGGGAGGATTGGGAAGAGAATAGCAGGCATGCT
GGGGATGCGGTGGGCTCTATGGCTTCTGAGGCGGAAAGAACCAGCTGGGGCTC
TAGGGGGTATCCCCACGCGCCCTGTAGCGGCGCATTAAGCGCGGCGGGTGTGG
TGGTTACGCGCAGCGTGACCGCTACACTTGCCAGCGCCCTAGCGCCCGCTCCTT
TCGCTTTCTTCCCTTCCTTTCTCGCCACGTTCGCCGGCTTTCCCCGTCAAGCTCT
AAATCGGGGGCTCCCTTTAGGGTTCCGATTTAGTGCTTTACGGCACCTCGACCC
CAAAAAACTTGATTAGGGTGATGGTTCACGTAGTGGGCCATCGCCCTGATAGA
CGGTTTTTCGCCCTTTGACGTTGGAGTCCACGTTCTTTAATAGTGGACTCTTGTT
CCAAACTGGAACAACACTCAACCCTATCTCGGTCTATTCTTTTGATTTATAAGG
GATTTTGCCGATTTCGGCCTATTGGTTAAAAAATGAGCTGATTTAACAAAAATT
TAACGCGAATTAATTCTGTGGAATGTGTGTCAGTTAGGGTGTGGAAAGTCCCC
AGGCTCCCCAGCAGGCAGAAGTATGCAAAGCATGCATCTCAATTAGTCAGCAA
CCAGGTGTGGAAAGTCCCCAGGCTCCCCAGCAGGCAGAAGTATGCAAAGCAT
GCATCTCAATTAGTCAGCAACCATAGTCCCGCCCCTAACTCCGCCCATCCCGCC
CCTAACTCCGCCCAGTTCCGCCCATTCTCCGCCCCATGGCTGACTAATTTTTTTT
ATTTATGCAGAGGCCGAGGCCGCCTCTGCCTCTGAGCTATTCCAGAAGTAGTG
AGGAGGCTTTTTTGGAGGCCTAGGCTTTTGCAAAAAGCTCCCGGGAGCTTGTA
TATCCATTTTCGGATCTGATCAGCACGTGTTGACAATTAATCATCGGCATAGTA
TATCGGCATAGTATAATACGACAAGGTGAGGAACTAAACCATGGCCAAGTTGA
CCAGTGCCGTTCCGGTGCTCACCGCGCGCGACGTCGCCGGAGCGGTCGAGTTC
TGGACCGACCGGCTCGGGTTCTCCCGGGACTTCGTGGAGGACGACTTCGCCGG
TGTGGTCCGGGACGACGTGACCCTGTTCATCAGCGCGGTCCAGGACCAGGTGG
TGCCGGACAACACCCTGGCCTGGGTGTGGGTGCGCGGCCTGGACGAGCTGTAC
GCCGAGTGGTCGGAGGTCGTGTCCACGAACTTCCGGGACGCCTCCGGGCCGGC
CATGACCGAGATCGGCGAGCAGCCGTGGGGGCGGGAGTTCGCCCTGCGCGAC
CCGGCCGGCAACTGCGTGCACTTCGTGGCCGAGGAGCAGGACTGACACGTGCT
ACGAGATTTCGATTCCACCGCCGCCTTCTATGAAAGGTTGGGCTTCGGAATCGT
TTTCCGGGACGCCGGCTGGATGATCCTCCAGCGCGGGGATCTCATGCTGGAGT
TCTTCGCCCACCCCAACTTGTTTATTGCAGCTTATAATGGTTACAAATAAAGCA
ATAGCATCACAAATTTCACAAATAAAGCATTTTTTTCACTGCATTCTAGTTGTG
GTTTGTCCAAACTCATCAATGTATCTTATCATGTCTGTATACCGTCGACCTCTA
GCTAGAGCTTGGCGTAATCATGGTCATAGCTGTTTCCTGTGTGAAATTGTTATC
CGCTCACAATTCCACACAACATACGAGCCGGAAGCATAAAGTGTAAAGCCTGG
GGTGCCTAATGAGTGAGCTAACTCACATTAATTGCGTTGCGCTCACTGCCCGCT
TTCCAGTCGGGAAACCTGTCGTGCCAGCTGCATTAATGAATCGGCCAACGCGC
GGGGAGAGGCGGTTTGCGTATTGGGCGCTCTTCCGCTTCCTCGCTCACTGACTC
GCTGCGCTCGGTCGTTCGGCTGCGGCGAGCGGTATCAGCTCACTCAAAGGCGG
TAATACGGTTATCCACAGAATCAGGGGATAACGCAGGAAAGAACATGTGAGC
AAAAGGCCAGCAAAAGGCCAGGAACCGTAAAAAGGCCGCGTTGCTGGCGTTT
TTCCATAGGCTCCGCCCCCCTGACGAGCATCACAAAAATCGACGCTCAAGTCA
GAGGTGGCGAAACCCGACAGGACTATAAAGATACCAGGCGTTTCCCCCTGGAA
GCTCCCTCGTGCGCTCTCCTGTTCCGACCCTGCCGCTTACCGGATACCTGTCCG
CCTTTCTCCCTTCGGGAAGCGTGGCGCTTTCTCATAGCTCACGCTGTAGGTATC
TCAGTTCGGTGTAGGTCGTTCGCTCCAAGCTGGGCTGTGTGCACGAACCCCCCG
TTCAGCCCGACCGCTGCGCCTTATCCGGTAACTATCGTCTTGAGTCCAACCCGG
TAAGACACGACTTATCGCCACTGGCAGCAGCCACTGGTAACAGGATTAGCAGA
GCGAGGTATGTAGGCGGTGCTACAGAGTTCTTGAAGTGGTGGCCTAACTACGG
CTACACTAGAAGAACAGTATTTGGTATCTGCGCTCTGCTGAAGCCAGTTACCTT
CGGAAAAAGAGTTGGTAGCTCTTGATCCGGCAAACAAACCACCGCTGGTAGCG
GTGGTTTTTTTGTTTGCAAGCAGCAGATTACGCGCAGAAAAAAAGGATCTCAA
GAAGATCCTTTGATCTTTTCTACGGGGTCTGACGCTCAGTGGAACGAAAACTC
ACGTTAAGGGATTTTGGTCATGAGATTATCAAAAAGGATCTTCACCTAGATCCT
TTTAAATTAAAAATGAAGTTTTAAATCAATCTAAAGTATATATGAGTAAACTTG
GTCTGACAGTTACCAATGCTTAATCAGTGAGGCACCTATCTCAGCGATCTGTCT
ATTTCGTTCATCCATAGTTGCCTGACTCCCCGTCGTGTAGATAACTACGATACG
GGAGGGCTTACCATCTGGCCCCAGTGCTGCAATGATACCGCGGGACCCACGCT
CACCGGCTCCAGATTTATCAGCAATAAACCAGCCAGCCGGAAGGGCCGAGCGC
AGAAGTGGTCCTGCAACTTTATCCGCCTCCATCCAGTCTATTAATTGTTGCCGG
GAAGCTAGAGTAAGTAGTTCGCCAGTTAATAGTTTGCGCAACGTTGTTGCCATT
GCTACAGGCATCGTGGTGTCACGCTCGTCGTTTGGTATGGCTTCATTCAGCTCC
GGTTCCCAACGATCAAGGCGAGTTACATGATCCCCCATGTTGTGCAAAAAAGC
GGTTAGCTCCTTCGGTCCTCCGATCGTTGTCAGAAGTAAGTTGGCCGCAGTGTT
ATCACTCATGGTTATGGCAGCACTGCATAATTCTCTTACTGTCATGCCATCCGT
AAGATGCTTTTCTGTGACTGGTGAGTACTCAACCAAGTCATTCTGAGAATAGTG
TATGCGGCGACCGAGTTGCTCTTGCCCGGCGTCAATACGGGATAATACCGCGC
CACATAGCAGAACTTTAAAAGTGCTCATCATTGGAAAACGTTCTTCGGGGCGA
AAACTCTCAAGGATCTTACCGCTGTTGAGATCCAGTTCGATGTAACCCACTCGT
GCACCCAACTGATCTTCAGCATCTTTTACTTTCACCAGCGTTTCTGGGTGAGCA
AAAACAGGAAGGCAAAATGCCGCAAAAAAGGGAATAAGGGCGACACGGAAA
TGTTGAATACTCATACTCTTCCTTTTTCAATATTATTGAAGCATTTATCAGGGTT
ATTGTCTCATGAGCGGATACATATTTGAATGTATTTAGAAAAATAAACAAATA
GGGGTTCCGCGCACATTTCCCCGAAAAGTGCCACCTGACGTCGACGGATCGGG
AGATCTCCCGATCCCCTATGGTGCACTCTCAGTACAATCTGCTCTGATGCCGCA
TAGTTAAGCCAGTATCTGCTCCCTGCTTGTGTGTTGGAGGTCGCTGAGTAGTGC
GCGAGCAAAATTTAAGCTACAACAAGGCAAGGCTTGACCGACAATTGCATGA
AGAATCTGCTTAGGGTTAGGCGTTTTGCGCTGCTTCGCGATGTACGGGCCAGAT
ATACGCGTTGACATTGATTATTGACTAGTTATTAATAGTAATCAATTACGGGGT
CATTAGTTCATAGCCCATATATGGAGTTCCGCGTTACATAACTTACGGTAAATG
GCCCGCCTGGCTGACCGCCCAACGACCCCCGCCCATTGACGTCAATAATGACG
TATGTTCCCATAGTAACGCCAATAGGGACTTTCCATTGACGTCAATGGGTGGA
GTATTTACGGTAAACTGCCCACTTGGCAGTACATCAAGTGTATCATATGCCAA
GTACGCCCCCTATTGACGTCAATGACGGTAAATGGCCCGCCTGGCATTATGCC
CAGTACATGACCTTATGGGACTTTCCTACTTGGCAGTACATCTACGTATTAGTC
ATCGCTATTACCATGGTGATGCGGTTTTGGCAGTACATCAATGGGCGTGGATA
GCGGTTTGACTCACGGGGATTTCCAAGTCTCCACCCCATTGACGTCAATGGGA
GTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGC
AGCGCGTTTTGCCTGTACTGGCTCTCTCTGGTTAGACCAGATCTGAGCCTGGGA
GCTCTCTGGCTAACTAGGGAACCCACTGCTTAAGCCTCAATAAAGCTTGCCTTG
AGTGCTTCAAGTAGTGTGTGCCCGTCTGTTGTGTGACTCTGGTAACTAGAGATC
CCTCAGACCCTTTTAGTCAGTGTGGAAAATCTCTAGCAGTGGCGCCCGAACAG
GGACTTGAAAGCGAAAGGGAAACCAGAGGAGCTCTCTCGACGCAGGACTCGG
CTTGCTGAAGCGCGCACGGCAAGAGGCGAGGGGCGGCGACTGGTGAGTACGC
CAAAAATTTTGACTAGCGGAGGCTAGAAGGAGAGAGATGGGTGCGAGAGCGT
CAGTATTAAGCGGGGGAGAATTAGATCGCGATGGGAAAAAATTCGGTTAAGG
CCAGGGGGAAAGAAAAAATATAAATTAAAACATATAGTATGGGCAAGCAGGG
AGCTAGAACGATTCGCAGTTAATCCTGGCCTGTTAGAAACATCAGAAGGCTGT
AGACAAATACTGGGACAGCTACAACCATCCCTTCAGACAGGATCAGAAGAACT
TAGATCATTATATAATACAGTAGCAACCCTCTATTGTGTGCATCAAAGGATAG
AGATAAAAGACACCAAGGAAGCTTTAGACAAGATAGAGGAAGAGCAAAACAA
AAGTAAGACCACCGCACAGCAAGCGGCCGCTGATCTTCAGACCTGGAGGAGG
AGATATGAGGGACAATTGGAGAAGTGAATTATATAAATATAAAGTAGTAAAA
ATTGAACCATTAGGAGTAGCACCCACCAAGGCAAAGAGAAGAGTGGTGCAGA
GAGAAAAAAGAGCAGTGGGAATAGGAGCTTTGTTCCTTGGGTTCTTGGGAGCA
GCAGGAAGCACTATGGGCGCAGCGTCAATGACGCTGACGGTACAGGCCAGAC
AATTATTGTCTGGTATAGTGCAGCAGCAGAACAATTTGCTGAGGGCTATTGAG
GCGCAACAGCATCTGTTGCAACTCACAGTCTGGGGCATCAAGCAGCTCCAGGC
AAGAATCCTGGCTGTGGAAAGATACCTAAAGGATCAACAGCTCCTGGGGATTT
GGGGTTGCTCTGGAAAACTCATTTGCACCACTGCTGTGCCTTGGAATGCTAGTT
GGAGTAATAAATCTCTGGAACAGATTTGGAATCACACGACCTGGATGGAGTGG
GACAGAGAAATTAACAATTACACAAGCTTAATACACTCCTTAATTGAAGAATC
GCAAAACCAGCAAGAAAAGAATGAACAAGAATTATTGGAATTAGATAAATGG
GCAAGTTTGTGGAATTGGTTTAACATAACAAATTGGCTGTGGTATATAAAATT
ATTCATAATGATAGTAGGAGGCTTGGTAGGTTTAAGAATAGTTTTTGCTGTACT
TTCTATAGTGAATAGAGTTAGGCAGGGATATTCACCATTATCGTTTCAGACCCA
CCTCCCAACCCCGAGGGGACCCGACAGGCCCGAAGGAATAGAAGAAGAAGGT
GGAGAGAGAGACAGAGACAGATCCATTCGATTAGTGAACGGATCGGCACTGC
GTGCGCCAATTCTGCAGACAAATGGCAGTATTCATCCACAATTTTAAAAGAAA
AGGGGGGATTGGGGGGTACAGTGCAGGGGAAAGAATAGTAGACATAATAGCA
ACAGACATACAAACTAAAGAATTACAAAAACAAATTACAAAAATTCAAAATTT
TCGGGTTTATTACAGGGACAGCAGAGATCCAGTTTGGTTAATTAGGGTTAATT
AGCTAGCGGCTCCGGTGCCCGTCAGTGGGCAGAGCGCACATCGCCCACAGTCC
CCGAGAAGTTGGGGGGAGGGGTCGGCAATTGATCCGGTGCCTAGAGAAGGTG
GCGCGGGGTAAACTGGGAAAGTGATGTCGTGTACTGGCTCCGCCTTTTTCCCG
AGGGTGGGGGAGAACCGTATATAAGTGCAGTAGTCGCCGTGAACGTTCTTTTT
CGCAACGGGTTTGCCGCCAGAACACAGGC
SEQ ID NO: 6-DNA Sequence of the IDT fragment #1 and #2 in lentiviral
vector
GATCCAGTAAACGACCTGCCGCCACTAAAAAAGCCGGACAGGCTAAGAAGAA
GAAAGGAGGTTCAGGAGGATCTGGGGGGAGCGGAGGGAGCATGTCCAGGCGG
AAACAGAGCAACCCCCGGCAGATCAAGCGTTCCCTCGGAGACATGGAGGCCA
GAGAGGAGGTGCAGTTGGTGGGTGCCAGCCACATGGAGCAAAAGGCCACGGC
ACCTGAAGCCCCGAGCCCTGGCTCCGGCGCAACAAACTTCTCTCTGCTGAAAC
AAGCCGGAGATGTCGAAGAGAATCCTGGACCGATGGCCAAGCCTTTGTCTCAA
GAAGAATCCACCCTCATTGAAAGAGCAACGGCTACAATCAACAGCATCCCCAT
CTCTGAAGACTACAGCGTCGCCAGCGCAGCTCTCTCTAGCGACGGCCGCATCT
TCACTGGTGTCAATGTATATCATTTTACTGGGGGACCTTGTGCAGAACTCGTGG
TGCTGGGCACTGCTGCTGCTGCGGCAGCTGGCAACCTGACTTGTATCGTCGCG
ATCGGAAATGAGAACAGGGGCATCTTGAGCCCCTGCGGACGGTGCCGACAGG
TGCTTCTCGATCTGCATCCTGGGATCAAAGCCATAGTGAAGGACAGTGATGGA
CAGCCGACGGCAGTTGGGATTCGTGAATTGCTGCCCTCTGGTTATGTGTGGGA
GGGCTAAGAATTCAATCAACCTCTGGATTACAAAATTTGTGAAAGATTGACTG
GTATTCTTAACTATGTTGCTCCTTTTACGCTATGTGGATACGCTGCTTTAATGCC
TTTGTATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACGTGGC
GTGGTGTGCACTGTGTTTGCTGACGCAACCCCCACTGGTTGGGGCATTGCCACC
ACCTGTCAGCTCCTTTCCGGGACTTTCGCTTTCCCCCTCCCTATTGCCACGGCG
GAACTCATCGCCGCCTGCCTTGCCCGCTGCTGGACAGGGGCTCGGCTGTTGGG
CACTGACAATTCCGTGGTGTTGTCGGGGAAGCTGACGTCCTTTCCATGGCTGCT
CGCCTGTGTTGCCACCTGGATTCTGCGCGGGACGTCCTTCTGCTACGTCCCTTC
GGCCCTCAATCCAGCGGACCTTCCTTCCCGCGGCCTGCTGCCGGCTCTGCGGCC
TCTTCCGCGTCTTCGCCTTCGCCCTCAGACGAGTCGGATCTCCCTTTGGGCCGC
CTCCCCGCGAGACGTTTCATTTCCGTCTCTGGTACCACTTTTTAAAAGAAAAGG
GGGGACTGGAAGGGCTAATTCACTCCCAACGAAGTCAAGATATCCTTGATCTG
TGGATCGTTAACTACCACACACAAGGCTACTTCCCTGATTGGCAGAACTACAC
ACCAGGGCCAGGGATCAAATATCCACTGACCTTTGGATGGTGCTACAAGCTAG
TACCAGTTGAGCAAGAGAAGGTAGAAGAAGCCAATGAAGGAGAGAACACCCG
CTTGTTACACCCTGTGAGCCTGCATGGGATGGATGACCCGGAGAGAGAAGTAT
TAGAGTGGAGGGTTAACTTAATTAAGACAGCCGCCTAGCATTTCATCACATGG
CCCGAGAGCTGCATCCGGACTGTACTGGCTCTCTCTGGTTAGACCAGATCTGA
GCCTGGGAGCTCTCTGGCTAACTAGGGAACCCACTGCTTAAGCCTCAATAAAG
CTTGCCTTGAGTGCTTCAAGTAGTGTGTGCCCGTCTGTTGTGTGACTCTGGTAA
CTAGAGATCCCTCAGACCCTTTTAGTCAGTGTGGAAAATCTCTAGCAGGGCCCT
CTAGAGTTTAAACCCGCTGATCAGCCTCGACTGTGCCTTCTAGTTGCCAGCCAT
CTGTTGTTTGCCCCTCCCCCGTGCCTTCCTTGACCCTGGAAGGTGCCACTCCCA
CTGTCCTTTCCTAATAAAATGAGGAAATTGCATCGCATTGTCTGAGTAGGTGTC
ATTCTATTCTGGGGGGTGGGGTGGGGCAGGACAGCAAGGGGGAGGATTGGGA
AGAGAATAGCAGGCATGCTGGGGATGCGGTGGGCTCTATGGCTTCTGAGGCGG
AAAGAACCAGCTGGGGCTCTAGGGGGTATCCCCACGCGCCCTGTAGCGGCGCA
TTAAGCGCGGCGGGTGTGGTGGTTACGCGCAGCGTGACCGCTACACTTGCCAG
CGCCCTAGCGCCCGCTCCTTTCGCTTTCTTCCCTTCCTTTCTCGCCACGTTCGCC
GGCTTTCCCCGTCAAGCTCTAAATCGGGGGCTCCCTTTAGGGTTCCGATTTAGT
GCTTTACGGCACCTCGACCCCAAAAAACTTGATTAGGGTGATGGTTCACGTAG
TGGGCCATCGCCCTGATAGACGGTTTTTCGCCCTTTGACGTTGGAGTCCACGTT
CTTTAATAGTGGACTCTTGTTCCAAACTGGAACAACACTCAACCCTATCTCGGT
CTATTCTTTTGATTTATAAGGGATTTTGCCGATTTCGGCCTATTGGTTAAAAAA
TGAGCTGATTTAACAAAAATTTAACGCGAATTAATTCTGTGGAATGTGTGTCA
GTTAGGGTGTGGAAAGTCCCCAGGCTCCCCAGCAGGCAGAAGTATGCAAAGC
ATGCATCTCAATTAGTCAGCAACCAGGTGTGGAAAGTCCCCAGGCTCCCCAGC
AGGCAGAAGTATGCAAAGCATGCATCTCAATTAGTCAGCAACCATAGTCCCGC
CCCTAACTCCGCCCATCCCGCCCCTAACTCCGCCCAGTTCCGCCCATTCTCCGC
CCCATGGCTGACTAATTTTTTTTATTTATGCAGAGGCCGAGGCCGCCTCTGCCT
CTGAGCTATTCCAGAAGTAGTGAGGAGGCTTTTTTGGAGGCCTAGGCTTTTGCA
AAAAGCTCCCGGGAGCTTGTATATCCATTTTCGGATCTGATCAGCACGTGTTGA
CAATTAATCATCGGCATAGTATATCGGCATAGTATAATACGACAAGGTGAGGA
ACTAAACCATGGCCAAGTTGACCAGTGCCGTTCCGGTGCTCACCGCGCGCGAC
GTCGCCGGAGCGGTCGAGTTCTGGACCGACCGGCTCGGGTTCTCCCGGGACTT
CGTGGAGGACGACTTCGCCGGTGTGGTCCGGGACGACGTGACCCTGTTCATCA
GCGCGGTCCAGGACCAGGTGGTGCCGGACAACACCCTGGCCTGGGTGTGGGTG
CGCGGCCTGGACGAGCTGTACGCCGAGTGGTCGGAGGTCGTGTCCACGAACTT
CCGGGACGCCTCCGGGCCGGCCATGACCGAGATCGGCGAGCAGCCGTGGGGG
CGGGAGTTCGCCCTGCGCGACCCGGCCGGCAACTGCGTGCACTTCGTGGCCGA
GGAGCAGGACTGACACGTGCTACGAGATTTCGATTCCACCGCCGCCTTCTATG
AAAGGTTGGGCTTCGGAATCGTTTTCCGGGACGCCGGCTGGATGATCCTCCAG
CGCGGGGATCTCATGCTGGAGTTCTTCGCCCACCCCAACTTGTTTATTGCAGCT
TATAATGGTTACAAATAAAGCAATAGCATCACAAATTTCACAAATAAAGCATT
TTTTTCACTGCATTCTAGTTGTGGTTTGTCCAAACTCATCAATGTATCTTATCAT
GTCTGTATACCGTCGACCTCTAGCTAGAGCTTGGCGTAATCATGGTCATAGCTG
TTTCCTGTGTGAAATTGTTATCCGCTCACAATTCCACACAACATACGAGCCGGA
AGCATAAAGTGTAAAGCCTGGGGTGCCTAATGAGTGAGCTAACTCACATTAAT
TGCGTTGCGCTCACTGCCCGCTTTCCAGTCGGGAAACCTGTCGTGCCAGCTGCA
TTAATGAATCGGCCAACGCGCGGGGAGAGGCGGTTTGCGTATTGGGCGCTCTT
CCGCTTCCTCGCTCACTGACTCGCTGCGCTCGGTCGTTCGGCTGCGGCGAGCGG
TATCAGCTCACTCAAAGGCGGTAATACGGTTATCCACAGAATCAGGGGATAAC
GCAGGAAAGAACATGTGAGCAAAAGGCCAGCAAAAGGCCAGGAACCGTAAA
AAGGCCGCGTTGCTGGCGTTTTTCCATAGGCTCCGCCCCCCTGACGAGCATCAC
AAAAATCGACGCTCAAGTCAGAGGTGGCGAAACCCGACAGGACTATAAAGAT
ACCAGGCGTTTCCCCCTGGAAGCTCCCTCGTGCGCTCTCCTGTTCCGACCCTGC
CGCTTACCGGATACCTGTCCGCCTTTCTCCCTTCGGGAAGCGTGGCGCTTTCTC
ATAGCTCACGCTGTAGGTATCTCAGTTCGGTGTAGGTCGTTCGCTCCAAGCTGG
GCTGTGTGCACGAACCCCCCGTTCAGCCCGACCGCTGCGCCTTATCCGGTAACT
ATCGTCTTGAGTCCAACCCGGTAAGACACGACTTATCGCCACTGGCAGCAGCC
ACTGGTAACAGGATTAGCAGAGCGAGGTATGTAGGCGGTGCTACAGAGTTCTT
GAAGTGGTGGCCTAACTACGGCTACACTAGAAGAACAGTATTTGGTATCTGCG
CTCTGCTGAAGCCAGTTACCTTCGGAAAAAGAGTTGGTAGCTCTTGATCCGGC
AAACAAACCACCGCTGGTAGCGGTGGTTTTTTTGTTTGCAAGCAGCAGATTAC
GCGCAGAAAAAAAGGATCTCAAGAAGATCCTTTGATCTTTTCTACGGGGTCTG
ACGCTCAGTGGAACGAAAACTCACGTTAAGGGATTTTGGTCATGAGATTATCA
AAAAGGATCTTCACCTAGATCCTTTTAAATTAAAAATGAAGTTTTAAATCAATC
TAAAGTATATATGAGTAAACTTGGTCTGACAGTTACCAATGCTTAATCAGTGA
GGCACCTATCTCAGCGATCTGTCTATTTCGTTCATCCATAGTTGCCTGACTCCC
CGTCGTGTAGATAACTACGATACGGGAGGGCTTACCATCTGGCCCCAGTGCTG
CAATGATACCGCGGGACCCACGCTCACCGGCTCCAGATTTATCAGCAATAAAC
CAGCCAGCCGGAAGGGCCGAGCGCAGAAGTGGTCCTGCAACTTTATCCGCCTC
CATCCAGTCTATTAATTGTTGCCGGGAAGCTAGAGTAAGTAGTTCGCCAGTTA
ATAGTTTGCGCAACGTTGTTGCCATTGCTACAGGCATCGTGGTGTCACGCTCGT
CGTTTGGTATGGCTTCATTCAGCTCCGGTTCCCAACGATCAAGGCGAGTTACAT
GATCCCCCATGTTGTGCAAAAAAGCGGTTAGCTCCTTCGGTCCTCCGATCGTTG
TCAGAAGTAAGTTGGCCGCAGTGTTATCACTCATGGTTATGGCAGCACTGCAT
AATTCTCTTACTGTCATGCCATCCGTAAGATGCTTTTCTGTGACTGGTGAGTAC
TCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCA
TCATTGGAAAACGTTCTTCGGGGCGAAAACTCTCAAGGATCTTACCGCTGTTG
AGATCCAGTTCGATGTAACCCACTCGTGCACCCAACTGATCTTCAGCATCTTTT
ACTTTCACCAGCGTTTCTGGGTGAGCAAAAACAGGAAGGCAAAATGCCGCAAA
AAAGGGAATAAGGGCGACACGGAAATGTTGAATACTCATACTCTTCCTTTTTC
AATATTATTGAAGCATTTATCAGGGTTATTGTCTCATGAGCGGATACATATTTG
AATGTATTTAGAAAAATAAACAAATAGGGGTTCCGCGCACATTTCCCCGAAAA
GTGCCACCTGACGTCGACGGATCGGGAGATCTCCCGATCCCCTATGGTGCACT
CTCAGTACAATCTGCTCTGATGCCGCATAGTTAAGCCAGTATCTGCTCCCTGCT
TGTGTGTTGGAGGTCGCTGAGTAGTGCGCGAGCAAAATTTAAGCTACAACAAG
GCAAGGCTTGACCGACAATTGCATGAAGAATCTGCTTAGGGTTAGGCGTTTTG
CGCTGCTTCGCGATGTACGGGCCAGATATACGCGTTGACATTGATTATTGACTA
GTTATTAATAGTAATCAATTACGGGGTCATTAGTTCATAGCCCATATATGGAGT
TCCGCGTTACATAACTTACGGTAAATGGCCCGCCTGGCTGACCGCCCAACGAC
CCCCGCCCATTGACGTCAATAATGACGTATGTTCCCATAGTAACGCCAATAGG
GACTTTCCATTGACGTCAATGGGTGGAGTATTTACGGTAAACTGCCCACTTGGC
AGTACATCAAGTGTATCATATGCCAAGTACGCCCCCTATTGACGTCAATGACG
GTAAATGGCCCGCCTGGCATTATGCCCAGTACATGACCTTATGGGACTTTCCTA
CTTGGCAGTACATCTACGTATTAGTCATCGCTATTACCATGGTGATGCGGTTTT
GGCAGTACATCAATGGGCGTGGATAGCGGTTTGACTCACGGGGATTTCCAAGT
CTCCACCCCATTGACGTCAATGGGAGTTTGTTTTGGCACCAAAATCAACGGGA
CTTTCCAAAATGTCGTAACAACTCCGCCCCATTGACGCAAATGGGCGGTAGGC
GTGTACGGTGGGAGGTCTATATAAGCAGCGCGTTTTGCCTGTACTGGCTCTCTC
TGGTTAGACCAGATCTGAGCCTGGGAGCTCTCTGGCTAACTAGGGAACCCACT
GCTTAAGCCTCAATAAAGCTTGCCTTGAGTGCTTCAAGTAGTGTGTGCCCGTCT
GTTGTGTGACTCTGGTAACTAGAGATCCCTCAGACCCTTTTAGTCAGTGTGGAA
AATCTCTAGCAGTGGCGCCCGAACAGGGACTTGAAAGCGAAAGGGAAACCAG
AGGAGCTCTCTCGACGCAGGACTCGGCTTGCTGAAGCGCGCACGGCAAGAGGC
GAGGGGCGGCGACTGGTGAGTACGCCAAAAATTTTGACTAGCGGAGGCTAGA
AGGAGAGAGATGGGTGCGAGAGCGTCAGTATTAAGCGGGGGAGAATTAGATC
GCGATGGGAAAAAATTCGGTTAAGGCCAGGGGGAAAGAAAAAATATAAATTA
AAACATATAGTATGGGCAAGCAGGGAGCTAGAACGATTCGCAGTTAATCCTGG
CCTGTTAGAAACATCAGAAGGCTGTAGACAAATACTGGGACAGCTACAACCAT
CCCTTCAGACAGGATCAGAAGAACTTAGATCATTATATAATACAGTAGCAACC
CTCTATTGTGTGCATCAAAGGATAGAGATAAAAGACACCAAGGAAGCTTTAGA
CAAGATAGAGGAAGAGCAAAACAAAAGTAAGACCACCGCACAGCAAGCGGCC
GCTGATCTTCAGACCTGGAGGAGGAGATATGAGGGACAATTGGAGAAGTGAA
TTATATAAATATAAAGTAGTAAAAATTGAACCATTAGGAGTAGCACCCACCAA
GGCAAAGAGAAGAGTGGTGCAGAGAGAAAAAAGAGCAGTGGGAATAGGAGC
TTTGTTCCTTGGGTTCTTGGGAGCAGCAGGAAGCACTATGGGCGCAGCGTCAA
TGACGCTGACGGTACAGGCCAGACAATTATTGTCTGGTATAGTGCAGCAGCAG
AACAATTTGCTGAGGGCTATTGAGGCGCAACAGCATCTGTTGCAACTCACAGT
CTGGGGCATCAAGCAGCTCCAGGCAAGAATCCTGGCTGTGGAAAGATACCTAA
AGGATCAACAGCTCCTGGGGATTTGGGGTTGCTCTGGAAAACTCATTTGCACC
ACTGCTGTGCCTTGGAATGCTAGTTGGAGTAATAAATCTCTGGAACAGATTTG
GAATCACACGACCTGGATGGAGTGGGACAGAGAAATTAACAATTACACAAGC
TTAATACACTCCTTAATTGAAGAATCGCAAAACCAGCAAGAAAAGAATGAACA
AGAATTATTGGAATTAGATAAATGGGCAAGTTTGTGGAATTGGTTTAACATAA
CAAATTGGCTGTGGTATATAAAATTATTCATAATGATAGTAGGAGGCTTGGTA
GGTTTAAGAATAGTTTTTGCTGTACTTTCTATAGTGAATAGAGTTAGGCAGGGA
TATTCACCATTATCGTTTCAGACCCACCTCCCAACCCCGAGGGGACCCGACAG
GCCCGAAGGAATAGAAGAAGAAGGTGGAGAGAGAGACAGAGACAGATCCATT
CGATTAGTGAACGGATCGGCACTGCGTGCGCCAATTCTGCAGACAAATGGCAG
TATTCATCCACAATTTTAAAAGAAAAGGGGGGATTGGGGGGTACAGTGCAGGG
GAAAGAATAGTAGACATAATAGCAACAGACATACAAACTAAAGAATTACAAA
AACAAATTACAAAAATTCAAAATTTTCGGGTTTATTACAGGGACAGCAGAGAT
CCAGTTTGGTTAATTAGGGTTAATTAGCTAGCGGCTCCGGTGCCCGTCAGTGGG
CAGAGCGCACATCGCCCACAGTCCCCGAGAAGTTGGGGGGAGGGGTCGGCAA
TTGATCCGGTGCCTAGAGAAGGTGGCGCGGGGTAAACTGGGAAAGTGATGTCG
TGTACTGGCTCCGCCTTTTTCCCGAGGGTGGGGGAGAACCGTATATAAGTGCA
GTAGTCGCCGTGAACGTTCTTTTTCGCAACGGGTTTGCCGCCAGAACACAGGCT
CGAGGCCACCATGAACAATTCCCAGGGAAGAGTGACCTTCGAGGATGTCACTG
TGAACTTCACCCAGGGGGAGTGGCAGCGGCTGAATCCCGAACAGAGAAACTT
GTACAGGGATGTGATGCTGGAGAATTACAGCAACCTTGTCTCTGTGGGACAAG
GGGAAACCACCAAACCCGATGTGATCTTGAGGTTGGAACAAGGAAAGGAGCC
ATGGTTGGAGGAAGAGGAAGTGCTGGGAAGTGGCCGTGCAGAAAAAAATGGG
GACATTGGAGGGCAGATTTGGAAGCCAAAGGATGTGAAAGAGAGTCTCGGTG
GGTCTGGCGGTTCTGTGCAGGTGAAAAGGGTGCTGGAAAAATCCCCCGGCAAA
CTCCTCGTGAAGATGCCCTTCCAGGCTTCCCCTGGCGGAAAAGGTGAAGGGGG
TGGCGCAACCACATCTGCCCAGGTCATGGTCATCAAGCGACCTGGAAGGAAAA
GAAAGGCCGAGGCTGACCCTCAGGCCATTCCAAAGAAACGGGGACGCAAGCC
AGGGTCCGTGGTCGCAGCTGCAGCAGCTGAGGCTAAGAAAAAGGCAGTGAAG
GAAAGCTCCATCCGCAGTGTGCAGGAGACTGTCCTGCCCATCAAGAAGAGGAA
GACTAGGGAGACCGTGTCCATCGAGGTCAAAGAAGTGGTCAAGCCCCTGCTCG
TGTCCACCCTGGGCGAAAAATCTGGAAAGGGGCTCAAAACATGCAAGTCACCT
GGACGGAAAAGCAAGGAGTCTAGTCCAAAGGGGCGCTCAAGCTCCGCTTCTA
GTCCCCCTAAAAAGGAACACCATCACCATCACCATCACGCCGAGTCTCCTAAG
GCTCCTATGCCACTGCTCCCACCACCTCCACCACCTGAGCCACAGTCAAGCGA
AGACCCCATCAGCCCACCCGAGCCTCAGGATCTGTCCTCTAGTATTTGCAAAG
AGGAAAAGATGCCCAGAGCAGGCAGCCTGGAGAGTGATGGCTGTCCAAAAGA
ACCCGCCAAGACCCAGCCTATGGTGGCAGCCGCTGCAACTACCACCACAACCA
CAACTACCACAGTGGCCGAAAAATACAAGCATCGCGGCGAGGGCGAACGAAA
GGACATTGTGTCAAGCTCCATGCCCAGACCTAACCGGGAGGAACCAGTCGATA
GTAGGACACCCGTGACTGAGAGAGTCTCAGGCTCCGCCGGCAGCGCTGCCGGC
TCAGGGGAGTTTCCTAAGAAAAAGCGGAAAGTGCGTACGGACAAGAAGTACA
GCATCGGCCTGGCCATCGGCACCAACTCTGTGGGCTGGGCCGTGATCACCGAC
GAGTACAAGGTGCCCAGCAAGAAATTCAAGGTGCTGGGCAACACCGACCGGC
ACAGCATCAAGAAGAACCTGATCGGCGCCCTGCTGTTCGACAGCGGAGAAAC
AGCCGAGGCCACCCGGCTGAAGAGAACCGCCAGAAGAAGATACACCAGACGG
AAGAACCGGATCTGCTATCTGCAAGAGATCTTCAGCAACGAGATGGCCAAGGT
GGACGACAGCTTCTTCCACAGACTGGAAGAGTCCTTCCTGGTGGAAGAGGATA
AGAAGCACGAGCGGCACCCCATCTTCGGCAACATCGTGGACGAGGTGGCCTAC
CACGAGAAGTACCCCACCATCTACCACCTGAGAAAGAAACTGGTGGACAGCA
CCGACAAGGCCGACCTGCGGCTGATCTATCTGGCCCTGGCCCACATGATCAAG
TTCCGGGGCCACTTCCTGATCGAGGGCGACCTGAACCCCGACAACAGCGACGT
GGACAAGCTGTTCATCCAGCTGGTGCAGACCTACAACCAGCTGTTCGAGGAAA
ACCCCATCAACGCCAGCGGCGTGGACGCCAAGGCCATCCTGTCTGCCAGACTG
AGCAAGAGCAGACGGCTGGAAAATCTGATCGCCCAGCTGCCCGGCGAGAAGA
AGAATGGCCTGTTCGGCAACCTGATTGCCCTGAGCCTGGGCCTGACCCCCAAC
TTCAAGAGCAACTTCGACCTGGCCGAGGATGCCAAACTGCAGCTGAGCAAGGA
CACCTACGACGACGACCTGGACAACCTGCTGGCCCAGATCGGCGACCAGTACG
CCGACCTGTTTCTGGCCGCCAAGAACCTGTCCGACGCCATCCTGCTGAGCGAC
ATCCTGAGAGTGAACACCGAGATCACCAAGGCCCCCCTGAGCGCCTCTATGAT
CAAGAGATACGACGAGCACCACCAGGACCTGACCCTGCTGAAAGCTCTCGTGC
GGCAGCAGCTGCCTGAGAAGTACAAAGAGATTTTCTTCGACCAGAGCAAGAAC
GGCTACGCCGGCTACATCGATGGCGGAGCCAGCCAGGAAGAGTTCTACAAGTT
CATCAAGCCCATCCTGGAAAAGATGGACGGCACCGAGGAACTGCTCGTGAAG
CTGAACAGAGAGGACCTGCTGCGGAAGCAGCGGACCTTCGACAACGGCAGCA
TCCCCCACCAGATCCACCTGGGAGAGCTGCACGCCATTCTGCGGCGGCAGGAA
GATTTTTACCCATTCCTGAAGGACAACCGGGAAAAGATCGAGAAGATCCTGAC
CTTCCGCATCCCCTACTACGTGGGCCCTCTGGCCAGGGGAAACAGCAGATTCG
CCTGGATGACCAGAAAGAGCGAGGAAACCATCACCCCCTGGAACTTCGAGGA
AGTGGTGGACAAGGGCGCCAGCGCCCAGAGCTTCATCGAGCGGATGACCAAC
TTCGATAAGAACCTGCCCAACGAGAAGGTGCTGCCCAAGCACAGCCTGCTGTA
CGAGTACTTCACCGTGTACAACGAGCTGACCAAAGTGAAATACGTGACCGAGG
GAATGAGAAAGCCCGCCTTCCTGAGCGGCGAGCAGAAAAAAGCCATCGTGGA
CCTGCTGTTCAAGACCAACCGGAAAGTGACCGTGAAGCAGCTGAAAGAGGAC
TACTTCAAGAAAATCGAGTGCTTCGACTCCGTGGAAATCTCCGGCGTGGAAGA
TCGGTTCAACGCCTCCCTGGGCACATACCACGATCTGCTGAAAATTATCAAGG
ACAAGGACTTCCTGGACAATGAGGAAAACGAGGACATTCTGGAAGATATCGT
GCTGACCCTGACACTGTTTGAGGACAGAGAGATGATCGAGGAACGGCTGAAA
ACCTATGCCCACCTGTTCGACGACAAAGTGATGAAGCAGCTGAAGCGGCGGAG
ATACACCGGCTGGGGCAGGCTGAGCCGGAAGCTGATCAACGGCATCCGGGAC
AAGCAGTCCGGCAAGACAATCCTGGATTTCCTGAAGTCCGACGGCTTCGCCAA
CAGAAACTTCATGCAGCTGATCCACGACGACAGCCTGACCTTTAAAGAGGACA
TCCAGAAAGCCCAGGTGTCCGGCCAGGGCGATAGCCTGCACGAGCACATTGCC
AATCTGGCCGGCAGCCCCGCCATTAAGAAGGGCATCCTGCAGACAGTGAAGGT
GGTGGACGAGCTCGTGAAAGTGATGGGCCGGCACAAGCCCGAGAACATCGTG
ATCGAAATGGCCAGAGAGAACCAGACCACCCAGAAGGGACAGAAGAACAGCC
GCGAGAGAATGAAGCGGATCGAAGAGGGCATCAAAGAGCTGGGCAGCCAGAT
CCTGAAAGAACACCCCGTGGAAAACACCCAGCTGCAGAACGAGAAGCTGTAC
CTGTACTACCTGCAGAATGGGCGGGATATGTACGTGGACCAGGAACTGGACAT
CAACCGGCTGTCCGACTACGATGTGGACGCTATCGTGCCTCAGAGCTTTCTGA
AGGACGACTCCATCGATAACAAAGTGCTGACTCGGAGCGACAAGAACCGGGG
CAAGAGCGACAACGTGCCCTCCGAAGAGGTCGTGAAGAAGATGAAGAACTAC
TGGCGCCAGCTGCTGAATGCCAAGCTGATTACCCAGAGGAAGTTCGACAATCT
GACCAAGGCCGAGAGAGGCGGCCTGAGCGAACTGGATAAGGCCGGCTTCATC
AAGAGACAGCTGGTGGAAACCCGGCAGATCACAAAGCACGTGGCACAGATCC
TGGACTCCCGGATGAACACTAAGTACGACGAGAACGACAAACTGATCCGGGA
AGTGAAAGTGATCACCCTGAAGTCCAAGCTGGTGTCCGATTTCCGGAAGGATT
TCCAGTTTTACAAAGTGCGCGAGATCAACAACTACCACCACGCCCACGACGCC
TACCTGAACGCCGTCGTGGGAACCGCCCTGATCAAAAAGTACCCTAAGCTGGA
AAGCGAGTTCGTGTACGGCGACTACAAGGTGTACGACGTGCGGAAGATGATCG
CCAAGAGCGAGCAGGAAATCGGCAAGGCTACCGCCAAGTACTTCTTCTACAGC
AACATCATGAACTTTTTCAAGACCGAGATTACCCTGGCCAACGGCGAGATCCG
GAAGCGGCCTCTGATCGAGACAAACGGCGAAACAGGCGAGATCGTGTGGGAT
AAGGGCCGGGACTTTGCCACCGTGCGGAAAGTGCTGTCTATGCCCCAAGTGAA
TATCGTGAAAAAGACCGAGGTGCAGACAGGCGGCTTCAGCAAAGAGTCTATCC
TGCCCAAGAGGAACAGCGACAAGCTGATCGCCAGAAAGAAGGACTGGGACCC
TAAGAAGTACGGCGGCTTCGACAGCCCCACCGTGGCCTATTCTGTGCTGGTGG
TGGCCAAAGTGGAAAAGGGCAAGTCCAAGAAACTGAAGAGTGTGAAAGAGCT
GCTGGGGATCACCATCATGGAAAGAAGCAGCTTCGAGAAGAATCCCATCGACT
TTCTGGAAGCCAAGGGCTACAAAGAAGTGAAAAAGGACCTGATCATCAAGCT
GCCTAAGTACTCCCTGTTCGAGCTGGAAAACGGCCGGAAGAGAATGCTGGCCT
CTGCCGGCGAACTGCAGAAGGGAAACGAACTGGCCCTGCCCTCCAAATATGTG
AACTTCCTGTACCTGGCCAGCCACTATGAGAAGCTGAAGGGCTCCCCCGAGGA
TAATGAGCAGAAACAGCTGTTTGTGGAACAGCACAAACACTACCTGGACGAG
ATCATCGAGCAGATCAGCGAGTTCTCCAAGAGAGTGATCCTGGCCGACGCTAA
TCTGGACAAGGTGCTGAGCGCCTACAACAAGCACAGAGACAAGCCTATCAGA
GAGCAGGCCGAGAATATCATCCACCTGTTTACCCTGACCAATCTGGGAGCCCC
TGCCGCCTTCAAGTACTTTGACACCACCATCGACCGGAAGAGGTACACCAGCA
CCAAAGAGGTGCTGGACGCCACCCTGATCCACCAGAGCATCACCGGCCTGTAC
GAGACACGGATCGACCTGTCTCAGCTGGGAGGCGACGCCTATCCCTATGACGT
GCCCGATTATGCCAGCCTGGGCAGCGGCTCCCCCAAGAAAAAACGCAAGGTG
GAAG
SEQ ID NO: 7-DNA Sequence of the Triple Repressor BFP CRISPRi System
GATCCAGCGAGCTGATTAAGGAGAACATGCACATGAAGCTGTACATGGAGGG
CACCGTGGACAACCATCACTTCAAGTGCACATCCGAGGGCGAAGGCAAGCCCT
ACGAGGGCACCCAGACCATGAGAATCAAGGTGGTCGAGGGCGGCCCTCTCCCC
TTCGCCTTCGACATCCTGGCTACTAGCTTCCTCTACGGCAGCAAGACCTTCATC
AACCACACCCAGGGCATCCCCGACTTCTTCAAGCAGTCCTTCCCTGAGGGCTTC
ACATGGGAGAGAGTCACCACATACGAAGACGGGGGCGTGCTGACCGCTACCC
AGGACACCAGCCTCCAGGACGGCTGCCTCATCTACAACGTCAAGATCAGAGGG
GTGAACTTCACATCCAACGGCCCTGTGATGCAGAAGAAAACACTCGGCTGGGA
GGCCTTCACCGAGACGCTGTACCCCGCTGACGGCGGCCTGGAAGGCAGAAACG
ACATGGCCCTGAAGCTCGTGGGCGGGAGCCATCTGATCGCAAACATCAAGACC
ACATATAGATCCAAGAAACCCGCTAAGAACCTCAAGATGCCTGGCGTCTACTA
TGTGGACTACAGACTGGAAAGAATCAAGGAGGCCAACAACGAGACCTACGTC
GAGCAGCACGAGGTGGCAGTGGCCAGATACTGCGACCTCCCTAGCAAACTGG
GGCACAAGCTTAATGGATCCAGTAAACGACCTGCCGCCACTAAAAAAGCCGG
ACAGGCTAAGAAGAAGAAAGGAGGTTCAGGAGGATCTGGGGGGAGCGGAGG
GAGCATGTCCAGGCGGAAACAGAGCAACCCCCGGCAGATCAAGCGTTCCCTCG
GAGACATGGAGGCCAGAGAGGAGGTGCAGTTGGTGGGTGCCAGCCACATGGA
GCAAAAGGCCACGGCACCTGAAGCCCCGAGCCCTGGCTCCGGCGCAACAAAC
TTCTCTCTGCTGAAACAAGCCGGAGATGTCGAAGAGAATCCTGGACCGATGGC
CAAGCCTTTGTCTCAAGAAGAATCCACCCTCATTGAAAGAGCAACGGCTACAA
TCAACAGCATCCCCATCTCTGAAGACTACAGCGTCGCCAGCGCAGCTCTCTCTA
GCGACGGCCGCATCTTCACTGGTGTCAATGTATATCATTTTACTGGGGGACCTT
GTGCAGAACTCGTGGTGCTGGGCACTGCTGCTGCTGCGGCAGCTGGCAACCTG
ACTTGTATCGTCGCGATCGGAAATGAGAACAGGGGCATCTTGAGCCCCTGCGG
ACGGTGCCGACAGGTGCTTCTCGATCTGCATCCTGGGATCAAAGCCATAGTGA
AGGACAGTGATGGACAGCCGACGGCAGTTGGGATTCGTGAATTGCTGCCCTCT
GGTTATGTGTGGGAGGGCTAAGAATTCAATCAACCTCTGGATTACAAAATTTG
TGAAAGATTGACTGGTATTCTTAACTATGTTGCTCCTTTTACGCTATGTGGATA
CGCTGCTTTAATGCCTTTGTATCATGCTATTGCTTCCCGTATGGCTTTCATTTTC
TCCTCCTTGTATAAATCCTGGTTGCTGTCTCTTTATGAGGAGTTGTGGCCCGTTG
TCAGGCAACGTGGCGTGGTGTGCACTGTGTTTGCTGACGCAACCCCCACTGGTT
GGGGCATTGCCACCACCTGTCAGCTCCTTTCCGGGACTTTCGCTTTCCCCCTCC
CTATTGCCACGGCGGAACTCATCGCCGCCTGCCTTGCCCGCTGCTGGACAGGG
GCTCGGCTGTTGGGCACTGACAATTCCGTGGTGTTGTCGGGGAAGCTGACGTC
CTTTCCATGGCTGCTCGCCTGTGTTGCCACCTGGATTCTGCGCGGGACGTCCTT
CTGCTACGTCCCTTCGGCCCTCAATCCAGCGGACCTTCCTTCCCGCGGCCTGCT
GCCGGCTCTGCGGCCTCTTCCGCGTCTTCGCCTTCGCCCTCAGACGAGTCGGAT
CTCCCTTTGGGCCGCCTCCCCGCGAGACGTTTCATTTCCGTCTCTGGTACCACTT
TTTAAAAGAAAAGGGGGGACTGGAAGGGCTAATTCACTCCCAACGAAGTCAA
GATATCCTTGATCTGTGGATCGTTAACTACCACACACAAGGCTACTTCCCTGAT
TGGCAGAACTACACACCAGGGCCAGGGATCAAATATCCACTGACCTTTGGATG
GTGCTACAAGCTAGTACCAGTTGAGCAAGAGAAGGTAGAAGAAGCCAATGAA
GGAGAGAACACCCGCTTGTTACACCCTGTGAGCCTGCATGGGATGGATGACCC
GGAGAGAGAAGTATTAGAGTGGAGGGTTAACTTAATTAAGACAGCCGCCTAG
CATTTCATCACATGGCCCGAGAGCTGCATCCGGACTGTACTGGCTCTCTCTGGT
TAGACCAGATCTGAGCCTGGGAGCTCTCTGGCTAACTAGGGAACCCACTGCTT
AAGCCTCAATAAAGCTTGCCTTGAGTGCTTCAAGTAGTGTGTGCCCGTCTGTTG
TGTGACTCTGGTAACTAGAGATCCCTCAGACCCTTTTAGTCAGTGTGGAAAATC
TCTAGCAGGGCCCTCTAGAGTTTAAACCCGCTGATCAGCCTCGACTGTGCCTTC
TAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTG
TCTGAGTAGGTGTCATTCTATTCTGGGGGGTGGGGTGGGGCAGGACAGCAAGG
GGGAGGATTGGGAAGAGAATAGCAGGCATGCTGGGGATGCGGTGGGCTCTAT
GGCTTCTGAGGCGGAAAGAACCAGCTGGGGCTCTAGGGGGTATCCCCACGCGC
CCTGTAGCGGCGCATTAAGCGCGGCGGGTGTGGTGGTTACGCGCAGCGTGACC
GCTACACTTGCCAGCGCCCTAGCGCCCGCTCCTTTCGCTTTCTTCCCTTCCTTTC
TCGCCACGTTCGCCGGCTTTCCCCGTCAAGCTCTAAATCGGGGGCTCCCTTTAG
GGTTCCGATTTAGTGCTTTACGGCACCTCGACCCCAAAAAACTTGATTAGGGTG
ATGGTTCACGTAGTGGGCCATCGCCCTGATAGACGGTTTTTCGCCCTTTGACGT
TGGAGTCCACGTTCTTTAATAGTGGACTCTTGTTCCAAACTGGAACAACACTCA
ACCCTATCTCGGTCTATTCTTTTGATTTATAAGGGATTTTGCCGATTTCGGCCTA
TTGGTTAAAAAATGAGCTGATTTAACAAAAATTTAACGCGAATTAATTCTGTG
GAATGTGTGTCAGTTAGGGTGTGGAAAGTCCCCAGGCTCCCCAGCAGGCAGAA
GTATGCAAAGCATGCATCTCAATTAGTCAGCAACCAGGTGTGGAAAGTCCCCA
GGCTCCCCAGCAGGCAGAAGTATGCAAAGCATGCATCTCAATTAGTCAGCAAC
CATAGTCCCGCCCCTAACTCCGCCCATCCCGCCCCTAACTCCGCCCAGTTCCGC
CCATTCTCCGCCCCATGGCTGACTAATTTTTTTTATTTATGCAGAGGCCGAGGC
CGCCTCTGCCTCTGAGCTATTCCAGAAGTAGTGAGGAGGCTTTTTTGGAGGCCT
AGGCTTTTGCAAAAAGCTCCCGGGAGCTTGTATATCCATTTTCGGATCTGATCA
GCACGTGTTGACAATTAATCATCGGCATAGTATATCGGCATAGTATAATACGA
CAAGGTGAGGAACTAAACCATGGCCAAGTTGACCAGTGCCGTTCCGGTGCTCA
CCGCGCGCGACGTCGCCGGAGCGGTCGAGTTCTGGACCGACCGGCTCGGGTTC
TCCCGGGACTTCGTGGAGGACGACTTCGCCGGTGTGGTCCGGGACGACGTGAC
CCTGTTCATCAGCGCGGTCCAGGACCAGGTGGTGCCGGACAACACCCTGGCCT
GGGTGTGGGTGCGCGGCCTGGACGAGCTGTACGCCGAGTGGTCGGAGGTCGTG
TCCACGAACTTCCGGGACGCCTCCGGGCCGGCCATGACCGAGATCGGCGAGCA
GCCGTGGGGGCGGGAGTTCGCCCTGCGCGACCCGGCCGGCAACTGCGTGCACT
TCGTGGCCGAGGAGCAGGACTGACACGTGCTACGAGATTTCGATTCCACCGCC
GCCTTCTATGAAAGGTTGGGCTTCGGAATCGTTTTCCGGGACGCCGGCTGGAT
GATCCTCCAGCGCGGGGATCTCATGCTGGAGTTCTTCGCCCACCCCAACTTGTT
TATTGCAGCTTATAATGGTTACAAATAAAGCAATAGCATCACAAATTTCACAA
ATAAAGCATTTTTTTCACTGCATTCTAGTTGTGGTTTGTCCAAACTCATCAATGT
ATCTTATCATGTCTGTATACCGTCGACCTCTAGCTAGAGCTTGGCGTAATCATG
GTCATAGCTGTTTCCTGTGTGAAATTGTTATCCGCTCACAATTCCACACAACAT
ACGAGCCGGAAGCATAAAGTGTAAAGCCTGGGGTGCCTAATGAGTGAGCTAA
CTCACATTAATTGCGTTGCGCTCACTGCCCGCTTTCCAGTCGGGAAACCTGTCG
TGCCAGCTGCATTAATGAATCGGCCAACGCGCGGGGAGAGGCGGTTTGCGTAT
TGGGCGCTCTTCCGCTTCCTCGCTCACTGACTCGCTGCGCTCGGTCGTTCGGCT
GCGGCGAGCGGTATCAGCTCACTCAAAGGCGGTAATACGGTTATCCACAGAAT
CAGGGGATAACGCAGGAAAGAACATGTGAGCAAAAGGCCAGCAAAAGGCCA
GGAACCGTAAAAAGGCCGCGTTGCTGGCGTTTTTCCATAGGCTCCGCCCCCCT
GACGAGCATCACAAAAATCGACGCTCAAGTCAGAGGTGGCGAAACCCGACAG
GACTATAAAGATACCAGGCGTTTCCCCCTGGAAGCTCCCTCGTGCGCTCTCCTG
TTCCGACCCTGCCGCTTACCGGATACCTGTCCGCCTTTCTCCCTTCGGGAAGCG
TGGCGCTTTCTCATAGCTCACGCTGTAGGTATCTCAGTTCGGTGTAGGTCGTTC
GCTCCAAGCTGGGCTGTGTGCACGAACCCCCCGTTCAGCCCGACCGCTGCGCC
TTATCCGGTAACTATCGTCTTGAGTCCAACCCGGTAAGACACGACTTATCGCCA
CTGGCAGCAGCCACTGGTAACAGGATTAGCAGAGCGAGGTATGTAGGCGGTG
CTACAGAGTTCTTGAAGTGGTGGCCTAACTACGGCTACACTAGAAGAACAGTA
TTTGGTATCTGCGCTCTGCTGAAGCCAGTTACCTTCGGAAAAAGAGTTGGTAGC
TCTTGATCCGGCAAACAAACCACCGCTGGTAGCGGTGGTTTTTTTGTTTGCAAG
CAGCAGATTACGCGCAGAAAAAAAGGATCTCAAGAAGATCCTTTGATCTTTTC
TACGGGGTCTGACGCTCAGTGGAACGAAAACTCACGTTAAGGGATTTTGGTCA
TGAGATTATCAAAAAGGATCTTCACCTAGATCCTTTTAAATTAAAAATGAAGTT
TTAAATCAATCTAAAGTATATATGAGTAAACTTGGTCTGACAGTTACCAATGCT
TAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGC
CCCAGTGCTGCAATGATACCGCGGGACCCACGCTCACCGGCTCCAGATTTATC
AGCAATAAACCAGCCAGCCGGAAGGGCCGAGCGCAGAAGTGGTCCTGCAACT
TTATCCGCCTCCATCCAGTCTATTAATTGTTGCCGGGAAGCTAGAGTAAGTAGT
TCGCCAGTTAATAGTTTGCGCAACGTTGTTGCCATTGCTACAGGCATCGTGGTG
TCACGCTCGTCGTTTGGTATGGCTTCATTCAGCTCCGGTTCCCAACGATCAAGG
CGAGTTACATGATCCCCCATGTTGTGCAAAAAAGCGGTTAGCTCCTTCGGTCCT
CCGATCGTTGTCAGAAGTAAGTTGGCCGCAGTGTTATCACTCATGGTTATGGCA
GCACTGCATAATTCTCTTACTGTCATGCCATCCGTAAGATGCTTTTCTGTGACT
GGTGAGTACTCAACCAAGTCATTCTGAGAATAGTGTATGCGGCGACCGAGTTG
CTCTTGCCCGGCGTCAATACGGGATAATACCGCGCCACATAGCAGAACTTTAA
AAGTGCTCATCATTGGAAAACGTTCTTCGGGGCGAAAACTCTCAAGGATCTTA
CCGCTGTTGAGATCCAGTTCGATGTAACCCACTCGTGCACCCAACTGATCTTCA
GCATCTTTTACTTTCACCAGCGTTTCTGGGTGAGCAAAAACAGGAAGGCAAAA
TGCCGCAAAAAAGGGAATAAGGGCGACACGGAAATGTTGAATACTCATACTCT
TCCTTTTTCAATATTATTGAAGCATTTATCAGGGTTATTGTCTCATGAGCGGAT
ACATATTTGAATGTATTTAGAAAAATAAACAAATAGGGGTTCCGCGCACATTT
CCCCGAAAAGTGCCACCTGACGTCGACGGATCGGGAGATCTCCCGATCCCCTA
TGGTGCACTCTCAGTACAATCTGCTCTGATGCCGCATAGTTAAGCCAGTATCTG
CTCCCTGCTTGTGTGTTGGAGGTCGCTGAGTAGTGCGCGAGCAAAATTTAAGCT
ACAACAAGGCAAGGCTTGACCGACAATTGCATGAAGAATCTGCTTAGGGTTAG
GCGTTTTGCGCTGCTTCGCGATGTACGGGCCAGATATACGCGTTGACATTGATT
ATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGC
CCAACGACCCCCGCCCATTGACGTCAATAATGACGTATGTTCCCATAGTAACG
CCAATAGGGACTTTCCATTGACGTCAATGGGTGGAGTATTTACGGTAAACTGC
CCACTTGGCAGTACATCAAGTGTATCATATGCCAAGTACGCCCCCTATTGACGT
CAATGACGGTAAATGGCCCGCCTGGCATTATGCCCAGTACATGACCTTATGGG
ACTTTCCTACTTGGCAGTACATCTACGTATTAGTCATCGCTATTACCATGGTGA
TGCGGTTTTGGCAGTACATCAATGGGCGTGGATAGCGGTTTGACTCACGGGGA
TTTCCAAGTCTCCACCCCATTGACGTCAATGGGAGTTTGTTTTGGCACCAAAAT
CAACGGGACTTTCCAAAATGTCGTAACAACTCCGCCCCATTGACGCAAATGGG
CGGTAGGCGTGTACGGTGGGAGGTCTATATAAGCAGCGCGTTTTGCCTGTACT
GGCTCTCTCTGGTTAGACCAGATCTGAGCCTGGGAGCTCTCTGGCTAACTAGG
GAACCCACTGCTTAAGCCTCAATAAAGCTTGCCTTGAGTGCTTCAAGTAGTGTG
TGCCCGTCTGTTGTGTGACTCTGGTAACTAGAGATCCCTCAGACCCTTTTAGTC
AGTGTGGAAAATCTCTAGCAGTGGCGCCCGAACAGGGACTTGAAAGCGAAAG
GGAAACCAGAGGAGCTCTCTCGACGCAGGACTCGGCTTGCTGAAGCGCGCACG
GCAAGAGGCGAGGGGCGGCGACTGGTGAGTACGCCAAAAATTTTGACTAGCG
GAGGCTAGAAGGAGAGAGATGGGTGCGAGAGCGTCAGTATTAAGCGGGGGAG
AATTAGATCGCGATGGGAAAAAATTCGGTTAAGGCCAGGGGGAAAGAAAAAA
TATAAATTAAAACATATAGTATGGGCAAGCAGGGAGCTAGAACGATTCGCAGT
TAATCCTGGCCTGTTAGAAACATCAGAAGGCTGTAGACAAATACTGGGACAGC
TACAACCATCCCTTCAGACAGGATCAGAAGAACTTAGATCATTATATAATACA
GTAGCAACCCTCTATTGTGTGCATCAAAGGATAGAGATAAAAGACACCAAGGA
AGCTTTAGACAAGATAGAGGAAGAGCAAAACAAAAGTAAGACCACCGCACAG
CAAGCGGCCGCTGATCTTCAGACCTGGAGGAGGAGATATGAGGGACAATTGG
AGAAGTGAATTATATAAATATAAAGTAGTAAAAATTGAACCATTAGGAGTAGC
ACCCACCAAGGCAAAGAGAAGAGTGGTGCAGAGAGAAAAAAGAGCAGTGGG
AATAGGAGCTTTGTTCCTTGGGTTCTTGGGAGCAGCAGGAAGCACTATGGGCG
CAGCGTCAATGACGCTGACGGTACAGGCCAGACAATTATTGTCTGGTATAGTG
CAGCAGCAGAACAATTTGCTGAGGGCTATTGAGGCGCAACAGCATCTGTTGCA
ACTCACAGTCTGGGGCATCAAGCAGCTCCAGGCAAGAATCCTGGCTGTGGAAA
GATACCTAAAGGATCAACAGCTCCTGGGGATTTGGGGTTGCTCTGGAAAACTC
ATTTGCACCACTGCTGTGCCTTGGAATGCTAGTTGGAGTAATAAATCTCTGGAA
CAGATTTGGAATCACACGACCTGGATGGAGTGGGACAGAGAAATTAACAATTA
CACAAGCTTAATACACTCCTTAATTGAAGAATCGCAAAACCAGCAAGAAAAGA
ATGAACAAGAATTATTGGAATTAGATAAATGGGCAAGTTTGTGGAATTGGTTT
AACATAACAAATTGGCTGTGGTATATAAAATTATTCATAATGATAGTAGGAGG
CTTGGTAGGTTTAAGAATAGTTTTTGCTGTACTTTCTATAGTGAATAGAGTTAG
GCAGGGATATTCACCATTATCGTTTCAGACCCACCTCCCAACCCCGAGGGGAC
CCGACAGGCCCGAAGGAATAGAAGAAGAAGGTGGAGAGAGAGACAGAGACA
GATCCATTCGATTAGTGAACGGATCGGCACTGCGTGCGCCAATTCTGCAGACA
AATGGCAGTATTCATCCACAATTTTAAAAGAAAAGGGGGGATTGGGGGGTACA
GTGCAGGGGAAAGAATAGTAGACATAATAGCAACAGACATACAAACTAAAGA
ATTACAAAAACAAATTACAAAAATTCAAAATTTTCGGGTTTATTACAGGGACA
GCAGAGATCCAGTTTGGTTAATTAGGGTTAATTAGCTAGCGGCTCCGGTGCCC
GTCAGTGGGCAGAGCGCACATCGCCCACAGTCCCCGAGAAGTTGGGGGGAGG
GGTCGGCAATTGATCCGGTGCCTAGAGAAGGTGGCGCGGGGTAAACTGGGAA
AGTGATGTCGTGTACTGGCTCCGCCTTTTTCCCGAGGGTGGGGGAGAACCGTA
TATAAGTGCAGTAGTCGCCGTGAACGTTCTTTTTCGCAACGGGTTTGCCGCCAG
AACACAGGCTCGAGGCCACCATGAACAATTCCCAGGGAAGAGTGACCTTCGA
GGATGTCACTGTGAACTTCACCCAGGGGGAGTGGCAGCGGCTGAATCCCGAAC
AGAGAAACTTGTACAGGGATGTGATGCTGGAGAATTACAGCAACCTTGTCTCT
GTGGGACAAGGGGAAACCACCAAACCCGATGTGATCTTGAGGTTGGAACAAG
GAAAGGAGCCATGGTTGGAGGAAGAGGAAGTGCTGGGAAGTGGCCGTGCAGA
AAAAAATGGGGACATTGGAGGGCAGATTTGGAAGCCAAAGGATGTGAAAGAG
AGTCTCGGTGGGTCTGGCGGTTCTGTGCAGGTGAAAAGGGTGCTGGAAAAATC
CCCCGGCAAACTCCTCGTGAAGATGCCCTTCCAGGCTTCCCCTGGCGGAAAAG
GTGAAGGGGGTGGCGCAACCACATCTGCCCAGGTCATGGTCATCAAGCGACCT
GGAAGGAAAAGAAAGGCCGAGGCTGACCCTCAGGCCATTCCAAAGAAACGGG
GACGCAAGCCAGGGTCCGTGGTCGCAGCTGCAGCAGCTGAGGCTAAGAAAAA
GGCAGTGAAGGAAAGCTCCATCCGCAGTGTGCAGGAGACTGTCCTGCCCATCA
AGAAGAGGAAGACTAGGGAGACCGTGTCCATCGAGGTCAAAGAAGTGGTCAA
GCCCCTGCTCGTGTCCACCCTGGGCGAAAAATCTGGAAAGGGGCTCAAAACAT
GCAAGTCACCTGGACGGAAAAGCAAGGAGTCTAGTCCAAAGGGGCGCTCAAG
CTCCGCTTCTAGTCCCCCTAAAAAGGAACACCATCACCATCACCATCACGCCG
AGTCTCCTAAGGCTCCTATGCCACTGCTCCCACCACCTCCACCACCTGAGCCAC
AGTCAAGCGAAGACCCCATCAGCCCACCCGAGCCTCAGGATCTGTCCTCTAGT
ATTTGCAAAGAGGAAAAGATGCCCAGAGCAGGCAGCCTGGAGAGTGATGGCT
GTCCAAAAGAACCCGCCAAGACCCAGCCTATGGTGGCAGCCGCTGCAACTACC
ACCACAACCACAACTACCACAGTGGCCGAAAAATACAAGCATCGCGGCGAGG
GCGAACGAAAGGACATTGTGTCAAGCTCCATGCCCAGACCTAACCGGGAGGA
ACCAGTCGATAGTAGGACACCCGTGACTGAGAGAGTCTCAGGCTCCGCCGGCA
GCGCTGCCGGCTCAGGGGAGTTTCCTAAGAAAAAGCGGAAAGTGCGTACGGA
CAAGAAGTACAGCATCGGCCTGGCCATCGGCACCAACTCTGTGGGCTGGGCCG
TGATCACCGACGAGTACAAGGTGCCCAGCAAGAAATTCAAGGTGCTGGGCAA
CACCGACCGGCACAGCATCAAGAAGAACCTGATCGGCGCCCTGCTGTTCGACA
GCGGAGAAACAGCCGAGGCCACCCGGCTGAAGAGAACCGCCAGAAGAAGATA
CACCAGACGGAAGAACCGGATCTGCTATCTGCAAGAGATCTTCAGCAACGAGA
TGGCCAAGGTGGACGACAGCTTCTTCCACAGACTGGAAGAGTCCTTCCTGGTG
GAAGAGGATAAGAAGCACGAGCGGCACCCCATCTTCGGCAACATCGTGGACG
AGGTGGCCTACCACGAGAAGTACCCCACCATCTACCACCTGAGAAAGAAACTG
GTGGACAGCACCGACAAGGCCGACCTGCGGCTGATCTATCTGGCCCTGGCCCA
CATGATCAAGTTCCGGGGCCACTTCCTGATCGAGGGCGACCTGAACCCCGACA
ACAGCGACGTGGACAAGCTGTTCATCCAGCTGGTGCAGACCTACAACCAGCTG
TTCGAGGAAAACCCCATCAACGCCAGCGGCGTGGACGCCAAGGCCATCCTGTC
TGCCAGACTGAGCAAGAGCAGACGGCTGGAAAATCTGATCGCCCAGCTGCCCG
GCGAGAAGAAGAATGGCCTGTTCGGCAACCTGATTGCCCTGAGCCTGGGCCTG
ACCCCCAACTTCAAGAGCAACTTCGACCTGGCCGAGGATGCCAAACTGCAGCT
GAGCAAGGACACCTACGACGACGACCTGGACAACCTGCTGGCCCAGATCGGC
GACCAGTACGCCGACCTGTTTCTGGCCGCCAAGAACCTGTCCGACGCCATCCT
GCTGAGCGACATCCTGAGAGTGAACACCGAGATCACCAAGGCCCCCCTGAGCG
CCTCTATGATCAAGAGATACGACGAGCACCACCAGGACCTGACCCTGCTGAAA
GCTCTCGTGCGGCAGCAGCTGCCTGAGAAGTACAAAGAGATTTTCTTCGACCA
GAGCAAGAACGGCTACGCCGGCTACATCGATGGCGGAGCCAGCCAGGAAGAG
TTCTACAAGTTCATCAAGCCCATCCTGGAAAAGATGGACGGCACCGAGGAACT
GCTCGTGAAGCTGAACAGAGAGGACCTGCTGCGGAAGCAGCGGACCTTCGAC
AACGGCAGCATCCCCCACCAGATCCACCTGGGAGAGCTGCACGCCATTCTGCG
GCGGCAGGAAGATTTTTACCCATTCCTGAAGGACAACCGGGAAAAGATCGAG
AAGATCCTGACCTTCCGCATCCCCTACTACGTGGGCCCTCTGGCCAGGGGAAA
CAGCAGATTCGCCTGGATGACCAGAAAGAGCGAGGAAACCATCACCCCCTGG
AACTTCGAGGAAGTGGTGGACAAGGGCGCCAGCGCCCAGAGCTTCATCGAGC
GGATGACCAACTTCGATAAGAACCTGCCCAACGAGAAGGTGCTGCCCAAGCAC
AGCCTGCTGTACGAGTACTTCACCGTGTACAACGAGCTGACCAAAGTGAAATA
CGTGACCGAGGGAATGAGAAAGCCCGCCTTCCTGAGCGGCGAGCAGAAAAAA
GCCATCGTGGACCTGCTGTTCAAGACCAACCGGAAAGTGACCGTGAAGCAGCT
GAAAGAGGACTACTTCAAGAAAATCGAGTGCTTCGACTCCGTGGAAATCTCCG
GCGTGGAAGATCGGTTCAACGCCTCCCTGGGCACATACCACGATCTGCTGAAA
ATTATCAAGGACAAGGACTTCCTGGACAATGAGGAAAACGAGGACATTCTGG
AAGATATCGTGCTGACCCTGACACTGTTTGAGGACAGAGAGATGATCGAGGAA
CGGCTGAAAACCTATGCCCACCTGTTCGACGACAAAGTGATGAAGCAGCTGAA
GCGGCGGAGATACACCGGCTGGGGCAGGCTGAGCCGGAAGCTGATCAACGGC
ATCCGGGACAAGCAGTCCGGCAAGACAATCCTGGATTTCCTGAAGTCCGACGG
CTTCGCCAACAGAAACTTCATGCAGCTGATCCACGACGACAGCCTGACCTTTA
AAGAGGACATCCAGAAAGCCCAGGTGTCCGGCCAGGGCGATAGCCTGCACGA
GCACATTGCCAATCTGGCCGGCAGCCCCGCCATTAAGAAGGGCATCCTGCAGA
CAGTGAAGGTGGTGGACGAGCTCGTGAAAGTGATGGGCCGGCACAAGCCCGA
GAACATCGTGATCGAAATGGCCAGAGAGAACCAGACCACCCAGAAGGGACAG
AAGAACAGCCGCGAGAGAATGAAGCGGATCGAAGAGGGCATCAAAGAGCTGG
GCAGCCAGATCCTGAAAGAACACCCCGTGGAAAACACCCAGCTGCAGAACGA
GAAGCTGTACCTGTACTACCTGCAGAATGGGGGGATATGTACGTGGACCAGG
AACTGGACATCAACCGGCTGTCCGACTACGATGTGGACGCTATCGTGCCTCAG
AGCTTTCTGAAGGACGACTCCATCGATAACAAAGTGCTGACTCGGAGCGACAA
GAACCGGGGCAAGAGCGACAACGTGCCCTCCGAAGAGGTCGTGAAGAAGATG
AAGAACTACTGGCGCCAGCTGCTGAATGCCAAGCTGATTACCCAGAGGAAGTT
CGACAATCTGACCAAGGCCGAGAGAGGCGGCCTGAGCGAACTGGATAAGGCC
GGCTTCATCAAGAGACAGCTGGTGGAAACCCGGCAGATCACAAAGCACGTGG
CACAGATCCTGGACTCCCGGATGAACACTAAGTACGACGAGAACGACAAACT
GATCCGGGAAGTGAAAGTGATCACCCTGAAGTCCAAGCTGGTGTCCGATTTCC
GGAAGGATTTCCAGTTTTACAAAGTGCGCGAGATCAACAACTACCACCACGCC
CACGACGCCTACCTGAACGCCGTCGTGGGAACCGCCCTGATCAAAAAGTACCC
TAAGCTGGAAAGCGAGTTCGTGTACGGCGACTACAAGGTGTACGACGTGCGGA
AGATGATCGCCAAGAGCGAGCAGGAAATCGGCAAGGCTACCGCCAAGTACTT
CTTCTACAGCAACATCATGAACTTTTTCAAGACCGAGATTACCCTGGCCAACG
GCGAGATCCGGAAGCGGCCTCTGATCGAGACAAACGGCGAAACAGGCGAGAT
CGTGTGGGATAAGGGCCGGGACTTTGCCACCGTGCGGAAAGTGCTGTCTATGC
CCCAAGTGAATATCGTGAAAAAGACCGAGGTGCAGACAGGCGGCTTCAGCAA
AGAGTCTATCCTGCCCAAGAGGAACAGCGACAAGCTGATCGCCAGAAAGAAG
GACTGGGACCCTAAGAAGTACGGCGGCTTCGACAGCCCCACCGTGGCCTATTC
TGTGCTGGTGGTGGCCAAAGTGGAAAAGGGCAAGTCCAAGAAACTGAAGAGT
GTGAAAGAGCTGCTGGGGATCACCATCATGGAAAGAAGCAGCTTCGAGAAGA
ATCCCATCGACTTTCTGGAAGCCAAGGGCTACAAAGAAGTGAAAAAGGACCTG
ATCATCAAGCTGCCTAAGTACTCCCTGTTCGAGCTGGAAAACGGCCGGAAGAG
AATGCTGGCCTCTGCCGGCGAACTGCAGAAGGGAAACGAACTGGCCCTGCCCT
CCAAATATGTGAACTTCCTGTACCTGGCCAGCCACTATGAGAAGCTGAAGGGC
TCCCCCGAGGATAATGAGCAGAAACAGCTGTTTGTGGAACAGCACAAACACTA
CCTGGACGAGATCATCGAGCAGATCAGCGAGTTCTCCAAGAGAGTGATCCTGG
CCGACGCTAATCTGGACAAGGTGCTGAGCGCCTACAACAAGCACAGAGACAA
GCCTATCAGAGAGCAGGCCGAGAATATCATCCACCTGTTTACCCTGACCAATC
TGGGAGCCCCTGCCGCCTTCAAGTACTTTGACACCACCATCGACCGGAAGAGG
TACACCAGCACCAAAGAGGTGCTGGACGCCACCCTGATCCACCAGAGCATCAC
CGGCCTGTACGAGACACGGATCGACCTGTCTCAGCTGGGAGGCGACGCCTATC
CCTATGACGTGCCCGATTATGCCAGCCTGGGCAGCGGCTCCCCCAAGAAAAAA
CGCAAGGTGGAAG
SEQ ID NO: 8-DNA Sequence of ZIM3 KRAB
AACAATTCCCAGGGAAGAGTGACCTTCGAGGATGTCACTGTGAACTTCACCCA
GGGGGAGTGGCAGCGGCTGAATCCCGAACAGAGAAACTTGTACAGGGATGTG
ATGCTGGAGAATTACAGCAACCTTGTCTCTGTGGGACAAGGGGAAACCACCAA
ACCCGATGTGATCTTGAGGTTGGAACAAGGAAAGGAGCCATGGTTGGAGGAA
GAGGAAGTGCTGGGAAGTGGCCGTGCAGAAAAAAATGGGGACATTGGAGGGC
AGATTTGGAAGCCAAAGGATGTGAAAGAGAGTCTC
SEQ ID NO: 9-DNA Sequence of MeCP2
GTGCAGGTGAAAAGGGTGCTGGAAAAATCCCCCGGCAAACTCCTCGTGAAGAT
GCCCTTCCAGGCTTCCCCTGGCGGAAAAGGTGAAGGGGGTGGCGCAACCACAT
CTGCCCAGGTCATGGTCATCAAGCGACCTGGAAGGAAAAGAAAGGCCGAGGC
TGACCCTCAGGCCATTCCAAAGAAACGGGGACGCAAGCCAGGGTCCGTGGTCG
CAGCTGCAGCAGCTGAGGCTAAGAAAAAGGCAGTGAAGGAAAGCTCCATCCG
CAGTGTGCAGGAGACTGTCCTGCCCATCAAGAAGAGGAAGACTAGGGAGACC
GTGTCCATCGAGGTCAAAGAAGTGGTCAAGCCCCTGCTCGTGTCCACCCTGGG
CGAAAAATCTGGAAAGGGGCTCAAAACATGCAAGTCACCTGGACGGAAAAGC
AAGGAGTCTAGTCCAAAGGGGCGCTCAAGCTCCGCTTCTAGTCCCCCTAAAAA
GGAACACCATCACCATCACCATCACGCCGAGTCTCCTAAGGCTCCTATGCCAC
TGCTCCCACCACCTCCACCACCTGAGCCACAGTCAAGCGAAGACCCCATCAGC
CCACCCGAGCCTCAGGATCTGTCCTCTAGTATTTGCAAAGAGGAAAAGATGCC
CAGAGCAGGCAGCCTGGAGAGTGATGGCTGTCCAAAAGAACCCGCCAAGACC
CAGCCTATGGTGGCAGCCGCTGCAACTACCACCACAACCACAACTACCACAGT
GGCCGAAAAATACAAGCATCGCGGCGAGGGCGAACGAAAGGACATTGTGTCA
AGCTCCATGCCCAGACCTAACCGGGAGGAACCAGTCGATAGTAGGACACCC
GTGACTGAGAGAGTCTCA
SEQ ID NO: 10-DNA Sequence of dCas9 (Staphylococcus pyogenes)
GACAAGAAGTACAGCATCGGCCTGGCCATCGGCACCAACTCTGTGGGCTGGGC
CGTGATCACCGACGAGTACAAGGTGCCCAGCAAGAAATTCAAGGTGCTGGGC
AACACCGACCGGCACAGCATCAAGAAGAACCTGATCGGCGCCCTGCTGTTCGA
CAGCGGAGAAACAGCCGAGGCCACCCGGCTGAAGAGAACCGCCAGAAGAAGA
TACACCAGACGGAAGAACCGGATCTGCTATCTGCAAGAGATCTTCAGCAACGA
GATGGCCAAGGTGGACGACAGCTTCTTCCACAGACTGGAAGAGTCCTTCCTGG
TGGAAGAGGATAAGAAGCACGAGCGGCACCCCATCTTCGGCAACATCGTGGA
CGAGGTGGCCTACCACGAGAAGTACCCCACCATCTACCACCTGAGAAAGAAAC
TGGTGGACAGCACCGACAAGGCCGACCTGCGGCTGATCTATCTGGCCCTGGCC
CACATGATCAAGTTCCGGGGCCACTTCCTGATCGAGGGCGACCTGAACCCCGA
CAACAGCGACGTGGACAAGCTGTTCATCCAGCTGGTGCAGACCTACAACCAGC
TGTTCGAGGAAAACCCCATCAACGCCAGCGGCGTGGACGCCAAGGCCATCCTG
TCTGCCAGACTGAGCAAGAGCAGACGGCTGGAAAATCTGATCGCCCAGCTGCC
CGGCGAGAAGAAGAATGGCCTGTTCGGCAACCTGATTGCCCTGAGCCTGGGCC
TGACCCCCAACTTCAAGAGCAACTTCGACCTGGCCGAGGATGCCAAACTGCAG
CTGAGCAAGGACACCTACGACGACGACCTGGACAACCTGCTGGCCCAGATCGG
CGACCAGTACGCCGACCTGTTTCTGGCCGCCAAGAACCTGTCCGACGCCATCC
TGCTGAGCGACATCCTGAGAGTGAACACCGAGATCACCAAGGCCCCCCTGAGC
GCCTCTATGATCAAGAGATACGACGAGCACCACCAGGACCTGACCCTGCTGAA
AGCTCTCGTGCGGCAGCAGCTGCCTGAGAAGTACAAAGAGATTTTCTTCGACC
AGAGCAAGAACGGCTACGCCGGCTACATCGATGGCGGAGCCAGCCAGGAAGA
GTTCTACAAGTTCATCAAGCCCATCCTGGAAAAGATGGACGGCACCGAGGAAC
TGCTCGTGAAGCTGAACAGAGAGGACCTGCTGCGGAAGCAGCGGACCTTCGAC
AACGGCAGCATCCCCCACCAGATCCACCTGGGAGAGCTGCACGCCATTCTGCG
GCGGCAGGAAGATTTTTACCCATTCCTGAAGGACAACCGGGAAAAGATCGAG
AAGATCCTGACCTTCCGCATCCCCTACTACGTGGGCCCTCTGGCCAGGGGAAA
CAGCAGATTCGCCTGGATGACCAGAAAGAGCGAGGAAACCATCACCCCCTGG
AACTTCGAGGAAGTGGTGGACAAGGGCGCCAGCGCCCAGAGCTTCATCGAGC
GGATGACCAACTTCGATAAGAACCTGCCCAACGAGAAGGTGCTGCCCAAGCAC
AGCCTGCTGTACGAGTACTTCACCGTGTACAACGAGCTGACCAAAGTGAAATA
CGTGACCGAGGGAATGAGAAAGCCCGCCTTCCTGAGCGGCGAGCAGAAAAAA
GCCATCGTGGACCTGCTGTTCAAGACCAACCGGAAAGTGACCGTGAAGCAGCT
GAAAGAGGACTACTTCAAGAAAATCGAGTGCTTCGACTCCGTGGAAATCTCCG
GCGTGGAAGATCGGTTCAACGCCTCCCTGGGCACATACCACGATCTGCTGAAA
ATTATCAAGGACAAGGACTTCCTGGACAATGAGGAAAACGAGGACATTCTGG
AAGATATCGTGCTGACCCTGACACTGTTTGAGGACAGAGAGATGATCGAGGAA
CGGCTGAAAACCTATGCCCACCTGTTCGACGACAAAGTGATGAAGCAGCTGAA
GCGGCGGAGATACACCGGCTGGGGCAGGCTGAGCCGGAAGCTGATCAACGGC
ATCCGGGACAAGCAGTCCGGCAAGACAATCCTGGATTTCCTGAAGTCCGACGG
CTTCGCCAACAGAAACTTCATGCAGCTGATCCACGACGACAGCCTGACCTTTA
AAGAGGACATCCAGAAAGCCCAGGTGTCCGGCCAGGGCGATAGCCTGCACGA
GCACATTGCCAATCTGGCCGGCAGCCCCGCCATTAAGAAGGGCATCCTGCAGA
CAGTGAAGGTGGTGGACGAGCTCGTGAAAGTGATGGGCCGGCACAAGCCCGA
GAACATCGTGATCGAAATGGCCAGAGAGAACCAGACCACCCAGAAGGGACAG
AAGAACAGCCGCGAGAGAATGAAGCGGATCGAAGAGGGCATCAAAGAGCTGG
GCAGCCAGATCCTGAAAGAACACCCCGTGGAAAACACCCAGCTGCAGAACGA
GAAGCTGTACCTGTACTACCTGCAGAATGGGCGGGATATGTACGTGGACCAGG
AACTGGACATCAACCGGCTGTCCGACTACGATGTGGACGCTATCGTGCCTCAG
AGCTTTCTGAAGGACGACTCCATCGATAACAAAGTGCTGACTCGGAGCGACAA
GAACCGGGGCAAGAGCGACAACGTGCCCTCCGAAGAGGTCGTGAAGAAGATG
AAGAACTACTGGCGCCAGCTGCTGAATGCCAAGCTGATTACCCAGAGGAAGTT
CGACAATCTGACCAAGGCCGAGAGAGGCGGCCTGAGCGAACTGGATAAGGCC
GGCTTCATCAAGAGACAGCTGGTGGAAACCCGGCAGATCACAAAGCACGTGG
CACAGATCCTGGACTCCCGGATGAACACTAAGTACGACGAGAACGACAAACT
GATCCGGGAAGTGAAAGTGATCACCCTGAAGTCCAAGCTGGTGTCCGATTTCC
GGAAGGATTTCCAGTTTTACAAAGTGCGCGAGATCAACAACTACCACCACGCC
CACGACGCCTACCTGAACGCCGTCGTGGGAACCGCCCTGATCAAAAAGTACCC
TAAGCTGGAAAGCGAGTTCGTGTACGGCGACTACAAGGTGTACGACGTGCGGA
AGATGATCGCCAAGAGCGAGCAGGAAATCGGCAAGGCTACCGCCAAGTACTT
CTTCTACAGCAACATCATGAACTTTTTCAAGACCGAGATTACCCTGGCCAACG
GCGAGATCCGGAAGCGGCCTCTGATCGAGACAAACGGCGAAACAGGCGAGAT
CGTGTGGGATAAGGGCCGGGACTTTGCCACCGTGCGGAAAGTGCTGTCTATGC
CCCAAGTGAATATCGTGAAAAAGACCGAGGTGCAGACAGGCGGCTTCAGCAA
AGAGTCTATCCTGCCCAAGAGGAACAGCGACAAGCTGATCGCCAGAAAGAAG
GACTGGGACCCTAAGAAGTACGGCGGCTTCGACAGCCCCACCGTGGCCTATTC
TGTGCTGGTGGTGGCCAAAGTGGAAAAGGGCAAGTCCAAGAAACTGAAGAGT
GTGAAAGAGCTGCTGGGGATCACCATCATGGAAAGAAGCAGCTTCGAGAAGA
ATCCCATCGACTTTCTGGAAGCCAAGGGCTACAAAGAAGTGAAAAAGGACCTG
ATCATCAAGCTGCCTAAGTACTCCCTGTTCGAGCTGGAAAACGGCCGGAAGAG
AATGCTGGCCTCTGCCGGCGAACTGCAGAAGGGAAACGAACTGGCCCTGCCCT
CCAAATATGTGAACTTCCTGTACCTGGCCAGCCACTATGAGAAGCTGAAGGGC
TCCCCCGAGGATAATGAGCAGAAACAGCTGTTTGTGGAACAGCACAAACACTA
CCTGGACGAGATCATCGAGCAGATCAGCGAGTTCTCCAAGAGAGTGATCCTGG
CCGACGCTAATCTGGACAAGGTGCTGAGCGCCTACAACAAGCACAGAGACAA
GCCTATCAGAGAGCAGGCCGAGAATATCATCCACCTGTTTACCCTGACCAATC
TGGGAGCCCCTGCCGCCTTCAAGTACTTTGACACCACCATCGACCGGAAGAGG
TACACCAGCACCAAAGAGGTGCTGGACGCCACCCTGATCCACCAGAGCATCAC
CGGCCTGTACGAGACACGGATCGACCTGTCTCAGCTGGGAGGCGAC
SEQ ID NO: 11-DNA Sequence of FOG1
ATGTCCAGGCGGAAACAGAGCAACCCCCGGCAGATCAAGCGTTCCCTCGGAG
ACATGGAGGCCAGAGAGGAGGTGCAGTTGGTGGGTGCCAGCCACATGGAGCA
AAAGGCCACGGCACCTGAAGCCCCGAGCCCT
SEQ ID NO: 12-DNA Sequence of mTagBFP
AGCGAGCTGATTAAGGAGAACATGCACATGAAGCTGTACATGGAGGGCACCG
TGGACAACCATCACTTCAAGTGCACATCCGAGGGCGAAGGCAAGCCCTACGAG
GGCACCCAGACCATGAGAATCAAGGTGGTCGAGGGCGGCCCTCTCCCCTTCGC
CTTCGACATCCTGGCTACTAGCTTCCTCTACGGCAGCAAGACCTTCATCAACCA
CACCCAGGGCATCCCCGACTTCTTCAAGCAGTCCTTCCCTGAGGGCTTCACATG
GGAGAGAGTCACCACATACGAAGACGGGGGCGTGCTGACCGCTACCCAGGAC
ACCAGCCTCCAGGACGGCTGCCTCATCTACAACGTCAAGATCAGAGGGGTGAA
CTTCACATCCAACGGCCCTGTGATGCAGAAGAAAACACTCGGCTGGGAGGCCT
TCACCGAGACGCTGTACCCCGCTGACGGCGGCCTGGAAGGCAGAAACGACAT
GGCCCTGAAGCTCGTGGGCGGGAGCCATCTGATCGCAAACATCAAGACCACAT
ATAGATCCAAGAAACCCGCTAAGAACCTCAAGATGCCTGGCGTCTACTATGTG
GACTACAGACTGGAAAGAATCAAGGAGGCCAACAACGAGACCTACGTCGAGC
AGCACGAGGTGGCAGTGGCCAGATACTGCGACCTCCCTAGCAAACTGGGGCAC
AAGCTTAAT
SEQ ID NO: 13-Amino Acid Sequence of the Triple Repressor BFP CRISPRi
System
NNSQGRVTFEDVTVNFTQGEWQRLNPEQRNLYRDVMLENYSNLVSVGQGETTKP
DVILRLEQGKEPWLEEEEVLGSGRAEKNGDIGGQIWKPKDVKESLGGSGGSVQVK
RVLEKSPGKLLVKMPFQASPGGKGEGGGATTSAQVMVIKRPGRKRKAEADPQAIP
KKRGRKPGSVVAAAAAEAKKKAVKESSIRSVQETVLPIKKRKTRETVSIEVKEVV
KPLLVSTLGEKSGKGLKTCKSPGRKSKESSPKGRSSSASSPPKKEHHHHHHHAESP
KAPMPLLPPPPPPEPQSSEDPISPPEPQDLSSSICKEEKMPRAGSLESDGCPKEPAKT
QPMVAAAATTTTTTTTTVAEKYKHRGEGERKDIVSSSMPRPNREEPVDSRTPVTE
RVSGSAGSAAGSGEFPKKKRKVRTDKKYSIGLAIGTNSVGWAVITDEYKVPSKKF
KVLGNTDRHSIKKNLIGALLFDSGETAEATRLKRTARRRYTRRKNRICYLQEIFSNE
MAKVDDSFFHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYNQLFEENPIN
ASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQE
EFYKFIKPILEKMDGTEELLVKLNREDLLRKQRTFDNGSIPHQIHLGELHAILRRQE
DFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTY
HDLLKIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLTFKED
IQKAQVSGQGDSLHEHIANLAGSPAIKKGILQTVKVVDELVKVMGRHKPENIVIEM
ARENQTTQKGQKNSRERMKRIEEGIKELGSQILKEHPVENTQLQNEKLYLYYLQN
GRDMYVDQELDINRLSDYDVDAIVPQSFLKDDSIDNKVLTRSDKNRGKSDNVPSE
EVVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQVNI
VKKTEVQTGGFSKESILPKRNSDKLIARKKDWDPKKYGGFDSPTVAYSVLVVAKV
EKGKSKKLKSVKELLGITIMERSSFEKNPIDFLEAKGYKEVKKDLIIKLPKYSLFELE
NGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLG
APAAFKYFDTTIDRKRYTSTKEVLDATLIHQSITGLYETRIDLSQLGGDAYPYDVPD
YASLGSGSPKKKRKVEGSSELIKENMHMKLYMEGTVDNHHFKCTSEGEGKPYEG
TQTMRIKVVEGGPLPFAFDILATSFLYGSKTFINHTQGIPDFFKQSFPEGFTWERVTT
YEDGGVLTATQDTSLQDGCLIYNVKIRGVNFTSNGPVMQKKTLGWEAFTETLYPA
DGGLEGRNDMALKLVGGSHLIANIKTTYRSKKPAKNLKMPGVYYVDYRLERIKE
ANNETYVEQHEVAVARYCDLPSKLGHKLNGSSKRPAATKKAGQAKKKKGGSGG
SGGSGGSMSRRKQSNPRQIKRSLGDMEAREEVQLVGASHMEQKATAPEAPSPGSG
ATNFSLLKQAGDVEENPGPMAKPLSQEESTLIERATATINSIPISEDYSVASAALSSD
GRIFTGVNVYHFTGGPCAELVVLGTAAAAAAGNLTCIVAIGNENRGILSPCGRCRQ
VLLDLHPGIKAIVKDSDGQPTAVGIRELLPSGYVWEG
SEQ ID NO: 14-Amino Acid Sequence of ZIM3 KRAB
NNSQGRVTFEDVTVNFTQGEWQRLNPEQRNLYRDVMLENYSNLVSVGQGETTKP
DVILRLEQGKEPWLEEEEVLGSGRAEKNGDIGGQIWKPKDVKESL
SEQ ID NO: 15-Amino Acid Sequence of MeCP2
VQVKRVLEKSPGKLLVKMPFQASPGGKGEGGGATTSAQVMVIKRPGRKRKAEAD
PQAIPKKRGRKPGSVVAAAAAEAKKKAVKESSIRSVQETVLPIKKRKTRETVSIEV
KEVVKPLLVSTLGEKSGKGLKTCKSPGRKSKESSPKGRSSSASSPPKKEHHHHHHH
AESPKAPMPLLPPPPPPEPQSSEDPISPPEPQDLSSSICKEEKMPRAGSLESDGCPKEP
AKTQPMVAAAATTTTTTTTTVAEKYKHRGEGERKDIVSSSMPRPNREEPVDSRTP
VTERVS
SEQ ID NO: 16-Amino Acid Sequence of dCas9 (Staphylococcus pyogenes)
DKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLI
EGDLNPDNSDVDKLFIQLVQTYNQLFEENPINASGVDAKAILSARLSKSRRLENLIA
QLPGEKKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEELLVKLNR
EDLLRKQRTFDNGSIPHQIHLGELHAILRRQEDFYPFLKDNREKIEKILTFRIPYYVG
PLARGNSRFAWMTRKSEETITPWNFEEVVDKGASAQSFIERMTNFDKNLPNEKVL
PKHSLLYEYFTVYNELTKVKYVTEGMRKPAFLSGEQKKAIVDLLFKTNRKVTVKQ
LKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQSG
KTILDFLKSDGFANRNFMQLIHDDSLTFKEDIQKAQVSGQGDSLHEHIANLAGSPAI
KKGILQTVKVVDELVKVMGRHKPENIVIEMARENQTTQKGQKNSRERMKRIEEGI
KELGSQILKEHPVENTQLQNEKLYLYYLQNGRDMYVDQELDINRLSDYDVDAIVP
QSFLKDDSIDNKVLTRSDKNRGKSDNVPSEEVVKKMKNYWRQLLNAKLITQRKF
DNLTKAERGGLSELDKAGFIKRQLVETRQITKHVAQILDSRMNTKYDENDKLIREV
KVITLKSKLVSDFRKDFQFYKVREINNYHHAHDAYLNAVVGTALIKKYPKLESEF
VYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVILADANLD
KVLSAYNKHRDKPIREQAENIIHLFTLTNLGAPAAFKYFDTTIDRKRYTSTKEVLD
ATLIHQSITGLYETRIDLSQLGGD
SEQ ID NO: 17-Amino Acid Sequence of FOG1
MSRRKQSNPRQIKRSLGDMEAREEVQLVGASHMEQKATAPEAPSP
SEQ ID NO: 18-Amino Acid Sequence of mTagBFP
SELIKENMHMKLYMEGTVDNHHFKCTSEGEGKPYEGTQTMRIKVVEGGPLPFAFD
ILATSFLYGSKTFINHTQGIPDFFKQSFPEGFTWERVTTYEDGGVLTATQDTSLQDG
CLIYNVKIRGVNFTSNGPVMQKKTLGWEAFTETLYPADGGLEGRNDMALKLVGG
SHLIANIKTTYRSKKPAKNLKMPGVYYVDYRLERIKEANNETYVEQHEVAVARYC
DLPSKLGHKLN
SEQ ID NO: 19-SV40 NLS
PKKKRKV
SEQ ID NO: 20-Nucleoplasmin NLS
KRPAATKKAGQAKKKK
SEQ ID NO: 21-Amino Acid Sequence of Linker #1
GGSGGS
SEQ ID NO: 22-Amino Acid Sequence of Linker #2
GSAGSAAGSGEF
SEQ ID NO: 23-Amino Acid Sequence of Linker #3
GGSGGSGGSGGS
SEQ ID NO: 24-EZH2 gRNA1
GCCCCGCTCGGCGATACCC
SEQ ID NO: 25-EZH2 gRNA2
GTCGCGTCCGACACCCGGT
SEQ ID NO: 26-ATF4 gRNA1
GACGAAGTCTATAAAGGGC
SEQ ID NO: 27-ATF4 gRNA2
CATGGCGTGAGTACCGGGG
SEQ ID NO: 28-HDAC1 gRNA1
ACCGACTGACGGTAGGGAC
SEQ ID NO: 29-HDAC1 gRNA2
GGACGGGAGGCGAGCAAGA
SEQ ID NO: 30-LMNA gRNA1
CGGACCTCGGGATCTGGGT
SEQ ID NO: 31-LMNA gRNA2
CCGGGCGCTGTCGGACCTC
SEQ ID NO: 32-RABIA gRNA
GCCGGCGAACCAGGAAATA
SEQ ID NO: 33-NTC gRNA
AACGTGCTGACGATGCGGGC
SEQ ID NO: 34-Sequencing Primer
GACCTGGGCAGATGTGGTT
SEQ ID NO: 35-Sequencing Primer
ACAGTCCCCGAGAAGTTGG
SEQ ID NO: 36-Sequencing Primer
AGAGAGTCTCGGTGGGTCTG
SEQ ID NO: 37-Sequencing Primer
AAAGAAGTGGTCAAGCCCCT
SEQ ID NO: 38-Sequencing Primer
CCAAGACCCAGCCTATGGT
SEQ ID NO: 39-Sequencing Primer
CAACACCGACCGGCACAG
SEQ ID NO: 40-Sequencing Primer
GGCTGATCTATCTGGCCCT
SEQ ID NO: 41-Sequencing Primer
GACACCTACGACGACGACCT
SEQ ID NO: 42-Sequencing Primer
GAACTGCTCGTGAAGCTGAA
SEQ ID NO: 43-Sequencing Primer
CCCAACGAGAAGGTGCTG
SEQ ID NO: 44-Sequencing Primer
GCTGACCCTGACACTGTTTG
SEQ ID NO: 45-Sequencing Primer
TCCTGCAGACAGTGAAGGTG
SEQ ID NO: 46-Sequencing Primer
ACAAAGTGCTGACTCGGAGC
SEQ ID NO: 47-Sequencing Primer
CAGTTTTACAAAGTGCGCGA
SEQ ID NO: 48-Sequencing Primer
GTGCTGTCTATGCCCCAAGT
SEQ ID NO: 49-Sequencing Primer
GTTCGAGCTGGAAAACGG
SEQ ID NO: 50-Sequencing Primer
CCCTGCCGCCTTCAAGTA
SEQ ID NO: 51-Sequencing Primer
GTTCCCTCGGAGACATGGAG
SEQ ID NO: 52-Sequencing Primer
GCAAACACAGTGCACACCAC
SEQ ID NO: 53-Sequencing Primer
GTGCTGACCGCTACCCAG
SEQ ID NO: 54-Amino acid sequence of Cas9 (Staphylococcus pyogenes)
MDKKYSIGLDIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHF
LIEGDLNPDNSDVDKLFIQLVQTYNQLFEENPINASGVDAKAILSARLSKSRRLENL
IAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLN
REDLLRKQRTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSP
AIKKGILQTVKVVDELVKVMGRHKPENIVIEMARENQTTQKGQKNSRERMKRIEE
GIKELGSQILKEHPVENTQLQNEKLYLYYLQNGRDMYVDQELDINRLSDYDVDHI
VPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIR
EVKVITLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKRYTSTKEVL
DATLIHQSITGLYETRIDLSQLGGD
SEQ ID NO: 55-Amino acid sequence of Cas9 (Staphylococcus aureus)
MKRNYILGLDIGITSVGYGIIDYETRDVIDAGVRLFKEANVENNEGRRSKRGARRL
KRRR
RHRIQRVKKLLFDYNLLTDHSELSGINPYEARVKGLSQKLSEEEFSAALLHLAKRR
GVHNVNEVEEDTGNELSTKEQISRNSKALEEKYVAELQLERLKKDGEVRGSINRF
KTSDYVKEAKQLLKVQKAYHQLDQSFIDTYIDLLETRRTYYEGPGEGSPFGWKDI
KEWYEMLMGHCTYFPEELRSVKYAYNADLYNALNDLNNLVITRDENEKLEYYEK
FQIIENVFKQKKKPTLKQIAKEILVNEEDIKGYRVTSTGKPEFTNLKVYHDIKDITAR
KEIIENAELLDQIAKILTIYQSSEDIQEELTNLNSELTQEEIEQISNLKGYTGTHNLSL
KAINLILDELWHTNDNQIAIFNRLKLVPKKVDLSQQKEIPTTLVDDFILSPVVKRSFI
QSIKVINAIIKKYGLPNDIIIELAREKNSKDAQKMINEMQKRNRQTNERIEEIIRTTG
KENAKYLIEKIKLHDMQEGKCLYSLEAIPLEDLLNNPFNYEVDHIIPRSVSFDNSFN
NKVLVKQEENSKKGNRTPFQYLSSSDSKISYETFKKHILNLAKGKGRISKTKKEYL
LEERDINRFSVQKDFINRNLVDTRYATRGLMNLLRSYFRVNNLDVKVKSINGGFTS
FLRRKWKFKKERNKGYKHHAEDALIIANADFIFKEWKKLDKAKKVMENQMFEEK
QAESMPEIETEQEYKEIFITPHQIKHIKDFKDYKYSHRVDKKPNRELINDTLYSTRK
DDKGNTLIVNNLNGLYDKDNDKLKKLINKSPEKLLMYHHDPQTYQKLKLIMEQY
GDEKNPLYKYYEETGNYLTKYSKKDNGPVIKKIKYYGNKLNAHLDITDDYPNSRN
KVVKLSLKPYRFDVYLDNGVYKFVTVKNLDVIKKENYYEVNSKCYEEAKKLKKI
SNQAEFIASFYNNDLIKINGELYRVIGVNNDLLNRIEVNMIDITYREYLENMNDKRP
PRIIKTIASKTQSIKKYSTDILGNLYEVKSKKHPQIIKKG
SEQ ID NO: 56-Amino acid sequence of Cas9 (Eubacterium ventriosum)
MGYTVGLDIGVASVGVAVLDENDNIVEAVSNIFDEADTSNNKVRRTLREGRRTKR
RQKTRIEDFKQLWETSGYIIPHKLHLNIIELRNKGLTELLSLDELYCVLLSMLKHRGI
SYLEDADDGEKGNAYKKGLAFNEKQLKEKMPCEIQLERMKKYGKYHGEFIIEIND
EKEYQSNVFTTKAYKKELEKIFETQRCNGNKINTKFIKKYMEIYERKREYYIGPGN
EKSRTDYGIYTTRTDEEGNFIDEKNIFGKLIGKCSVYPEEYRASSASYTAQEFNLLN
DLNNLKINNEKLTEFQKKEIVEIIKDASSVNMRKIIKKVIDEDIEQYSGARIDKKGKE
IYHTFEIYRKLKKELKTINVDIDSFTREELDKTMDILTLNTERESIVKAFDEQKFVYE
ENLIKKLIEFRKNNQRLFSGWHS
FSYKAMLQLIPVMYKEPKEQMQLLTEMNVFKSKKEKYVNYKYIPENEVVKEIYNP
VVVKSIRTTVKILNALIKKYGYPESVVIEMPRDKNSDDEKEKIDMNQKKNQEEYE
KILNKIYDEKGIEITNKDYKKQKKLVLKLKLWNEQEGLCLYSGKKIAIEDLLNHPE
FFEIDHIIPKSISL
DDSRSNKVLVYKTENSIKENDTPYHYLTRINGKWGFDEYKANVLELRRRGKIDDK
KVNNLLCMEDITKIDVVKGFINRNLNDTRYASRVVLNEMQSFFESRKYCNTKVKV
IRGSLTYQMRQDLHLKKNREESYSHHAVDAMLIAFSQKGYEAYRKIQKDCYDFET
GEILDKEKWNKYIDDDEFDDILYKERMNEIRKKIIEAEEKVKYNYKIDKKCNRGLC
NQTIYGTREKDGKIHKISSYNIYDDKECNSLKKMINSGKGSDLLMYNNDPKTYRD
MLKILETYSSEKNPFVAYNKETGDYFRKYSKNHNGPKVEKVKYYSGQINSCIDISH
KYGHAKNSKKVVLVSLNPYRTDVYYDNDTGKYYLVGVKYNHIKCVGNKYVIDS
ETYNELLRKEGVLNSDENLEDLNSKNITYKFSLYKNDIIQYEKGGEYYTERFLSRIK
EQKNLIETKPINKPNFQRKNKKGEWENTRNQIALAKTKYVGKLVTDVLGNCYIVN
MEKFSLVVDK
SEQ ID NO: 57-Amino acid sequence of Cas9 (Azospirillum)
MARPAFRAPRREHVNGWTPDPHRISKPFFILVSWHLLSRVVIDSSSGCFPGTSRDHT
DKF
AEWECAVQPYRLSFDLGTNSIGWGLLNLDRQGKPREIRALGSRIFSDGRDPQDKAS
LAVARRLARQMRRRRDRYLTRRTRLMGALVRFGLMPADPAARKRLEVAVDPYL
ARERATRERLEPFEIGRALFHLNQRRGYKPVRTATKPDEEAGKVKEAVERLEAAIA
AAGAPTLGAWFAWRKTRGETLRARLAGKGKEAAYPFYPARRMLEAEFDTLWAE
QARHHPDLLTAEAREILRHRIFHQRPLKPPPVGRCTLYPDDGRAPRALPSAQRLRLF
QELASLRVIHLDLSERPLTPAERDRIVAFVQGRPPKAGRKPGKVQKSVPFEKLRGL
LELPPGTGFSLESDKRPELLGDETGARIAPAFGPGWTALPLEEQDALVELLLTEAEP
ERAIAALTARWALDEATAAKLAGATLPDFHGRYGRRAVAELLPVLERETRGDPD
GRVRPIRLDEAVKLLRGGKDHSDFSREGALLDALPYYGAVLERHVAFGTGNPADP
EEKRVGRVANPTVHIALNQLRHLVNAILARHGRPEEIVIELARDLKRSAEDRRRED
KRQADNQKRNEERKRLILSLGERPTPRNLLKLRLWEEQGPVENRRCPYSGETISMR
MLLSEQVDIDHILPFSVSLDDSAANKVVCLREANRIKRNRSPWEAFGHDSERWAGI
LARAEALPKNKRWRFAPDALEKLEGEGGLRARHLNDTRHLSRLAVEYLRCVCPK
VRVSPGRLTALLRRRWGIDAILAEADGPPPEVPAETLDPSPAEKNRADHRHHALD
AVVIGCIDRSMVQRVQLAAASAEREAAAREDNIRRVLEGFKEEPWDGFRAELERR
ARTIVVSHRPEHGIGGALHKETAYGPVDPPEEGFNLVVRKPIDGLSKDEINSVRDPR
LRRALIDRLAIRRRDANDPATALAKAAEDLAAQPASRGIRRVRVLKKESNPIRVEH
GGNPSGPRSGGPFHKLLLAGEVHHVDVALRADGRRWVGHWVTLFEAHGGRGAD
GAAAPPRLGDGERFLMRLHKGDCLKLEHKGRVRVMQVVKLEPSSNSVVVVEPHQ
VKTDRSKHVKISCDQLRARGARRVTVDPLGRVRVHAPGARVGIGGDAGRTAMEP
AEDIS

1. A fusion protein, comprising:
a dCas9 protein; and
two or more repressor domains selected from the group consisting of:
(a) a Krüppel-associated box domain of ZIM3 gene (ZIM3 KRAB domain);
(b) a transcription repression domain of methyl-CpG binding protein 2 (MeCP2 domain); and
(c) a transcription repression domain of Friend of GATA1 (FOG1 domain).

2. The fusion protein according to claim 1, further comprising one or more nuclear localization sequences (NLSs).

3. The fusion protein according to claim 1, further comprising a fluorescent marker (FM).

4. The fusion protein according to claim 1, wherein the fusion protein is a ZIM3 KRAB-MeCP2-NLS-dCas9 fusion protein comprising from the N-terminus to the C-terminus: ZIM3 KRAB domain, MeCP2 domain, a NLS, and a dCas9 protein.

5. The fusion protein according to claim 1, wherein the fusion protein is a ZIM3 KRAB-NLS-dCas9-FOG1 fusion protein comprising from the N-terminus to the C-terminus: ZIM3 KRAB domain, a NLS, a dCas9 protein, and FOG1 domain, or wherein the fusion protein is a ZIM3 KRAB-NLS-dCas9-NLS-FOG1 fusion protein comprising from the N-terminus to the C-terminus: ZIM3 KRAB domain, a NLS, a dCas9 protein, a NLS, and FOG1 domain.

6. The fusion protein according to claim 1, wherein the fusion protein is a MeCP2-NLS-dCas9-FOG1 fusion protein comprising from the N-terminus to the C-terminus: MeCP2 domain, a NLS, a dCas9 protein, and FOG1 domain, or wherein the fusion protein is a MeCP2-NLS-dCas9-NLS-FOG1 fusion protein comprising from the N-terminus to the C-terminus: MeCP2 domain, a NLS, a dCas9 protein, a NLS, and FOG1 domain.

7. The fusion protein according to claim 1, wherein the fusion protein is a ZIM3 KRAB-MeCP2-NLS-dCas9-NLS-FOG1 fusion protein comprising from the N-terminus to the C-terminus: ZIM3 KRAB domain, MeCP2 domain, a NLS, a dCas9 protein, a NLS, and FOG1 domain.

8. The fusion protein according to claim 3, wherein the fusion protein is a ZIM3 KRAB-MeCP2-NLS-dCas9-NLS-FM-NLS-FOG1 fusion protein comprising from the N-terminus to the C-terminus: ZIM3 KRAB domain, MeCP2 domain, a NLS, a dCas9 protein, a NLS, a FM, a NLS, and FOG1 domain, optionally wherein the FM comprises mTagBFP.

9. The fusion protein according to claim 1, further comprising one or more linkers.

10. The fusion protein according to claim 1, wherein the dCas9 protein comprises at least one domain selected from the group consisting of: a Rec1 domain, a bridge helix domain, and a protospacer adjacent motif interacting domain.

11. The fusion protein according to claim 1, wherein the dCas9 protein comprises a D10A mutation in a RuvC1 domain and a H840A mutation in a HNH domain.

12. The fusion protein according to claim 1, wherein the dCas9 protein comprises an amino acid sequence having at least 95%, 96%, 97%, 98%, 99%, or 100% sequence identity to SEQ ID NO:16; and/or wherein ZIM3 KRAB domain comprises an amino acid sequence having at least 95%, 96%, 97%, 98%, 99%, or 100% sequence identity to SEQ ID NO:14; and/or wherein MeCP2 domain comprises an amino acid sequence having at least 95%, 96%, 97%, 98%, 99%, or 100% sequence identity to SEQ ID NO:15; and/or wherein FOG1 domain comprises an amino acid sequence having at least 95%, 96%, 97%, 98%, 99%, or 100% sequence identity to SEQ ID NO:17; and/or wherein mTagBFP comprises an amino acid sequence having at least 95%, 96%, 97%, 98%, 99%, or 100% sequence identity to SEQ ID NO:18; and/or wherein the NLS comprises at least one sequence having at least 95%, 96%, 97%, 98%, 99%, or 100% sequence identity to SEQ ID NO: 19 and/or SEQ ID NO:20.

13. The fusion protein according to claim 1, wherein the fusion protein comprises an amino acid sequence having at least 95%, 96%, 97%, 98%, 99%, or 100% sequence identity to SEQ ID NO:13, or an amino acid sequence having one, two, three, four, five or more amino acid substitutions, insertions, or deletions relative to SEQ ID NO:13.

14. A polynucleotide encoding the fusion protein according to claim 1.

15. The polynucleotide according to claim 14, wherein the polynucleotide comprises a sequence having at least 95%, 96%, 97%, 98%, 99%, or 100% sequence identity to SEQ ID NO: 7, or a sequence having one, two, three, four, five or more substitutions, insertions, or deletions relative to SEQ ID NO:7; and/or wherein the polynucleotide encoding the dCas9 protein comprises a sequence having at least 95%, 96%, 97%, 98%, 99%, or 100% sequence identity to SEQ ID NO:10; and/or wherein the polynucleotide encoding ZIM3 KRAB domain comprises a sequence having at least 95%, 96%, 97%, 98%, 99%, or 100% sequence identity to SEQ ID NO:8; and/or wherein the polynucleotide encoding MeCP2 domain comprises a sequence having at least 95%, 96%, 97%, 98%, 99%, or 100% sequence identity to SEQ ID NO:9; and/or wherein the polynucleotide encoding FOG1 domain comprises a sequence having at least 95%, 96%, 97%, 98%, 99%, or 100% sequence identity to SEQ ID NO:11; and/or wherein the polynucleotide encoding mTagBFP comprises a sequence having at least 95%, 96%, 97%, 98%, 99%, or 100% sequence identity to SEQ ID NO: 12.

16. A method of repressing expression of a gene in a cell, comprising contacting the cell with an effective amount of:
(a) the fusion protein according to claim 1 or the polynucleotide according to claim 14; and
(b) one or more gRNAs that bind the dCas9 protein according to claim 1.

17. The method according to claim 16, wherein the one or more gRNAs comprises a sequence having sufficient complementarity with a target polynucleotide sequence within the gene, and/or wherein the one or more gRNAs are capable of hybridizing with the target polynucleotide sequence.

18-21. (canceled)

22. A viral vector, comprising the polynucleotide according to claim 1, and optionally further comprising one or more gRNAs that bind the dCas9 protein according claim 1.

23. The viral vector according to claim 22, wherein the one or more gRNAs comprises a sequence having sufficient complementarity with a target polynucleotide sequence within the gene, and/or wherein the one or more gRNAs are capable of hybridizing with the target polynucleotide sequence.

24. The viral vector according to claim 22, wherein the viral vector comprises a lentiviral vector.


SEQUENCE LISTING (57 sequences)

SEQ ID NO: 1 - DNA, 1251 bp, synthetic construct
  source 1..1251: mol_type = other DNA
ctcgaggccaccatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctcggtgggtctggcggttctgtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctcaggctccgccggcagcgctgccggctcaggggagtttcctaagaaaaagcggaaagtgcgtacg

SEQ ID NO: 2 - DNA, 699 bp, synthetic construct
  source 1..699: mol_type = other DNA
ggatccagtaaacgacctgccgccactaaaaaagccggacaggctaagaagaagaaaggaggttcaggaggatctggggggagcggagggagcatgtccaggcggaaacagagcaacccccggcagatcaagcgttccctcggagacatggaggccagagaggaggtgcagttggtgggtgccagccacatggagcaaaaggccacggcacctgaagccccgagccctggctccggcgcaacaaacttctctctgctgaaacaagccggagatgtcgaagagaatcctggaccgatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagaattc

SEQ ID NO: 3 - DNA, 708 bp, synthetic construct
  source 1..708: mol_type = other DNA
ggatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatggatcc

SEQ ID NO: 4 - DNA, 12990 bp, synthetic construct
  source 1..12990: mol_type = other DNA
agtttgttttggcaccaaaatcaacgggactttccaaaatgtcgtaacaactccgccccattgacgcaaatgggcggtaggcgtgtacggtgggaggtctatataagcagcgcgttttgcctgtactggctctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagggttaattagctagcggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctcgaggccaccatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctccgtacggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaaggatccggcgcaacaaacttctctctgctgaaacaagccggagatgtcgaagagaatcctggaccgatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgagacgtttcatttccgtctctggtaccactttttaaaagaaaaggggggactggaagggctaattcactcccaacgaagtcaagatatccttgatctgtggatcgttaactaccacacacaaggctacttccctgattggcagaactacacaccagggccagggatcaaatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggagggttaacttaattaagacagccgcctagcatttcatcacatggcccgagagctgcatccggactgtactggctctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggccctctagagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatggg

SEQ ID NO: 5 - DNA, 13923 bp, synthetic construct
  source 1..13923: mol_type = other DNA
tcgaggccaccatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctcggtgggtctggcggttctgtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctcaggctccgccggcagcgctgccggctcaggggagtttcctaagaaaaagcggaaagtgcgtacggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaaggatccggcgcaacaaacttctctctgctgaaacaagccggagatgtcgaagagaatcctggaccgatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgagacgtttcatttccgtctctggtaccactttttaaaagaaaaggggggactggaagggctaattcactcccaacgaagtcaagatatccttgatctgtggatcgttaactaccacacacaaggctacttccctgattggcagaactacacaccagggccagggatcaaatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggagggttaacttaattaagacagccgcctagcatttcatcacatggcccgagagctgcatccggactgtactggctctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggccctctagagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactggctctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagggttaattagctagcggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggc

SEQ ID NO: 6 - DNA, 14151 bp, synthetic construct
  source 1..14151: mol_type = other DNA
gatccagtaaacgacctgccgccactaaaaaagccggacaggctaagaagaagaaaggaggttcaggaggatctggggggagcggagggagcatgtccaggcggaaacagagcaacccccggcagatcaagcgttccctcggagacatggaggccagagaggaggtgcagttggtgggtgccagccacatggagcaaaaggccacggcacctgaagccccgagccctggctccggcgcaacaaacttctctctgctgaaacaagccggagatgtcgaagagaatcctggaccgatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgagacgtttcatttccgtctctggtaccactttttaaaagaaaaggggggactggaagggctaattcactcccaacgaagtcaagatatccttgatctgtggatcgttaactaccacacacaaggctacttccctgattggcagaactacacaccagggccagggatcaaatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggagggttaacttaattaagacagccgcctagcatttcatcacatggcccgagagctgcatccggactgtactggctctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggccctctagagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactggctctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagggttaattagctagcggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctcgaggccaccatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctcggtgggtctggcggttctgtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctcaggctccgccggcagcgctgccggctcaggggagtttcctaagaaaaagcggaaagtgcgtacggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaag

SEQ ID NO: 7 - DNA, 14853 bp, synthetic construct
  source 1..14853: mol_type = other DNA
gatccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatggatccagtaaacgacctgccgccactaaaaaagccggacaggctaagaagaagaaaggaggttcaggaggatctggggggagcggagggagcatgtccaggcggaaacagagcaacccccggcagatcaagcgttccctcggagacatggaggccagagaggaggtgcagttggtgggtgccagccacatggagcaaaaggccacggcacctgaagccccgagccctggctccggcgcaacaaacttctctctgctgaaacaagccggagatgtcgaagagaatcctggaccgatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgagacgtttcatttccgtctctggtaccactttttaaaagaaaaggggggactggaagggctaattcactcccaacgaagtcaagatatccttgatctgtggatcgttaactaccacacacaaggctacttccctgattggcagaactacacaccagggccagggatcaaatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggagggttaacttaattaagacagccgcctagcatttcatcacatggcccgagagctgcatccggactgtactggctctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggccctctagagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactggctctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattagggttaattagctagcggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctcgaggccaccatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctcggtgggtctggcggttctgtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctcaggctccgccggcagcgctgccggctcaggggagtttcctaagaaaaagcggaaagtgcgtacggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaag

SEQ ID NO: 8 - DNA, 297 bp, synthetic construct
  source 1..297: mol_type = other DNA
aacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctc

SEQ ID NO: 9 - DNA, 858 bp, synthetic construct
  source 1..858: mol_type = other DNA
gtgcaggtgaaaagggtgctggaaaaatcccccggcaaactcctcgtgaagatgcccttccaggcttcccctggcggaaaaggtgaagggggtggcgcaaccacatctgcccaggtcatggtcatcaagcgacctggaaggaaaagaaaggccgaggctgaccctcaggccattccaaagaaacggggacgcaagccagggtccgtggtcgcagctgcagcagctgaggctaagaaaaaggcagtgaaggaaagctccatccgcagtgtgcaggagactgtcctgcccatcaagaagaggaagactagggagaccgtgtccatcgaggtcaaagaagtggtcaagcccctgctcgtgtccaccctgggcgaaaaatctggaaaggggctcaaaacatgcaagtcacctggacggaaaagcaaggagtctagtccaaaggggcgctcaagctccgcttctagtccccctaaaaaggaacaccatcaccatcaccatcacgccgagtctcctaaggctcctatgccactgctcccaccacctccaccacctgagccacagtcaagcgaagaccccatcagcccacccgagcctcaggatctgtcctctagtatttgcaaagaggaaaagatgcccagagcaggcagcctggagagtgatggctgtccaaaagaacccgccaagacccagcctatggtggcagccgctgcaactaccaccacaaccacaactaccacagtggccgaaaaatacaagcatcgcggcgagggcgaacgaaaggacattgtgtcaagctccatgcccagacctaaccgggaggaaccagtcgatagtaggacacccgtgactgagagagtctca

SEQ ID NO: 10 - DNA, 4101 bp, Staphylococcus pyogenes
  source 1..4101: mol_type = genomic DNA
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 11 - DNA, 135 bp, synthetic construct
  source 1..135: mol_type = other DNA
atgtccaggcggaaacagagcaacccccggcagatcaagcgttccctcggagacatggaggccagagaggaggtgcagttggtgggtgccagccacatggagcaaaaggccacggcacctgaagccccgagccct

SEQ ID NO: 12 - DNA, 696 bp, synthetic construct
  source 1..696: mol_type = other DNA
agcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaat

SEQ ID NO: 13 - protein, 2267 aa, synthetic construct
NNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLGGSGGSVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVSGSAGSAAGSGEFPKKKRKVRTDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNGSSKRPAATKKAGQAKKKKGGSGGSGGSGGSMSRRKQSNPRQIKRSLGDMEAREEVQLVGASHMEQKATAPEAPSPGSGATNFSLLKQAGDVEENPGPMAKPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 14 - protein, 99 aa, synthetic construct
NNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 15 - protein, 286 aa, synthetic construct
VQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVS

SEQ ID NO: 16 - protein, 1367 aa, Staphylococcus pyogenes
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 17 - protein, 45 aa, synthetic construct
MSRRKQSNPRQIKRSLGDMEAREEVQLVGASHMEQKATAPEAPSP

SEQ ID NO: 18 - protein, 232 aa, synthetic construct
SELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 19 - protein, 7 aa, synthetic construct
PKKKRKV

SEQ ID NO: 20 - protein, 16 aa, synthetic construct
KRPAATKKAGQAKKKK

SEQ ID NO: 21 - protein, 6 aa, synthetic construct
GGSGGS

SEQ ID NO: 22 - protein, 12 aa, synthetic construct
GSAGSAAGSGEF

SEQ ID NO: 23 - protein, 12 aa, synthetic construct
GGSGGSGGSGGS

SEQ ID NO: 24 - DNA, 19 bp, synthetic construct
  source 1..19: mol_type = other DNA
gccccgctcggcgataccc

SEQ ID NO: 25 - DNA, 19 bp, synthetic construct
  source 1..19: mol_type = other DNA
gtcgcgtccgacacccggt

SEQ ID NO: 26 - DNA, 19 bp, synthetic construct
  source 1..19: mol_type = other DNA
gacgaagtctataaagggc

SEQ ID NO: 27 - DNA, 19 bp, synthetic construct
  source 1..19: mol_type = other DNA
catggcgtgagtaccgggg

SEQ ID NO: 28 - DNA, 19 bp, synthetic construct
  source 1..19: mol_type = other DNA
accgactgacggtagggac

SEQ ID NO: 29 - DNA, 19 bp, synthetic construct
  source 1..19: mol_type = other DNA
ggacgggaggcgagcaaga

SEQ ID NO: 30 - DNA, 19 bp, synthetic construct
  source 1..19: mol_type = other DNA
cggacctcgggatctgggt

SEQ ID NO: 31 - DNA, 19 bp, synthetic construct
  source 1..19: mol_type = other DNA
cggacctcgggatctgggt

SEQ ID NO: 32 - DNA, 19 bp, synthetic construct
  source 1..19: mol_type = other DNA
gccggcgaaccaggaaata

SEQ ID NO: 33 - DNA, 20 bp, synthetic construct
  source 1..20: mol_type = other DNA
aacgtgctgacgatgcgggc

SEQ ID NO: 34 - DNA, 19 bp, synthetic construct
  source 1..19: mol_type = other DNA
gacctgggcagatgtggtt

SEQ ID NO: 35 - DNA, 19 bp, synthetic construct
  source 1..19: mol_type = other DNA
acagtccccgagaagttgg

SEQ ID NO: 36 - DNA, 20 bp, synthetic construct
  source 1..20: mol_type = other DNA
agagagtctcggtgggtctg

SEQ ID NO: 37 - DNA, 20 bp, synthetic construct
  source 1..20: mol_type = other DNA
aaagaagtggtcaagcccct

SEQ ID NO: 38 - DNA, 19 bp, synthetic construct
  source 1..19: mol_type = other DNA
ccaagacccagcctatggt

SEQ ID NO: 39 - DNA, 18 bp, synthetic construct
  source 1..18: mol_type = other DNA
caacaccgaccggcacag

SEQ ID NO: 40 - DNA, 19 bp, synthetic construct
  source 1..19: mol_type = other DNA
ggctgatctatctggccct

SEQ ID NO: 41 - DNA, 20 bp, synthetic construct
  source 1..20: mol_type = other DNA
gacacctacgacgacgacct

SEQ ID NO: 42 - DNA, 20 bp, synthetic construct
  source 1..20: mol_type = other DNA
gaactgctcgtgaagctgaa

SEQ ID NO: 43 - DNA, 18 bp, synthetic construct
  source 1..18: mol_type = other DNA
cccaacgagaaggtgctg

SEQ ID NO: 44 - DNA, 18 bp, synthetic construct
  source 1..18: mol_type = other DNA
cccaacgagaaggtgctg

SEQ ID NO: 45 - DNA, 20 bp, synthetic construct
  source 1..20: mol_type = other DNA
tcctgcagacagtgaaggtg

SEQ ID NO: 46 - DNA, 20 bp, synthetic construct
  source 1..20: mol_type = other DNA
acaaagtgctgactcggagc

SEQ ID NO: 47 - DNA, 20 bp, synthetic construct
  source 1..20: mol_type = other DNA
cagttttacaaagtgcgcga

SEQ ID NO: 48 - DNA, 20 bp, synthetic construct
  source 1..20: mol_type = other DNA
gtgctgtctatgccccaagt

SEQ ID NO: 49 - DNA, 18 bp, synthetic construct
  source 1..18: mol_type = other DNA
gttcgagctggaaaacgg

SEQ ID NO: 50 - DNA, 18 bp, synthetic construct
  source 1..18: mol_type = other DNA
ccctgccgccttcaagta

SEQ ID NO: 51 - DNA, 20 bp, synthetic construct
  source 1..20: mol_type = other DNA
gttccctcggagacatggag

SEQ ID NO: 52 - DNA, 20 bp, synthetic construct
  source 1..20: mol_type = other DNA
gcaaacacagtgcacaccac

SEQ ID NO: 53 - DNA, 18 bp, synthetic construct
  source 1..18: mol_type = other DNA
gtgctgaccgctacccag

SEQ ID NO: 54 - protein, 1368 aa, Staphylococcus pyogenes [sequence omitted]

SEQ ID NO: 55 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 56 - protein, 1107 aa, Eubacterium ventriosum
MGYTVGLDIGVASVGVAVLDENDNIVEAVSNIFDEADTSNNKVRRTLREGRRTKRRQKTRIEDFKQLWETSGYIIPHKLHLNIIELRNKGLTELLSLDELYCVLLSMLKHRGISYLEDADDGEKGNAYKKGLAFNEKQLKEKMPCEIQLERMKKYGKYHGEFIIEINDEKEYQSNVFTTKAYKKELEKIFETQRCNGNKINTKFIKKYMEIYERKREYYIGPGNEKSRTDYGIYTTRTDEEGNFIDEKNIFGKLIGKCSVYPEEYRASSASYTAQEFNLLNDLNNLKINNEKLTEFQKKEIVEIIKDASSVNMRKIIKKVIDEDIEQYSGARIDKKGKEIYHTFEIYRKLKKELKTINVDIDSFTREELDKTMDILTLNTERESIVKAFDEQKFVYEENLIKKLIEFRKNNQRLFSGWHSFSYKAMLQLIPVMYKEPKEQMQLLTEMNVFKSKKEKYVNYKYIPENEVVKEIYNPVVVKSIRTTVKILNALIKKYGYPESVVIEMPRDKNSDDEKEKIDMNQKKNQEEYEKILNKIYDEKGIEITNKDYKKQKKLVLKLKLWNEQEGLCLYSGKKIAIEDLLNHPEFFEIDHIIPKSISLDDSRSNKVLVYKTENSIKENDTPYHYLTRINGKWGFDEYKANVLELRRRGKIDDKKVNNLLCMEDITKIDVVKGFINRNLNDTRYASRVVLNEMQSFFESRKYCNTKVKVIRGSLTYQMRQDLHLKKNREESYSHHAVDAMLIAFSQKGYEAYRKIQKDCYDFETGEILDKEKWNKYIDDDEFDDILYKERMNEIRKKIIEAEEKVKYNYKIDKKCNRGLCNQTIYGTREKDGKIHKISSYNIYDDKECNSLKKMINSGKGSDLLMYNNDPKTYRDMLKILETYSSEKNPFVAYNKETGDYFRKYSKNHNGPKVEKVKYYSGQINSCIDISHKYGHAKNSKKVVLVSLNPYRTDVYYDNDTGKYYLVGVKYNHIKCVGNKYVIDSETYNELLRKEGVLNSDENLEDLNSKNITYKFSLYKNDIIQYEKGGEYYTERFLSRIKEQKNLIETKPINKPNFQRKNKKGEWENTRNQIALAKTKYVGKLVTDVLGNCYIVNMEKFSLVVDK

SEQ ID NO: 57 - protein, 1168 aa, Azospirillum sp.
MARPAFRAPRREHVNGWTPDPHRISKPFFILVSWHLLSRVVIDSSSGCFPGTSRDHTDKFAEWECAVQPYRLSFDLGTNSIGWGLLNLDRQGKPREIRALGSRIFSDGRDPQDKASLAVARRLARQMRRRRDRYLTRRTRLMGALVRFGLMPADPAARKRLEVAVDPYLARERATRERLEPFEIGRALFHLNQRRGYKPVRTATKPDEEAGKVKEAVERLEAAIAAAGAPTLGAWFAWRKTRGETLRARLAGKGKEAAYPFYPARRMLEAEFDTLWAEQARHHPDLLTAEAREILRHRIFHQRPLKPPPVGRCTLYPDDGRAPRALPSAQRLRLFQELASLRVIHLDLSERPLTPAERDRIVAFVQGRPPKAGRKPGKVQKSVPFEKLRGLLELPPGTGFSLESDKRPELLGDETGARIAPAFGPGWTALPLEEQDALVELLLTEAEPERAIAALTARWALDEATAAKLAGATLPDFHGRYGRRAVAELLPVLERETRGDPDGRVRPIRLDEAVKLLRGGKDHSDFSREGALLDALPYYGAVLERHVAFGTGNPADPEEKRVGRVANPTVHIALNQLRHLVNAILARHGRPEEIVIELARDLKRSAEDRRREDKRQADNQKRNEERKRLILSLGERPTPRNLLKLRLWEEQGPVENRRCPYSGETISMRMLLSEQVDIDHILPFSVSLDDSAANKVVCLREANRIKRNRSPWEAFGHDSERWAGILARAEALPKNKRWRFAPDALEKLEGEGGLRARHLNDTRHLSRLAVEYLRCVCPKVRVSPGRLTALLRRRWGIDAILAEADGPPPEVPAETLDPSPAEKNRADHRHHALDAVVIGCIDRSMVQRVQLAAASAEREAAAREDNIRRVLEGFKEEPWDGFRAELERRARTIVVSHRPEHGIGGALHKETAYGPVDPPEEGFNLVVRKPIDGLSKDEINSVRDPRLRRALIDRLAIRRRDANDPATALAKAAEDLAAQPASRGIRRVRVLKKESNPIRVEHGGNPSGPRSGGPFHKLLLAGEVHHVDVALRADGRRWVGHWVTLFEAHGGRGADGAAAPPRLGDGERFLMRLHKGDCLKLEHKGRVRVMQVVKLEPSSNSVVVVEPHQVKTDRSKHVKISCDQLRARGARRVTVDPLGRVRVHAPGARVGIGGDAGRTAMEPAEDIS